(19) United States
(12) Patent Application Publication
(10) Pub. No.: US 2024/0011908 A1
(43) Pub. Date: Jan. 11, 2024

(54) Rapid Pathology or Cytology, Particularly Without Wash

(71) Applicant: Essenlix Corporation, Monmouth Junction, NJ (US)

(72) Inventors: Stephen Y. CHOU, Princeton, NJ (US); Wei DING, Princeton, NJ (US); Yu Sun, Basking Ridge, NJ (US); Sheng-Jian Cai, Bridgewater, NJ (US); Wei DING, Princeton, NJ (US)

(73) Assignee: Essenlix Corporation, Monmouth Junction, NJ (US)

(21) Appl. No.: 18/034,557

(22) PCT Filed: Oct. 29, 2021
(86) PCT No.: PCT/US21/57455

§ 371 Date: Apr. 28, 2023

Related U.S. Application Data
(60) Provisional application No. 63/107,398, filed on Oct. 29, 2020.

(51) Int. Cl. G01N 21/64; G01N 1/30; G01N 1/28; G01N 33/569; C12Q 1/70

(52) U.S. Cl. CPC G01N 21/6458; G01N 1/30; G01N 1/2813; G01N 33/56983; C12Q 1/701; G01N 21/6428; G01N 2333/165; G01N 2021/6439

(57) ABSTRACT

Among other things, the disclosure of the present invention is related to make pathology and cytology faster, better, and lower cost, as well as to using fast cytology to quickly determine the concentration of the analyte outside a cell in a sample. The present invention is also related to rapid intra-cellular assays.

### CROSS-REFERENCE TO RELATED APPLICATIONS

This application claims the benefit of priority of U.S. Provisional Patent Application No. 63/107,398, filed on Oct. 29, 2020, the contents of which are relied upon and incorporated herein by reference in their entirety. The entire disclosure of any publication or patent document mentioned herein is entirely incorporated by reference.

### FIELD

The disclosure of the present invention is related to improve pathology and cytology.

### BACKGROUND

In biological and chemical assays (e.g. diagnostic testing), often it needs to stain and visualize analyte in biological samples quickly and simply for fast diagnostics. Furthermore, there are needs to reduce the steps, time and cost in pathology and cytology. Among other things, the disclosure of the present invention is related to make pathology and cytology faster, better and lower cost, as well as to using fast cytology to quickly determine the concentration of the analyte outside a cell in a sample The present invention also related to rapid intra-cellular assay.

### SUMMARY OF THE INVENTION

In some embodiments, the present invention provides a method of rapidly detecting an analyte inside a sample by sandwiching the sample between two plates, binding the analyte inside a cell with a probe, and imaging, wherein the distance spacing between the two plates and the probe concentration are optimized for detection of the analyte in the cell without washing, comprising: obtaining a sample that contains at least a cell, wherein the sample contains or is suspected of containing an analyte, wherein the concentration of the analyte inside the cell is higher than that outside the cell; obtaining a probe that binds the analyte, wherein the probe has a configured probe concentration; obtaining a sample holder comprising a first plate and a second plate that are arranged or can be arranged to face each other with a configured plate-spacing between them; sandwiching the sample and the probe between the two plates to form a sample-probe thin layer, wherein the sample-probe thin layer comprises a volume A and a volume B, wherein the thickness of the volume A and the volume B are regulated by the plate-spacing between two plates, and wherein volume A contains at least a fraction of the cell, and volume B does not contain the cell; imaging, after and without washing away the unbound probe and the two plates are arranged at the configured spacing, the sample-probe thin layer to produce one or more images, wherein the one or more images comprises the probe in volumes A and B; and comparing the signal of the probe inside the cell in the image of volume A to the signal of the probe in the image of volume B to determine whether the sample contains the analyte; wherein the configured probe concentration and the configured plate-spacing are configured to make the signal of the probe inside the cell in the image of volume A distinguishable from the signal of the probe in the image of volume B, and wherein the plate-spacing in imaged in step (e) is 250 um or less.

In some embodiments, the method further comprises: (i) obtaining, after step (b), a permeabilization agent, and (ii) sandwiching the permeabilization agent between the two plates and together with the sample and the probe.

In some embodiments, the invention provides a method of determining a disease and/or disorder of a subject, comprising: using the method of claim 1, wherein the sample is from the subject and the analyte is a biomarker of the disease and/or disorder; and determining a disease and/or disorder of a subject using the results.

In some embodiments, the imaging step is performed after the sandwiching step without separating the two plates.

In some embodiments, the disease and/or disorder is a viral infection, a bacterial infection, cancer or an inflammatory disease.

In some embodiments, the configured plate-spacing is regulated by spacers between the plates.

In some embodiments, the spacing between the two plates is configured to make the saturation time for the binding between the analyte and the probe is less than 300 seconds.

In some embodiments, each of volumes A and B has, during the imaging step, one surface in contact with the one of the two plates and another surface of in contact with other plate.

In some embodiments, the probe comprises a binding agent that binds specifically to the analyte.

In some embodiments, before the imaging step, the method comprises permeabilizing the cell.

In some embodiments, one or more of the plates comprises a dry permeabilizing agent and, in the sandwiching step, the permeabilizing agent dissolves in the sample and permeabilizes the cell.

In some embodiments, the probe is comprised by a staining solution, wherein the staining solution and the sample are sandwiched between the two plates in the imaging step.

In some embodiments, in the sample region being imaged in the imaging step, the spacing between the two plates is configured to make the saturation time for the binding between the analyte and the probe 60 seconds or less.

In some embodiments, the analyte is a protein, nucleic acid, or other macromolecule.

In some embodiments, the probe is a fluorescent or luminescent probe.

In some embodiments, the probe is a protein, nucleic acid, or aptamer.

In some embodiments, the cell is a free cell or a tissue section.

In some embodiments, the imaging of step is done using a mobile phone.

In some embodiments, the results of the comparing the signal step are transmitted to a remote location for analysis.

In some embodiments, the cell is a human cell.

In some embodiments, the cell is permeabilized either before or after the sample and the probe are sandwiched between the two plates.

In some embodiments, the configured plate-spacing is configured to make the cells in the sample forming a monolayer of cells between the two plates.

In some embodiments, one or both of the plates has a pillar that is used a reference for imaging the signal from the probe.

In some embodiments, the method is multiplexed and comprises binding of multiple probes to the cell in the sandwiching step and wherein the images of the imaging step comprise signals from the multiple probes.

In some embodiments, the sample is whole blood, without any liquid dilution.

In some embodiments, the sample is whole blood, without any liquid dilution

The method of any prior claim, wherein the probe is probe is coated on one or both plates before the sample is sandwiched between the two plates.

In some embodiments, the configured plate-spacing have a 0.5 um, 1 um, 2 um, 3 um, 5 um, 10 um, 15 um, 20 um, 30 um, 50 um, or a range between any two of the values.

In some embodiments, the configured plate-spacing is 0.5 um, 1 um, 2 um, 3 um, 5 um, 10 um, 15 um, or a range between any two of the values.

In some embodiments, the configured plate-spacing is 10 um.

In some embodiments, the imaging in step is performed within 300 seconds after the step (d) of sandwiching the sample.

In some embodiments, the first and second plates are movable relative to each other into different configurations, including an open configuration and a closed configuration, wherein the plate or the plates have spacers of uniform height on its surface, wherein in an open configuration, the two plates are completely or partially separated apart, the spacing between the plates is not regulated by the spacers, and wherein in a closed configuration, the thickness of the thin layer is regulated by the plates and the spacers.

In some embodiments, the spacers have a height of 0.5 um, 1 um, 2 um, 3 um, 5 um, 10 um, 15 um, 20 um, 30 um, 50 um, or a range between any two of the values.

In some embodiments, the method further comprises determining the concentration of analyte outside the cell in the sample by measuring the signal of the analyte inside the cells.

In some embodiments, thew method further comprises determining the concentration of analyte outside the cell in the sample by measuring the signal of the analyte inside the cells and by obtaining a relationship between the concentration of the cell-free analyte in a sample and the signal of the analyte inside a cell.

In some embodiments, the analyte is glycol-protein of virus.

In some embodiments, the analyte is RNA of virus.

In some embodiments, the analyte is RNA of COVID-19.

In some embodiments, the sample comprises bodily fluid selected from the group consisting of amniotic fluid, aqueous humour, vitreous humour, blood, breast milk, cerebrospinal fluid (CSF), cerumen (earwax), chyle, chime, endolymph, perilymph, feces, breath, gastric acid, gastric juice, lymph, mucus, pericardial fluid, peritoneal fluid, pleural fluid, pus, rheum, saliva, exhaled breath condensates, sebum, semen, sputum, sweat, synovial fluid, tears, vomit, urine, and any combination thereof.

One aspect of the present invention is to perform rapid pathology and cytology without washing. Particularly, the present invention is related to devices and methods that perform assaying (i.e. detection and/or staining) of an analyte in a sample in one step without washing, short incubation time (less than a few minutes, or 60 seconds) and immediately ready for reading signals.

Another aspect of the present invention is to improve sample staining and imaging by placing a comfortable plate with spacers of uniform height. The conformable plate can be rapidly prepared, is used to stain a tissue, and allows the tissue being imaged without washing.

Another aspect of the present invention is to stain and image a tissue using a conformable plate with spacers of uniform height by pressing the tissue to a thickness much less than the initial thickness.

Another aspect of the present invention is to improve tissue imaging using conformable flexible plate (i.e. film) of uniform micro-spacers.

Another aspect of the present invention is to perform rapid homogenous intracellular assay that detect the analyte inside the cell, and that can be much more sensitive than the assays that detect the same analyte outside the cell in a sample.

In some embodiments, the present invention provides a method of staining a tissue, comprising placing on a bottom plate a slice of tissue to be stained, providing a flexible top plate having, on its surface, spacers with a uniform height of 100 um or less, depositing a stain solution either on the tissue surface or on the flexible top plate, and sandwiching the sample and the stain solution between the top flexible plate and the bottom plate and pressing them together, wherein the flexibility of the top flexible plate and the spacing between the spacer are selected to make the top flexible plate conform to the surface of the tissue.

In some embodiments, the present invention provides a method of rapidly analyzing the pathology or cytology of a sample without washing the sample, comprising providing a first plate and a second plate; each, on its surface, having a sample contact area for contacting a sample that contains or is suspected of containing a target tissue or cell, providing a staining solution that stains the target tissue or cell, sandwiching the sample and the stain solution between the first and the second plates to form a thin layer of thickness of 200 um or less, and after sandwiching the sample and the stain solution between the first and the second plates to form a thin layer of thickness of 200 um or less, and without washing the sample to remove stain solution from the sample, imaging the thin layer to observe the stained target tissue or cell, wherein the thickness of the thin layer and the concentration of the stain solution in the thin layer are selected to make, during the imaging, the stained target tissue or cell in the thin layer is distinguishable from the rest of thin layer.

In some embodiments, the present invention provides a kit for analyzing the pathology of a sample without wash, comprising a first plate and a second plate, each, on its surface, having a sample contact area for contacting a sample that contains or is suspected of containing a target tissue or cell, and a staining solution that stains the target tissue or cell, wherein the first and second plates sandwich the sample and the stain solution into a thin layer of thickness that is regulated by the distance between the two sample contact areas, wherein the thin layer is imaged by an imager without washing the sample to remove stain solution from the sample, and wherein the distance between the two sample contact areas and the concentration of the stain solution in the thin layer are selected to make, during the imaging, the stained target tissue or cell in the thin layer is distinguishable from the rest of thin layer.

In some embodiments, the present invention provides an apparatus for analyzing the pathology of a sample without washing the sample, comprising a first plate and a second plate; each, on its surface, having a sample contact area for contacting a sample that contains or is suspected of containing a target tissue or cell, a staining solution that stains the target tissue or cell, and an imager, wherein the first and second plates sandwich the sample and the stain solution into a thin layer of a thickness that is regulated by the distance between the two sample contact areas, wherein the imager images the thin layer without washing the sample to remove stain solution from the sample, and wherein the distance between the two sample contact areas and the concentration of the stain solution in the thin layer are selected to make, during the imaging, the stained target tissue or cell in the thin layer is distinguishable from the rest of thin layer.

In some embodiments, the present invention provides a method for a rapid homogenous detection of an analyte inside a membrane of a cell in a sample, comprising the steps of (a) providing a first plate and a second plate, each, on its surface, having a sample contact area for contacting a sample comprising a cell that contains or is suspected of containing an analyte inside the cell, (b) providing a detection probe that (i) specifically binds the analyte and (ii) is capable of emitting a light at a wavelength, (c) providing a permeabilization reagent that makes a membrane of the cell permeable to the detection probe, wherein without the permeabilization reagent the detection probe cannot permeate into the cell, (d) sandwiching the sample, the detection probe, and the permeabilization reagent between the first and second plates to form a thin layer of a thickness of 200 microns (um) or less, and (e) after the step (d) and without washing the sample to remove unbound detection probe, imaging the thin layer to detect the cell that has the analyte bound to the detection probe, wherein the thickness of the thin layer and the concentration of the detection probe in the thin layer are selected to make, in the thin layer, the signal from the location having the detection probe bound to the analyte inside the cell distinguishable from signals from the locations that do not have the cell during the imaging of step (e). In some embodiments, the imaging of step (e) is performed 300 seconds or less after sandwiching of step (d). In one embodiment, the method further comprises a step of quantifying (i) the cell that has an analyte inside the cell and (ii) the cell that does have an analyte inside the cell. In another embodiment, the method further comprises a step of quantifying (i) the cell that has an analyte inside the cell and (ii) the cell that does have an analyte inside the cell, and a step of quantifying the percentage of the cell having an analyte inside the cell relative to the total number of the cell. In yet another embodiment, the light emitted by the detection probe is fluorescence, and the method further comprises (i) measuring the fluorescence intensity of the cell having the analyte bound to the detection probe, (ii) measuring the number of the cells having the analyte bound to the detection probe, and (iii) calculating a total fluorescence intensity by multiplying the total number of cells having the analyte bound to the detection probe in a unit area and the average of the fluorescence intensity of the cell having the analyte bound to the detection probe.

In some embodiments, the present invention provides a method of rapidly preparing and staining a tissue with an initial thickness thicker than the final thickness, comprising providing a first plate and a second plate that are movable relative to each other, wherein the second plate has spacers of uniform heights on its surface, placing, on the first plate, a tissue sample that has a thickness thicker than the spacer height, depositing a staining solution on the first plate or on the tissue, wherein the staining solution stains the tissue sample, and placing the first plate on top of the tissue sample and pressing to two plates together to make the tissue sample to form a thin layer, wherein the final thickness of the tissue sample layer is less than the initial thickness of the tissue sample and is regulated by the spacers and the two plates.

In some embodiments, the present invention provides a method of improving an imaging of a tissue surface, comprising providing a flexible plate, proving a spacers of uniform height, placing the spacer and the flexible plate on top of a tissue to be imaged, wherein the height of the spacer regulate the distance between the plate and the tissue, and wherein the spacer height is 50 um (micron) or less. In one embodiment, the uniform height is selected between 2 um to 20 um. In another embodiment, the uniform height is 10 um.

In some embodiments, the first and second plates are movable relative to each other into different configurations, including an open configuration and a closed configuration, wherein the plate or the plates have spacers of uniform height on its surface, wherein in an open configuration, the two plates are completely or partially separated apart, the spacing between the plates is not regulated by the spacers, and wherein in a closed configuration, the thickness of the thin layer is regulated by the plates, and the spacers, and has an average distance between the sample surface and the second plate surface is equal or less than 200 μm.

In some embodiments, the device, kit, and method further comprise the spacers that regulate the distance between the first plate and the second plate.

In some embodiments, the thickness of the thin layer is selected to make some of the cell having no overlap or significant overlap with other cells in the thin layer.

In some embodiments, the permeabilization reagent is coated on the surface of the plate or the plates.

In some embodiments, the permeabilization reagent is introduced to the sample before the sample and the permeabilization reagent are sandwiched between the two plates.

In some embodiment, the plate further has a dry stain reagent coated on its surface, and wherein the staining solution is a transfer liquid that transfers the dry stain agent into the sample.

In some embodiments, the stain solution is introduced to the sample before the sample and the permeabilization reagent are sandwiched between the two plates.

In one aspect, the present disclosure provides a method comprising obtaining a plate comprising one or more spacers fixed to a sample contact area on a surface thereof. In certain embodiments, the method comprises depositing a liquid onto at least one of (i) the sample contact area and (ii) a surface to be imaged. In certain embodiments, the method comprises contacting the one or more spacers with the surface to be imaged, thereby compressing at least a portion of the liquid into a layer of substantially uniform thickness confined by the plate and the surface to be imaged, wherein the uniform thickness of the layer is regulated by the height of the one or more spacers, and wherein the height of the one or more spacers is less than about 250 microns (um). In certain embodiments, the method comprises imaging the surface to be imaged.

In another aspect, the present disclosure provides a method comprises obtaining a first plate and a second plate, each comprising one or more spacers fixed to a sample contact area on a surface thereof. In certain embodiments, the method comprises depositing a staining liquid onto at least one of (i) the sample contact area of the first plate and (ii) a surface to be imaged. In certain embodiments, the method comprises contacting the one or more spacers of the first plate with the surface to be imaged. In certain embodiments, the method comprises, after a predetermined period of time, separating the first plate from the surface to be imaged. In certain embodiments, the method comprises depositing an imaging liquid onto at least one of (i) the sample contact area of the second plate and (ii) the surface to be imaged. In certain embodiments, the method comprises contacting the one or more spacers of the second plate with the surface to be imaged, thereby compressing at least a portion of the imaging liquid into a layer of substantially uniform thickness confined by the first plate and the surface to be imaged, wherein the uniform thickness of the layer is regulated by the height of the one or more spacers, and wherein the height of the one or more spacers is less than about 250 um. In certain embodiments, the method comprises imaging the surface to be imaged.

In one aspect, the present disclosure provides a device for analyzing a tissue sample, comprising a first plate; a second plate; a plurality of spacers; and a staining liquid, wherein the plates are movable relative to each other into different configurations; one or both plates are flexible; each of the plates has, on its respective inner surface, a sample contact area for contacting a staining liquid and/or a tissue sample contains or suspected of containing a target analyte; one or both of the plates comprise the spacers that are fixed with a respective plate; the spacers have a predetermined substantially uniform height and a predetermined inter-spacer distance; and at least one of the spacers is inside the sample contact area; wherein one of the configurations is an open configuration, in which: the two plates are partially or entirely separated apart, the spacing between the plates is not regulated by the spacers, and the staining liquid and the sample are deposited on one or both of the plates; and wherein another of the configurations is a closed configuration, which is configured after the deposition of the staining liquid and the sample in the open configuration, and in the closed configuration: at least part of the sample is between the two plates and a layer of at least part of staining liquid is between the at least part of the sample and the second plate, wherein the thickness of the at least part of staining liquid layer is regulated by the plates, the sample, and the spacers, and has an average distance between the sample surface and the second plate surface is equal to or less than 250 μm with a small variation.

In one aspect, the present disclosure provides a device for analyzing a tissue sample, comprising a first plate; a second plate; a plurality of spacers; a transfer solution; and a staining liquid, wherein the plates are movable relative to each other into different configurations; one or both plates are flexible; each of the plates has, on its respective inner surface, a sample contact area for contacting a transfer solution and/or a tissue sample contains or suspected of containing a target analyte; one or both of the plates comprise a stain agent that is coated on the respective sample contact area and configured to, upon contacting the transfer solution, be dissolved in the transfer solution and stain the tissue sample; one or both of the plates comprise the spacers that are fixed with a respective plate; the spacers have a predetermined substantially uniform height and a predetermined inter-spacer distance; and at least one of the spacers is inside the sample contact area; wherein one of the configurations is an open configuration, in which: the two plates are partially or entirely separated apart, the spacing between the plates is not regulated by the spacers, and the staining liquid and the sample are deposited on one or both of the plates; and wherein another of the configurations is a closed configuration, which is configured after the deposition of the staining liquid and the sample in the open configuration, and in the closed configuration: at least part of the sample is between the two plates and a layer of at least part of transfer solution is between the at least part of the sample and the second plate, wherein the thickness of the at least part of transfer solution layer is regulated by the plates, the sample, and the spacers, and has an average distance between the sample surface and the second plate surface is equal to or less than 250 μm with a small variation.

In one aspect, the present disclosure provides a method for analyzing a tissue sample, comprising the steps of providing a tissue sample contains or suspected of containing a target analyte; providing a staining liquid; providing a first plate, a second plate, and spacers, wherein the plates are movable relative to each other into different configurations, one or both plates are flexible; each of the plates has, on its respective inner surface, a sample contact area for contacting the staining liquid and/or the tissue sample; one or both of the plates comprise the spacers that are fixed with a respective plate; the spacers have a predetermined substantially uniform height and a predetermined inter-spacer distance; and at least one of the spacers is inside the sample contact area; depositing the staining liquid and the tissue sample on one or both of the plates when the plates are in an open configuration, wherein the open configuration is a configuration in which the two plates are partially or entirely separated apart, the spacing between the two plates is not regulated by the spacers, and the sample and the staining liquid are deposited on one or both of the plates; bringing the two plates together and pressing the plates into a closed configuration, wherein the pressing comprises conformable pressing, either in parallel or sequentially, an area of at least one of the plates to press the plates together to the closed configuration, wherein the conformable pressing generates a substantially uniform pressure on the plates over the at least part of the sample, and the pressing spreads the at least part of the sample laterally between the inner surfaces of the plates; and wherein another of the configurations is the closed configuration, which is configured after the deposition of the staining liquid and the sample in the open configuration, and in the closed configuration: at least part of the sample is between the two plates and a layer of at least part of staining liquid is between the at least part of the sample and the second plate, wherein the thickness of the at least part of staining liquid layer is regulated by the plates, the sample, and the spacers, and has an average distance between the sample surface and the second plate surface is equal to or less than 250 μm with a small variation; and analyzing the target analyte when the plates are in the closed configuration.

In one aspect, the present disclosure provides a method for analyzing a tissue sample, comprising the steps of obtaining a tissue sample contains or suspected of containing a target analyte and a transfer solution; obtaining a first plate, a second plate, and spacers, wherein the plates are movable relative to each other into different configurations; one or both plates are flexible; each of the plates has, on its respective inner surface, a sample contact area for contacting a staining liquid and/or a tissue sample suspected of containing a target analyte; one or both of the plates comprise stain agents that are coated on the respective sample contact area and configured to, upon contacting a transfer solution, be dissolved in the transfer solution and stain the tissue sample; one or both of the plates comprise the spacers that are fixed with a respective plate; the spacers have a predetermined substantially uniform height and a predetermined inter-spacer distance; and at least one of the spacers is inside the sample contact area; depositing the staining liquid and the tissue sample on one or both of the plates when the plates are in an open configuration, wherein the open configuration is a configuration in which the two plates are partially or entirely separated apart, the spacing between the two plates is not regulated by the spacers, and the sample and the staining liquid are deposited on one or both of the plates; bringing the two plates together and pressing the plates into a closed configuration, wherein the pressing comprises conformable pressing, either in parallel or sequentially, an area of at least one of the plates to press the plates together to the closed configuration, wherein the conformable pressing generates a substantially uniform pressure on the plates over the at least part of the sample, and the pressing spreads the at least part of the sample laterally between the inner surfaces of the plates; and wherein another of the configurations is the closed configuration, which is configured after the deposition of the staining liquid and the sample in the open configuration, and in the closed configuration: at least part of the sample is between the two plates and a layer of at least part of staining liquid is between the at least part of the sample and the second plate, wherein the thickness of the at least part of staining liquid layer is regulated by the plates, the sample, and the spacers, and has an average distance between the sample surface and the second plate surface is equal to or less than 250 μm with a small variation; and analyzing the target analyte when the plates are in the closed configuration.

In one aspect, the present disclosure provides a method for analyzing a tissue sample, comprising the steps of providing a tissue sample contains or suspected of containing a target analyte; providing a transfer solution and a stain agent; providing a first plate, a second plate, and spacers, wherein the plates are movable relative to each other into different configurations; one or both plates are flexible; each of the plates has, on its respective inner surface, a sample contact area for contacting the tissue sample; one or both of the plates comprise a stain agent that are coated on the respective sample contact area and configured to, upon contacting a transfer solution, be dissolved in the transfer solution to form a staining liquid and stain the tissue sample; one or both of the plates comprise the spacers that are fixed with a respective plate; the spacers have a predetermined substantially uniform height and a predetermined inter-spacer distance; and at least one of the spacers is inside the sample contact area; depositing the tissue sample on one or both of the plates when the plates are in an open configuration, wherein the open configuration is a configuration in which the two plates are partially or entirely separated apart, the spacing between the two plates is not regulated by the spacers, and the sample and the staining liquid are deposited on one or both of the plates; bringing the two plates together and pressing the plates into a closed configuration, wherein the pressing comprises conformable pressing, either in parallel or sequentially, an area of at least one of the plates to press the plates together to the closed configuration, wherein the conformable pressing generates a substantially uniform pressure on the plates over the at least part of the sample, and the pressing spreads the at least part of the sample laterally between the inner surfaces of the plates; and wherein another of the configurations is the closed configuration, which is configured after the deposition of the staining liquid and the sample in the open configuration, and in the closed configuration at least part of the sample is between the two plates and a layer of at least part of staining liquid is between the at least part of the sample and the second plate, wherein the thickness of the at least part of staining liquid is regulated by the plates, the sample, and the spacers, and has an average distance between the sample surface and the second plate surface is equal to or less than 250 μm with a small variation; and without washing, analyzing the target analyte when the plates are in the closed configuration.

In one aspect, the present disclosure provides, a system for analyzing a tissue sample, comprising the device of any embodiment of the present disclosure; and a detector configured to detecting signals of the target analyte in the layer of uniform thickness.

In one aspect, the present disclosure provides a smartphone system for tissue analysis assay, comprising the device of any embodiment of the present disclosure; and a mobile communication device that comprises one or a plurality of cameras for detecting and/or imaging the sample, electronics, signal processors, hardware and software for receiving and/or processing the detected signal and/or the image of the sample and for remote communication; and an adaptor that is configured to accommodate the device that is in the closed configuration and be engageable to the mobile communication device, wherein when engaged with the mobile communication device, the adaptor is configured to facilitate the detection and/or imaging of the target analyte in the sample at the closed configuration.

The device, system, smartphone system or method of any embodiment of the present disclosure, wherein one or both of the plates is configured such that the sample can be dried thereon at the open configuration, and wherein the sample comprises bodily fluid selected from the group consisting of amniotic fluid, aqueous humour, vitreous humour, blood, breast milk, cerebrospinal fluid (CSF), cerumen (earwax), chyle, chime, endolymph, perilymph, feces, breath, gastric acid, gastric juice, lymph, mucus, pericardial fluid, peritoneal fluid, pleural fluid, pus, rheum, saliva, exhaled breath condensates, sebum, semen, sputum, sweat, synovial fluid, tears, vomit, urine, and any combination thereof.

The device, system, smartphone system or method of embodiment of the present disclosure, wherein the blood is whole blood, fractionated blood, plasma or serum.

The device, system, smartphone system or method of embodiment of the present disclosure, wherein the mucus is nasal drainage or phlegm.

The device, system, smartphone system or method of any embodiment of the present disclosure, wherein the staining liquid has a viscosity in the range of 0.1 to 3.5 mPa S.

The device, system, smartphone system or method of any embodiment of the present disclosure, wherein the sample contact area of one or both of the plates is configured such that the sample is dried thereon on one or both plates at the open configuration, and wherein the sample comprises blood smear.

The device, system, smartphone system or method of any embodiment of the present disclosure, wherein the sample contact area of one or both of the plates is adhesive to the sample, and wherein the sample is a tissue section having a thickness in the range of 1-200 μm.

The device, system, smartphone system or method of any embodiment of the present disclosure, wherein the sample is paraffin-embedded.

The device, system, smartphone system or method of any embodiment of the present disclosure, wherein the sample is fixed.

The device, system, smartphone system or method of any embodiment of the present disclosure, wherein the staining liquid comprises a fixative capable of fixing the sample.

The device, system, smartphone system or method of any embodiment of the present disclosure, wherein the staining liquid comprises a blocking agent, wherein the blocking agent is configured to disable non-specific endogenous species in the sample to react with detection agents that are used to specifically label the target analyte.

The device, system, smartphone system or method of any embodiment of the present disclosure, wherein the staining liquid comprises a deparaffinizing agent capable of removing paraffin in the sample.

The device, system, smartphone system or method of any embodiment of the present disclosure, wherein the staining liquid comprises a permeabilizing agent capable of permeabilizing cells in the tissue sample that contain the target analyte.

The device, system, smartphone system or method of any embodiment of the present disclosure, wherein the staining liquid comprises an antigen retrieval agent capable of facilitating retrieval of antigen.

The device, system, smartphone system or method of any embodiment of the present disclosure, wherein the staining liquid comprises a detection agent that specifically label the target analyte in the sample.

The device, system, smartphone system or method of any embodiment of the present disclosure, wherein the sample contact area of one or both plates comprise a storage site that contains a blocking agent, wherein the blocking agent is configured to disable non-specific endogenous species in the sample to react with the detection agent that is used to specifically label the target analyte.

The device, system, smartphone system or method of any embodiment of the present disclosure, wherein the sample contact area of one or both plates comprise a storage site that contains a deparaffinizing agent capable of removing paraffin in the sample.

The device, system, smartphone system or method of any embodiment of the present disclosure, wherein the sample contact area of one or both plates comprise a storage site that contains a permeabilizing agent capable of permeabilizing cells in the tissue sample that contain the target analyte.

The device, system, smartphone system or method of any embodiment of the present disclosure, wherein the sample contact area of one or both plates comprise a storage site that contains an antigen retrieval agent capable of facilitating retrieval of antigen.

The device, system, smartphone system or method of any embodiment of the present disclosure, wherein the sample contact area of one or both plates comprise a storage site that contains a detection agent that specifically label the target analyte in the sample.

The device, system, smartphone system or method of any embodiment of the present disclosure, wherein the detection agent comprises a compound selected from the group consisting of: Acid fuchsin, Alcian blue 8 GX, Alizarin red S, Aniline blue WS, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, C.I. Cresyl violet, Crystal violet, Darrow red, Eosin B, Eosin Y, Erythrosin, Ethyl eosin, Ethyl green, Fast green F C F, Fluorescein Isothiocyanate, Giemsa Stain, Hematoxylin, Hematoxylin & Eosin, Indigo carmine, Janus green B, Jenner stain 1899, Light green SF, Malachite green, Martius yellow, Methyl orange, Methyl violet 2B, Methylene blue, Methylene blue, Methylene violet, (Bernthsen), Neutral red, Nigrosin, Nile blue A, Nuclear fast red, Oil Red, Orange G, Orange II, Orcein, Pararosaniline, Phloxin B, Protargol S, Pyronine B, Pyronine, Resazurin, Rose Bengal, Safranine O, Sudan black B, Sudan Ill, Sudan IV, Tetrachrome stain (MacNeal), Thionine, Toluidine blue, Weigert, Wright stain, and any combination thereof.

The device, system, smartphone system or method of any embodiment of the present disclosure, wherein one or both of the plates further comprise, on the respective sample contact area, a cell viability dye selected from the group consisting of: Propidium Iodide, 7-AAD, Trypan blue, Calcein Violet AM, Calcein AM, Fixable Viability Dyes, SYTO9 and other nucleic acid dyes, Resazurin and Formazan (MTT/XTT) and other mitochondrial dyes, and any combination thereof.

The device, system, smartphone system or method of any embodiment of the present disclosure, wherein the detection agent comprises an antibody configured to specifically bind to the target analyte in the sample.

The device, system, smartphone system or method of any prior claim, wherein the detection agent comprises an oligonucleotide probe configured to specifically bind to DNA and/or RNA in the sample.

The device, system, smartphone system or method of any embodiment of the present disclosure, wherein the detection agent is labeled with a reporter molecule, wherein the reporter molecule is configured to provide a detectable signal to be read and analyzed.

The device, system, smartphone system or method of embodiment of the present disclosure, wherein the signal is selected from the group consisting of luminescence selected from photoluminescence, electroluminescence, and electrochemiluminescence, light absorption, reflection, transmission, diffraction, scattering, or diffusion, surface Raman scattering, electrical impedance selected from resistance, capacitance, and inductance, magnetic relaxivity and a combination thereof.

The device, system, smartphone system or method of any embodiment of the present disclosure, wherein the sample contact area of one or both of the plates comprise a binding site that contains a capture agent, wherein the capture agent is configured to bind to the target analyte on the surface of cells in the sample and immobilize the cells.

The method of any embodiment of the present disclosure, wherein the depositing step further comprises the steps of depositing and drying the sample on one or both of the plates before depositing the remaining of the staining liquid on top of the dried sample, and wherein the sample comprises a blood smear that is dried on one or both plates.

The method of any embodiment of the present disclosure, wherein the depositing step further comprises the steps of depositing and attaching the sample to one or both of the plates before depositing the staining liquid on top of the sample, wherein the sample contact area of one or both of the plates is adhesive to the sample, and wherein the sample is a tissue section having a thickness in the range of 1-200 □m.

The method of any embodiment of the present disclosure, before the analyzing step (e), further comprising: incubating the sample at the closed configuration for a period of time that is longer than the time it takes for the detection agent to diffuse across the layer of uniform thickness and the sample.

The method of any embodiment of the present disclosure, before the analyzing step (e), further comprising the step of incubating the sample at the closed configuration at a predetermined temperature in the range of 30-75° C.

The method of any embodiment of the present disclosure, wherein the staining liquid comprises the transfer solution.

The smartphone system of any embodiment of the present disclosure, wherein the mobile communication device is configured to communicate test results to a medical professional, a medical facility or an insurance company.

The smartphone system of any embodiment of the present disclosure, wherein the mobile communication device is further configured to communicate information on the subject with the medical professional, medical facility or insurance company.

The smartphone system of any embodiment of the present disclosure, wherein the mobile communication device is configured to receive a prescription, diagnosis or a recommendation from a medical professional.

The smartphone system of any embodiment of the present disclosure, wherein the mobile communication device communicates with the remote location via a WIFI or cellular network.

The smartphone system of any embodiment of the present disclosure, wherein the mobile communication device is a mobile phone.

The device, system, smartphone system or method of any embodiment of the present disclosure, wherein the pressing is performed by a human hand.

The device, system, smartphone system or method of any embodiment of the present disclosure, wherein at least a portion of the inner surface of one plate or both plates is hydrophilic.

The device, system, smartphone system or method of any embodiment of the present disclosure, wherein the inter spacer distance is periodic.

The device, system, smartphone system or method of any embodiment of the present disclosure, wherein the sample is a deposition directly from a subject to the plate without using any transferring devices.

The device, system, smartphone system or method of any embodiment of the present disclosure, wherein after the sample deformation at a closed configuration, the sample maintains the same final sample thickness, when some or all of the compressing forces are removed.

The device, system, smartphone system or method of any embodiment of the present disclosure, wherein the spacers have pillar shape and nearly uniform cross-section.

The device, system, smartphone system or method of any embodiment of the present disclosure, wherein the inter spacer distance (SD) is equal or less than about 120 μm (micrometer).

The device, system, smartphone system or method of any embodiment of the present disclosure, wherein the inter spacer distance (SD) is equal or less than about 100 μm (micrometer).

The device, system, smartphone system or method of any embodiment of the present disclosure, wherein the fourth power of the inter-spacer-distance (ISD) divided by the thickness (h) and the Young's modulus (E) of the flexible plate (ISD⁴/(hE)) is 5×10⁶ μm³/GPa or less.

The device, system, smartphone system or method of any embodiment of the present disclosure, wherein the fourth power of the inter-spacer-distance (ISD) divided by the thickness (h) and the Young's modulus (E) of the flexible plate (ISD⁴/(hE)) is 5×10⁵ μm³/GPa or less.

The device, system, smartphone system or method of any embodiment of the present disclosure, wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one).

The device, system, smartphone system or method of any embodiment of the present disclosure, wherein the spacers have pillar shape, a substantially flat top surface, a predetermined substantially uniform height, and a predetermined constant inter-spacer distance that is at least about 2 times larger than the size of the analyte, wherein the Young's modulus of the spacers times the filling factor of the spacers is equal or larger than 2 MPa, wherein the filling factor is the ratio of the spacer contact area to the total plate area, and wherein, for each spacer, the ratio of the lateral dimension of the spacer to its height is at least 1 (one), wherein the fourth power of the inter-spacer-distance (ISD) divided by the thickness (h) and the Young's modulus (E) of the flexible plate (ISD⁴/(hE)) is 5×10⁶ μm³/GPa or less.

The device, system, smartphone system or method of any embodiment of the present disclosure, wherein the ratio of the inter-spacing distance of the spacers to the average width of the spacer is 2 or larger, and the filling factor of the spacers multiplied by the Young's modulus of the spacers is 2 MPa or larger.

The device, kit, system, smartphone system, and method of any embodiment of the present disclosure, wherein the target analytes is a protein, peptide, nucleic acid, synthetic compound, or an inorganic compound.

The device, system, smartphone system, and method of any embodiment of the present disclosure, wherein the sample is a biological sample selected from amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, and urine.

The device, system, smartphone system, and method of any embodiment of the present disclosure, wherein the spacers have a shape of pillars and a ratio of the width to the height of the pillar is equal or larger than one.

The device, system, smartphone system or method of any embodiment of the present disclosure, wherein the sample that is deposited on one or both of the plates has an unknown volume.

The device, system, smartphone system, and method of any embodiment of the present disclosure, wherein the spacers have a shape of pillar, and the pillar has substantially uniform cross-section.

The device, system, smartphone system, and method of any embodiment of the present disclosure, wherein the samples are for the detection, purification and quantification of chemical compounds or biomolecules that correlates with the stage of certain diseases.

The device, system, smartphone system, and method of any embodiment of the present disclosure, wherein the sample is related to infectious and parasitic disease, injuries, cardiovascular disease, cancer, mental disorders, neuropsychiatric disorders, pulmonary diseases, renal diseases, and other and organic diseases.

The device, system, smartphone system, and method of any embodiment of the present disclosure, wherein the samples are related to the detection, purification and quantification of microorganism.

The device, system, smartphone system, and method of any embodiment of the present disclosure, wherein the samples is related to a virus, fungus and bacterium from environment, e.g., water, soil, or biological samples.

The device, system, smartphone system, and method of any embodiment of the present disclosure, wherein the samples is related to the detection, quantification of chemical compounds or biological samples that pose hazard to food safety or national security, e.g. toxic waste, anthrax.

The device, system, smartphone system, and method of any embodiment of the present disclosure, wherein the samples are related to quantification of vital parameters in medical or physiological monitor.

The device, system, smartphone system, and method of any embodiment of the present disclosure, wherein the samples are related to glucose, blood, oxygen level, total blood count.

The device, system, smartphone system, and method of any embodiment of the present disclosure, wherein the samples are related to the detection and quantification of specific DNA or RNA from bio-samples.

The device, system, smartphone system, and method of any embodiment of the present disclosure, wherein the samples are related to the sequencing and comparing of genetic sequences in DNA in the chromosomes and mitochondria for genome analysis.

The device, system, smartphone system, and method of any embodiment of the present disclosure, wherein the samples are related to detect reaction products, e.g., during synthesis or purification of pharmaceuticals.

The device, system, smartphone system, and method of any embodiment of the present disclosure, wherein the samples are cells, tissues, bodily fluids, and stool.

The device, system, smartphone system, and method of any embodiment of the present disclosure, wherein the sample is the sample in the detection of proteins, peptides, nucleic acids, synthetic compounds, inorganic compounds.

The device, system, smartphone system, and method of any embodiment of the present disclosure, wherein the sample is the sample in the fields of human, veterinary, agriculture, foods, environments, and drug testing.

The device, system, smartphone system or method of any embodiment of the present disclosure, wherein the sample is a biological sample is selected from blood, serum, plasma, a nasal swab, a nasopharyngeal wash, saliva, urine, gastric fluid, spinal fluid, tears, stool, mucus, sweat, earwax, oil, a glandular secretion, cerebral spinal fluid, tissue, semen, vaginal fluid, interstitial fluids derived from tumorous tissue, ocular fluids, spinal fluid, a throat swab, breath, hair, finger nails, skin, biopsy, placental fluid, amniotic fluid, cord blood, lymphatic fluids, cavity fluids, sputum, pus, microbiota, meconium, breast milk, exhaled condensate nasopharyngeal wash, nasal swab, throat swab, stool samples, hair, finger nail, ear wax, breath, connective tissue, muscle tissue, nervous tissue, epithelial tissue, cartilage, cancerous sample, and bone.

### BRIEF DESCRIPTION OF THE DRAWINGS

A skilled artisan will understand that the drawings, described below, are for illustration purposes only. The drawings are not intended to limit the scope of the present teachings in any way. The drawings are not entirely in scale. In the figures that present experimental data points, the lines that connect the data points are for guiding a viewing of the data only and have no other means.

FIG. 1 illustrates an embodiment of the present disclosure, in which a conformable plate comprising spacers conforms to a shape of the surface of a sample.

FIG. 2. Schematic figure of experimental procedure using iMOST Q-CARD microvolume embodiment. 1, place oral Q-tip brush biopsy or chicken stomach tissue biopsy (5 mm×1 mm) on sub-card or glass slide; 2, drop 10 ul (volume can be varied based on the size of tissue and pillar height) staining solution on the pillar side of X-plate; 3, place X-plate on top of glass slide with staining solution facing down towards tissues; 4, press X-plate onto tissue to allow staining solution mounting the whole piece of tissue, and to spread the tissue into micrometer thin layer (which thickness determined by pillar height); 5, leave MOST Q-Card embodiment at room temperature for 1 min and observe under microscopy. (Note: The Term “Q-CARD” and “QMAX Card” are interchangeable.)

FIG. 3 Bright field microscopy image of 1 step PAP staining of oral mucosa cells (OMCs) using Q-Card. In the experiment, the Q-Card has a spacer height of 10 um with a pillar array with pillar size of 30 um by 40 um size and period of 110 um by 120 um. The OMC cells are stained to red color in the image.

FIG. 4. Chicken stomach mucosa cytology staining by comformal press thinning with hematoxylin. Initially, a thin layer single chicken stomach (epithelial cells) tissue was ˜1 mm thick. By press thinning, the tissue was significantly thinned and some location has nearly the same height as the pillar height (10 um). However, the pressed cells were not damaged. The chicken stomach epithelial cells were hematoxylin positively stained (100×, left panel) with blue/purple color (left panel). With higher magnification, the functional unit, mucosa crypt, was able to be visualized as in the green circle (right panel).

FIG. 5 illustrates a comparison between fluorescence images obtained with (i) one-step staining using an X-plate with the spacers contacting the tissue, and (ii) using flat palte. The image using X-plate have better image contrast and sharper image than that of the flat plate.

FIG. 6. Illustration of intra-cellular assay. A sample with a cell sandwiched between two plates, wherein the cell contains analytes (i.e. biomarkers) inside of the cell, and some of analytes are outside the cell. In some embodiments, the analyte concentration inside the cell has a colleration with that outside the cell.

FIG. 7 illustrates a flow chart of one exemplary embodiment of the method with the steps of (a) collecting urine samples containing cells using a container; (b) transferring a drop of urine sample onto one or both of the plates the device (i.e. Q-Card) in an open configuration; (c) closing the device and imaging, without a wash, the cell sandwiched by two plates (plate 1 and plate 2) of the Q-Card. In some embodiments, the gap (i.e. the spacing between the plates) and the concentration of reagent between the two plates are configured, so that at the closed configuration (i) cells in the sample forming a monolayer (i.e. no significant lateral overlap), and (ii) a stain cell is visible without washing. In some embodiment, a staining reagents are coated on the plates (either dry reagent or a liquid reagent) and dissolved into the urine when in a closed configuration.

FIG. 8 illustrates a flow chart of one exemplary embodiment of the method with the steps of (1) collecting urine samples with cells inside a container; (2) staining the cells in the urine sample by mixing staining reagents with urine sample, (3) transferring a drop of urine sample onto the device in open configuration; (4) closing the device and imaging the cell sandwiched by two plates (plate 1 and plate 2) with a gap naming “SNT”, which ensures (i) cells forming a monolayer and (ii) sub noise thick for imaging.

FIG. 9 illustrates a flow chart of one exemplary embodiment of the method with the steps of (1) collecting urine samples with cells inside a container; (2) staining the cells in the urine sample by mixing staining reagents with urine sample, (3) transferring a drop of urine sample onto the device in open configuration; (4) closing the device and imaging the cell sandwiched by two plates (plate 1 and plate 2).

FIG. 10 illustrates example bright field microscopy images of Haematoxylin stained urine cells inside the device, wherein the device has a gap of 10 um, a pillar array of 30 by 40 um pillar size and 80 um inter spacing distance. The labeled cell inside the images are mainly stained epithelial cells.

FIG. 11 illustrates example fluorescence images taken by smartphone of Acridine orange stained urine cells inside the device, wherein the device has a gap of 30 um, a pillar array of 30 by 40 um pillar size and 80 um inter spacing distance. The labeled cell inside the images are mainly stained epithelial cells and white blood cells.

FIG. 12 illustrates of regulating the plate-spacing by putting spacers between the two plates of a sample holder, and the density of the spacer is controlled to have less effects on the cells in the sample that is sandwiches between the plate.

### DETAILED DESCRIPTION OF EXEMPLARY EMBODIMENTS

The following detailed description illustrates some embodiments of the invention by way of example and not by way of limitation. The section headings and any subtitles used herein are for organizational purposes only and are not to be construed as limiting the subject matter described in any way. The contents under a section heading and/or subtitle are not limited to the section heading and/or subtitle, but apply to the entire description of the present disclosure.

The citation of any publication is for its disclosure prior to the filing date and should not be construed as an admission that the present claims are not entitled to antedate such publication by virtue of prior invention. Further, the dates of publication provided can be different from the actual publication dates which can need to be independently confirmed.

The present invention work for both tissues and other biological samples with cells, and in some descriptions they mention tissue samples without mentioning samples with cells; however, it is understood that the device and methods work for the samples with cells (e.g. mouth or nose swaps).

### Definitions

The term “present disclosure” and “present invention” are interchangeable.

The term “FAST” (all in capital letters) means the “fast staining” of the present invention, which includes, but not limited to, all fast staining devices and/or methods described by the present invention. The terms “X-Path” and “FAST” are interchangeable.

The terms “perform, using a Q-Card, an assay (including staining in pathology) without using any wash” and “perform, using a Q-Card, an assay (including staining in pathology) wash-free” are interchangeable.

The terms “in a closed configuration” and “at a closed configuration” for the plates of the Q-Card are interchangeable.

The terms “analyte” and “biomarker” are interchangeable

The term “permeabilizing” a cell refers make the cell to allow large molecules like antibodies and/or nucleic acid to get inside the cell.

The term “multiplexing” is the use of multiple probes. The term “staining solution” and “staining liquid” are interchangeable.

The term “inner surface” of the first and second plates is the surfaces that are facing each other in a closed configuration.

The terms “specific binding” and “selective binding” refer to the ability of a capture agent/detection agent to preferentially bind to a particular target molecule that is present in a heterogeneous mixture of different target molecule. A specific or selective binding interaction will discriminate between desirable (e.g., active) and undesirable (e.g., inactive) target molecules in a sample, typically more than about 10 to 100-fold or more (e.g., more than about 1000- or 10,000-fold).

The term “probe”, “capture agent” and “detection agent” are interchangeable.

A “biomarker,” as used herein, is any molecule or compound that is found in a sample of interest and that is known to be diagnostic of or associated with the presence of or a predisposition to a disease or condition of interest in the subject from which the sample is derived. Biomarkers include, but are not limited to, polypeptides or a complex thereof (e.g., antigen, antibody), nucleic acids (e.g., DNA, miRNA, mRNA), drug metabolites, lipids, carbohydrates, hormones, vitamins, etc., that are known to be associated with a disease or condition of interest.

The terms “polynucleotide”, “nucleotide”, “nucleotide sequence”, “nucleic acid”, “nucleic acid molecule”, “nucleic acid sequence” and “oligonucleotide” are used interchangeably, and can also include plurals of each respectively depending on the context in which the terms are utilized. They refer to a polymeric form of nucleotides of any length, either deoxyribonucleotides (DNA) or ribonucleotides (RNA), or analogs thereof.

Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA (tRNA), ribosomal RNA, ribozymes, small interfering RNA, (siRNA), microRNA (miRNA), small nuclear RNA (snRNA), cDNA, recombinant polynucleotides, branched polynucleotides, plasmids, vectors, isolated DNA (A, B and Z structures) of any sequence, PNA, locked nucleic acid (LNA), TNA (treose nucleic acid), isolated RNA of any sequence, nucleic acid probes, and primers. LNA, often referred to as inaccessible RNA, is a modified RNA nucleotide. The ribose moiety of an LNA nucleotide is modified with an extra bridge connecting the 2′ and 4′ carbons. The bridge “locks” the ribose in the 3′-endo structural conformation, which is often found in the A-form of DNA or RNA, which can significantly improve thermal stability.

The term “capture agent” as used herein, refers to a binding member, e.g. nucleic acid molecule, polypeptide molecule, or any other molecule or compound, that can specifically bind to its binding partner, e.g., a second nucleic acid molecule containing nucleotide sequences complementary to a first nucleic acid molecule, an antibody that specifically recognizes an antigen, an antigen specifically recognized by an antibody, a nucleic acid aptamer that can specifically bind to a target molecule, etc. A capture agent may concentrate the target molecule from a heterogeneous mixture of different molecules by specifically binding to the target molecule. Binding may be non-covalent or covalent. The affinity between a binding member and its binding partner to which it specifically binds when they are specifically bound to each other in a binding complex is characterized by a K_{D} (dissociation constant) of 10⁻⁵ M or less, 10⁻⁶ M or less, such as 10⁻⁷ M or less, including 10⁻⁸ M or less, e.g., 10⁻⁹ M or less, 10⁻¹⁰ M or less, 10⁻¹¹ M or less, 10⁻¹² M or less, 10⁻¹³ M or less, 10⁻¹⁴ M or less, 10⁻¹⁵ M or less, including 10⁻¹⁶ M or less. “Affinity” refers to the strength of binding, increased binding affinity being correlated with a lower K_{D}.

The term “antibody,” as used herein, is meant a protein consisting of one or more polypeptides substantially encoded by all or part of the recognized immunoglobulin genes. The recognized immunoglobulin genes, for example in humans, include the kappa (κ), lambda (λ), and heavy chain genetic loci, which together comprise the myriad variable region genes, and the constant region genes mu (μ), delta (δ), gamma (γ), sigma (σ), and alpha (α) which encode the IgM, IgD, IgG, IgE, and IgA antibody “isotypes” or “classes” respectively. Antibody herein is meant to include full length antibodies and antibody fragments, and may refer to a natural antibody from any organism, an engineered antibody, or an antibody generated recombinantly for experimental, therapeutic, or other purposes. The term “antibody” includes full length antibodies, and antibody fragments, as are known in the art, such as Fab, Fab′, F(ab′)2, Fv, scFv, or other antigen-binding subsequences of antibodies, either produced by the modification of whole antibodies or those synthesized de novo using recombinant DNA technologies.

The term “Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these.

As is known to one skilled in the art, hybridization can be performed under conditions of various stringency. Suitable hybridization conditions are such that the recognition interaction between a capture sequence and a target nucleic acid is both sufficiently specific and sufficiently stable. Conditions that increase the stringency of a hybridization reaction are widely known and published in the art. See, for example, Green, et al., (2012), infra.

The term “protein” refers to a polymeric form of amino acids of any length, i.e. greater than 2 amino acids, greater than about 5 amino acids, greater than about 10 amino acids, greater than about 20 amino acids, greater than about 50 amino acids, greater than about 100 amino acids, greater than about 200 amino acids, greater than about 500 amino acids, greater than about 1000 amino acids, greater than about 2000 amino acids, usually not greater than about 10,000 amino acids, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence, fusions with heterologous and homologous leader sequences, with or without N-terminal methionine residues; immunologically tagged proteins; fusion proteins with detectable fusion partners, e.g., fusion proteins including as a fusion partner a fluorescent protein, β-galactosidase, luciferase, etc.; and the like. Also included by these terms are polypeptides that are post-translationally modified in a cell, e.g., glycosylated, cleaved, secreted, prenylated, carboxylated, phosphorylated, etc., and polypeptides with secondary or tertiary structure, and polypeptides that are strongly bound, e.g., covalently or non-covalently, to other moieties, e.g., other polypeptides, atoms, cofactors, etc.

The terms “hybridizing” and “binding”, with respect to nucleic acids, are used interchangeably.

It must be noted that as used herein and in the appended claims, the singular forms “a”, “an”, and “the” include plural referents unless the context clearly dictates otherwise, e.g., when the word “single” is used. For example, reference to “an analyte” includes a single analyte and multiple analytes, reference to “a capture agent” includes a single capture agent and multiple capture agents, reference to “a detection agent” includes a single detection agent and multiple detection agents, and reference to “a cell” includes a single cell and multiple cells.

### 1. Rapid Pathology and Cytology without Washing

According to one embodiment of the present invention, a method of stain a tissue, comprising:
(a) placing on a bottom plate a slice of tissue to be stained;
(b) providing a flexible top plate having, on its surface, spacers with a uniform height of 100 um or less;
(c) depositing a stain solution either on the tissue surface or on the flexible top plate; and
(d) sandwiching the sample and the stain solution between the top flexible plate and the bottom plate and pressing them together;

wherein the flexibility of the top flexible plate and the spacing between the spacer are selected to make the top flexible plate conform to the surface of the tissue.

In another embodiment, a method of rapidly analyzing the pathology or cytology of a sample without wash, comprising:
(e) providing a first plate and a second plate; each, on its surface, having a sample contact area for contacting a sample that contains or is suspected of containing a target tissue or cell;
(f) providing a staining solution that stains the target tissue or cell;
(g) sandwiching the sample and the stain solution between the first and the second plates to form a thin layer of thickness of 200 um or less; and
(h) after step (c) and without washing the sample to remove stain solution from the sample, imaging the thin layer to observe the stained target tissue or cell;

wherein the thickness of the thin layer and the concentration of the stain solution in the thin layer are selected to make, during the imaging, the stained target tissue or cell in the thin layer is distinguishable from the rest of thin layer.

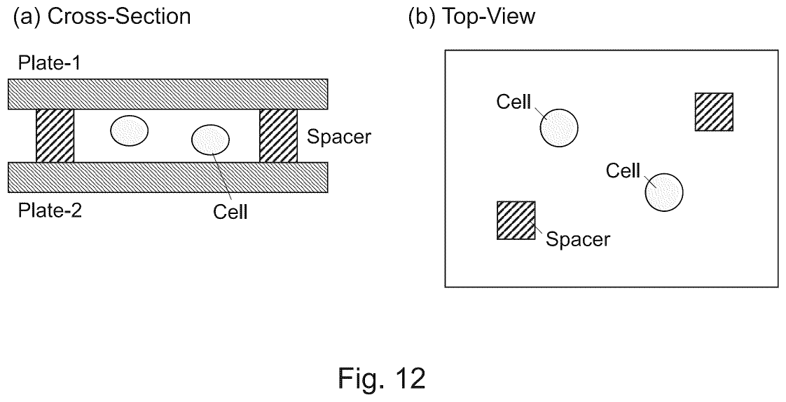

FIG. 12 illustrates of regulating the plate-spacing by putting spacers between the two plates of a sample holder, and the density of the spacer is controlled to have less effects on the cells in the sample that is sandwiches between the plate.

In some embodiments, a kit for analyzing the pathology of a sample without wash, comprising:
(a) a first plate and a second plate; each, on its surface, having a sample contact area for contacting a sample that contains or is suspected of containing a target tissue or cell; and
(b) a staining solution that stains the target tissue or cell;

wherein the first and second plates sandwich the sample and the stain solution into a thin layer of thickness that is regulated by the distance between the two sample contact areas;

wherein the thin layer is imaged by an imager without washing the sample to remove stain solution from the sample; and

wherein the distance between the two sample contact areas and the concentration of the stain solution in the thin layer are selected to make, during the imaging, the stained target tissue or cell in the thin layer is distinguishable from the rest of thin layer.

In some embodiments, an apparatus for analyzing the pathology of a sample without wash, comprising:
(a) a first plate and a second plate; each, on its surface, having a sample contact area for contacting a sample that contains or is suspected of containing a target tissue or cell;
(b) a staining solution that stains the target tissue or cell; and
(c) an imager

wherein the first and second plates sandwich the sample and the stain solution into a thin layer of a thickness that is regulated by the distance between the two sample contact areas;

wherein the imager images the thin layer without washing the sample to remove stain solution from the sample; and

wherein the distance between the two sample contact areas and the concentration of the stain solution in the thin layer are selected to make, during the imaging, the stained target tissue or cell in the thin layer is distinguishable from the rest of thin layer.

Thin layer thickness, incubation time, and background signal. According to the present invention, the thickness of the thin layer for X-Path is selected for several reasons.
(1) A thin thickness of the thin layer makes the staining solution thickness thin, which reduces the diffusion distance for a stain agent in the stain solution to across the thickness, hence reducing the diffusion time. This leads to a short incubation time and saving of the stain agent usage reducing cost.
(2) A thin thickness of the thin layer also reduce the background signal generated by the uncomsumed stain agent in the stain solution. We found experimentally that the thinner the thin layer thickness, the less the background signal, and the clearer the image of the stained cell.

Concentration of stain agent in the stain solution. According to the present invention, the concentration of the stain agent in the stain solution for X-Path is selected, so that, for a given thin layer thickness and at the end of an incubation, most of the stain agent in the stain solution is consumed for staining the target tissue or cell, having little left in the stain solution. This can reduce background signal in imaging and can save the cost on stain agent.

According to the present invention, in some embodiments of assaying (including staining) using a Q-Card, the spacer height is configured to make the assay having a stain saturation time is 5 sec, 10 sec, 20 sec, 30 sec, 60 sec, 90 sec, 120 sec, 180 sec, 300 sec, 600 sec, or a range between any two of the values. In some embodiments, the spacers have a height of 0.5 um, 1 um, 2 um, 5 um, 10 um, 20 um, 30 um, 40 um, 50 um, or a range between any two of the values.

In some preferred embodiments, the a stain saturation time is 5 sec, 10 sec, 20 sec, 30 sec, 60 sec, or a range between any two of the values. In some preferred embodiments, the spacers have a height of 0.5 um, 1 um, 2 um, 5 um, 10 um, 20 um, or a range between any two of the values. In some embodiments, the spacer is 10 um height.

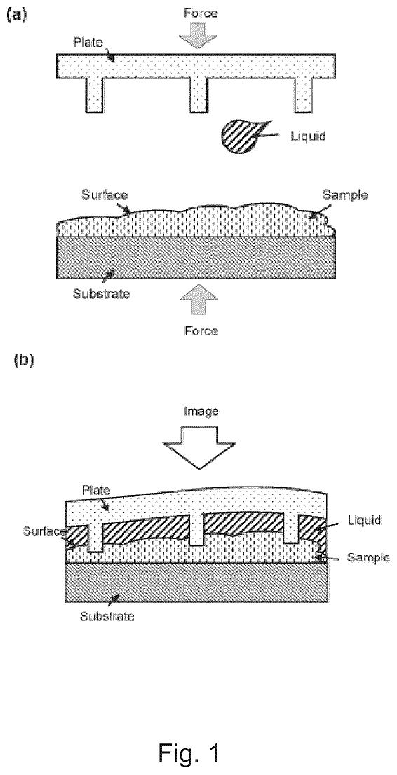

FIG. 1 illustrates an embodiment of the present disclosure, in which a conformable plate comprising spacers conforms to a shape of the surface of a sample.

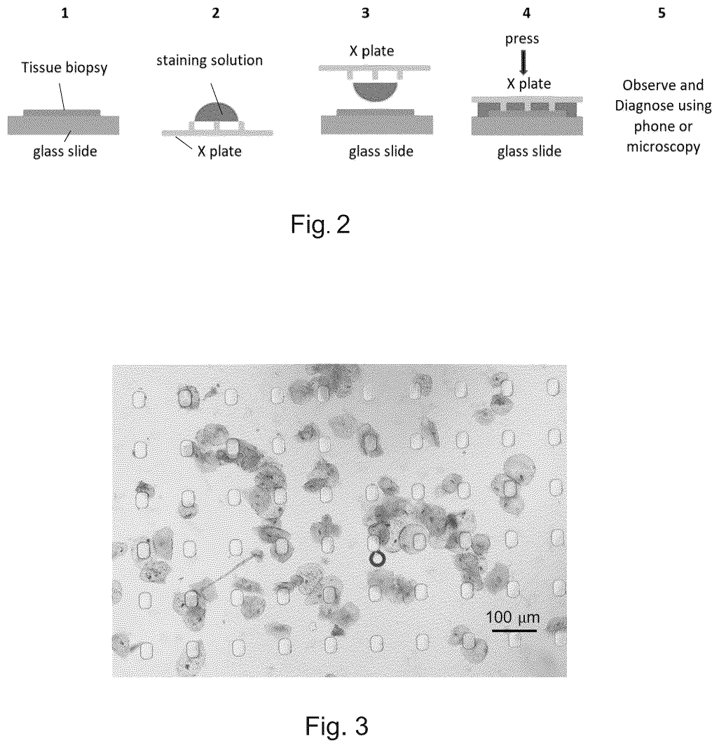

FIG. 2. Schematic figure of experimental procedure using MOST Q-CARD microvolume embodiment. 1, place oral Q-tip brush biopsy or chicken stomach tissue biopsy (5 mm×1 mm) on sub-card or glass slide; 2, drop 10 ul (volume can be varied based on the size of tissue and pillar height) staining solution on the pillar side of X-plate; 3, place X-plate on top of glass slide with staining solution facing down towards tissues; 4, press X-plate onto tissue to allow staining solution mounting the whole piece of tissue, and to spread the tissue into micrometer thin layer (which thickness determined by pillar height); 5, leave MOST Q-Card embodiment at room temperature for 1 min and observe under microscopy. (Note: The Term “Q-CARD” and “QMAX Card” are interchangeable.)

### Example 1 One Step, Fast PAP Staining of Oral Mucosa Cells (OMCs)

X-Path method has been used for Papanicolaou staining (also termed “PAP staining”) Papanicolaou staining solution comprising: hematoxylin, eosin azure (eosin Y, light green SF, Bismarck brown Y), orange green 6, alcohol, and water.

In the experiment, human fresh oral mucosa cells (OMCs) are used to demonstrate the one step, fast PAP staining using Q-Card. The Q-Card used has a pillar array on one of the plate as the spacer, wherein the pillars have uniform height of 10 um, and the pillar array has square lattice with a pillar size of 30 um by 40 um and a period of 110 um by 120 um.

First, the epithelial cells were taken from a subject mouth by a swap. The cells were deposited from the swap to one plate of Q-card (no spacers), forming a thin layer. Then 5-10 ul Papanicolaou staining solution was dropped on the another plate of the Q-card (X-plate having 10 um spacers), followed by pressing two plates together, which sandwich the thin epithelial cell layer and the staining solution and deform them into a uniform 10 um thick thin layer. The thin thickness of the thin layer reduces stain agent diffusion time and makes the staining reaching its saturation in less than 60 seconds, ready for imaging. Then the sample was imaged by an imaging device (e.g. laboratory microscope or smart-phone based microscope).

FIG. 3 Bright field microscopy image of 1 step PAP staining of oral mucosa cells (OMCs) using Q-Card. In the experiment, the Q-Card has a spacer height of 10 um with a pillar array with pillar size of 30 um by 40 um size and period of 110 um by 120 um. The OMC cells are stained to red color in the image. The saturation stain incubation was least 60 second and ready for imaging.

Another example of performing a Pap staining of OMCs using Q-Card, includes steps of:
1. Obtained a Q-Card device;
2. Used an oral swab gently collect cells from month;
3. OMC swab were first mixed with 100 to 200 ul of PBS buffer in collection tube;
4. Mixed 5 uL of OMCs PBS sample with 5 uL of PAP staining solution in collection tube for 1 min;
5. Opened the Q-Card, dropped 10 uL of mixed solution onto Q-Card, closed the Q-Card and observed using bright field imaging system, which includes microscopy and/or smartphone based microscopy system.
6. Images were analyzed using image processing software, and parameters were given to characterize each cell and nuclei, including cell size (C) and number, nuclear size (N) and number, N/C ratio, morphology, architecture distribution, nuclei segmentation and classification.

Another example of performing a Pap staining of OMCs using Q-Card, includes steps of:

1. Obtained a Q-Card device; part of pap staining solution is pre-dried on one of Q-Card plates; 2. Used an oral swab gently collect cells from month; 3. OMC swab were first mixed with buffer in collection tube; 4. Opened the Q-Card, dropped 5-10 ul of OMCs sample in collection tube onto Q-Card, wait 60 seconds, and observed using bright field imaging system, which includes microscopy and/or smartphone based microscopy system. And 5. Images were analyzed using image processing software, and parameters were given to characterize each cell and nuclei, including cell size (C) and number, nuclear size (N) and number, N/C ratio, morphology, architecture distribution, nuclei segmentation and classification.

### Example 2 PAP (Papanicolaou) Staining of Cervix Swap and HPV

Pap staining using X-Path can be used for Pap tests (also termed “Pap smear”), which are tests for finding abnormal cells on patients' cervix that could lead to cervical cancer. Pap tests find cell changes caused by human papillomavirus (HPV). The present invention can make the PAP test's sample staining and preparation in a single step and in about 60 seconds, ready for imaging. The cells can be collected by a swap from a patient's cervix.

### Example 3: TB Diagnostic Using Sputum

One example of the application of the present invention, is for rapid diagnosis of pulmonary tuberculosis. In some embodiments, the devices and the methods of the present invention are used for smear microscopy of sputum to detect Mycobacterium tuberculosis (TB) by detecting the TB cells in sputum or alike samples. The staining used in the present invention include, not limited to: 1) Ziehl-Neelsen staining, or so-called fast acid staining, to stain acids resistant mycolic acids on bacterial wall; 2) fluorescent acid fast staining using auramine-O or auramine-rhodamine, nucleic acid-binding dye SYBR® Gold.

Methodology of conventional smear microscopy detection of TB in sputum includes: 1) Ziehl-Neelsen staining, or so-called fast acid staining, to stain acids resistant mycolic acids on bacterial wall. In principle, this staining method include three major steps: staining, acid differentiation and counter-staining. Whole procedure often takes up to 1 hr with more than 10 steps. 2) fluorescent acid fast staining using auramine-O (AO) or auramine-rhodamine. The standard-AO procedure requires eight steps, three stains, and 22 min to complete (standard-AO stain kit package insert; Remel, Lenexa, KS).

The present method shold shorten Ziehl-Neelsen staining and fluorescent acid fast staining procedures to 1 min as following. Ziehl-Neelsen staining (acid fast staining) of TB.

Material:
1. Raw sputum or digested and decontaminated sputum;
2. Q-Card device;
3. TB staining solution include carbol fuchsin stain, acid alcohol, methylene blue and water;
4. mobile communication device or microscope.

Procedure:
1. Drop 1-5 ul of digested and decontaminated sputum sample onto bottom card of Q-Card device;
2. Drop 1:1 v/v of staining solution onto X-plate of Q-Card;
3. Close Q-Card and leave at room temperature for 1 min;
4. Observe using mobile communication device or microscope.

Results: TB bacterial will show bright-red color and background tissue will show blue color on mobile communication device or microscope images. Quantification and grades of positive staining will be read and output within 1 min on mobile communication device.
1. FAST fluorescent acid fast staining of TB.

Material:
1. Raw sputum or digested and decontaminated sputum;
2. Q-Card device;
3. TB staining solution include auramine rhodamine, acid alcohol, potassium permanganate and water;
4. mobile communication device or fluorescent microscope.

Procedure:
1. Drop 1-5 ul of digested and decontaminated sputum sample onto bottom card of Q-Card device;
2. Drop 1:1 v/v of staining solution onto X-plate of Q-Card;
3. Close Q-Card and leave at room temperature for 1 min;
4. Observe using IPHONE or microscope.

Results: TB bacterial should show fluoresce reddish-orange against a dark background on mobile communication device or fluorescent microscope images. Non-acid-fast organisms will not fluoresce or may appear a pale yellow color on mobile communication device or fluorescent microscope images. Quantification and grades of positive staining will be read and output within 1 min on mobile communication device using software.

Another example is the Influenza Detection from Epithelial Cells using X-PATH and swap sample.

2. Rapid Tissue Sample Pre and Staining by Press Thinning

Another aspect of the present invention is a method of rapidly preparing and staining a tissue sample that has an initial thickness much thicker than the final thickness for staining and imagining analysis. Experimentally, we found that the Q-Card can press a millimeter thick fresh tissue into a film of ˜10 um, and that the spacers of Q-Card can reduce or avoid damages to the cells on the tissue surface. In pressing a tissue sample by a Q-Card, the spacers prevent the top plate (i.e. the plate on the imaging size) to directly contact the tissue surface, and the top surface of a tissue sample is pressed by staining solution, which can even the pressure applied the tissue surface and avoid the “hot pot” (high pressure location) that a solid plate would create. The present invention allows a fresh tissue to be cut, load, stained and imaged in minutes and a few steps without frozen sample, without precision cutting, and without washing.

In one embodiment of the present invention, a method of rapidly preparing and staining a tissue with an initial thickness thicker than the final thickness, comprising:
(a) providing a first plate and a second plate that are movable relative to each other, wherein the second plate has spacers of uniform heights on its surface;
(b) placing, on the first plate, a tissue sample that has a thickness thicker than the spacer height;
(c) depositing a staining solution on the first plate or on the tissue, wherein the staining solution stains the tissue sample;
(d) placing the first plate on top of the tissue sample and pressing to two plates together to make the tissue sample to form a thin layer, wherein the final thickness of the tissue sample layer is less than the initial thickness of the tissue sample and is regulated by the spacers and the two plates.

After the sample preparation and staining, the sample is ready for imaging. In some embodiments, after step (c) and without washing the sample to remove stain solution from the sample, imaging the thin layer to observe the stained tissue, wherein the thickness of the thin layer and the concentration of the stain solution in the thin layer are selected to make, during the imaging, the stained target tissue or cell in the thin layer is distinguishable from the rest of thin layer.

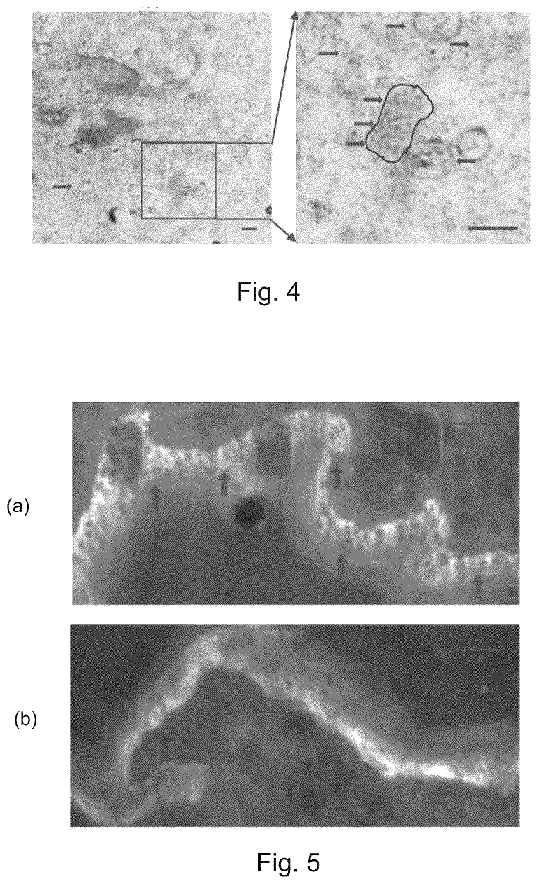

FIG. 4. Chicken stomach mucosa cytology staining by comformal press thinning with hematoxylin. Initially, a thin layer single chicken stomach (epithelial cells) tissue was ˜1 mm thick. By press thinning, the tissue was significantly thinned and some location has nearly the same height as the pillar height (10 um). However, the pressed cells were not damaged. The chicken stomach epithelial cells were hematoxylin positively stained (100×, left panel) with blue/purple color (left panel). With higher magnification, the functional unit, mucosa crypt, was able to be visualized as in the green circle (right panel).

In some embodiments, the final thickness (controlled by the spacer height) of the thin layer sample is selected to make the staining time (the time that a staining reaches saturation) less 60 seconds.

In some embodiments, the time from the completion of the step (d) to imaging is 60 second or less, 120 second or less, 300 second or less, 600 second or less, 900 second or less, or a range between any two of the values.

In some embodiments, the spacers are separated from the both plates, e.g. bead or nanoparticles. In some embodiments, the beads are in the staining solution. In some embodiments, the spacers are pre-fabricated on the surface of the second plate. In some embodiments, the spacers are fabricated on the surface of the second plate and are pillar shape and have a flat top.

In some embodiments, the biopsy sample includes but not limit to exfoliated cell, exfoliated tissue, soft connective tissue, muscle tissue, liver tissue, stomach tissue, small intestine tissue, large intestine tissue, kidney tissue, heart tissue, lung tissue, bone marrow tissue, brain tissue, skin tissue, fat tissue.

In some embodiments, the biopsy sample is imprecisely cut from the biology substance. The imprecise cut means the size, thickness, and dimensions of the sample is not precisely controlled when cutting. In some embodiments, the biopsy sample is fresh without pre-processing as frozen, chemical treatments and others.

In some embodiments, the initial thickness of the tissue sample is 1 um, 5 um, 10 um, 30 um, 50 um, 100 um, 500 um 1000 um, 2000 um, 3000 um, 5000 um, or a range between any two of the values. In some embodiments, the preferred thickness of the tissue sample after cutting is 500 um, 1000 um, 2000 um, 3000 um or a range between any two of the values.

In some embodiments, the size of the tissue sample after cutting is 0.01 mm², 0.1 mm², 0.5 mm², 1 mm², 2 mm², 5 mm², 10 mm², 50 mm², 100 mm², 400 mm², 1000 mm² or a range between any two of the values. In some embodiments, the preferred size of the tissue sample after cutting is 1 mm², 2 mm², 5 mm², 10 mm², 50 mm², 100 mm², 400 mm², 1000 mm² or a range between any two of the values.

In some embodiments, the pressure of the force applied to pressing the two plates to thin the tissue sample has a pressure range of 0.1 kg/cm² to 50 kg/cm². In some embodiments, the pressure has a range of 0.5 kg/cm² to 5 kg/cm². In some embodiments, the pressure has a range of 1 kg/cm² to 3 kg/cm².

In some embodiments, the final thickness of the tissue sample after pressing is 0.1 um, 0.2 um, 0.5 um, 1 um, 5 um, 10 um, 30 um, 50 um, 100 um, 200 um, 500 um or a range between any two of the values. In some embodiments, the final thickness of the tissue sample after pressing is 1 um, 2 um, 3 um, 5 um, 10 um, or a range between any two of the values.

TABLE E1
Examples of tissue thickness change after
pressing in Q-Card with 10 um pillar height

| | Initial Tissue | |
|---|---|---|
| Tissue/organ type | thickness# | Final Tissue thickness |
| exfoliated cell/tissue^{Δ} | 0-3 mm drop | pillar height |
| | or <1 mm smear | |
| soft connective tissue | 1-2 mm | pillar height |
| liver | 1-2 mm | pillar height |
| Stomach | 1-2 mm | pillar height -- ~100 um |
| small intenstine | 1-2 mm | pillar height -- ~100 um |
| large intestine | 1-2 mm | pillar height -- ~100 um |
| kidney | 1-2 mm | pillar height -- ~100 um |
| Muscle | 1-2 mm | pillar height -- ~100 um |
| Heart* | 1-2 mm | pillar height -- ~100 um |
| lung* | 1-2 mm | pillar height -- ~100 um |
| bone marrow* | 0-3 mm drop | pillar height |
| | or <1 mm smear | |
| brain* | 1-2 mm | pillar height |
| skin* | 1-2 mm | pillar height -- ~100 um |
| fat* | 1-2 mm | pillar height |

#from scissors or blade cuts
*estimate number
^{Δ}including needle and endoscopy biopsies

Example of fast staining a biological surface for imaging by using one thin conformable plate and spacers. In some embodiments, only one, but two plates is used, wherein the plate is configured to conformable to the topology of a sample surface, wherein the conformable plate is configured by control the inter-spacer spacing (IDS) and increasing the plate flexibility.

The spacer height is 0.1 um, 0.2 um, 0.3 um, 0.5 um, 1 um, 2 um, 3 um, 5 um, 7 um, 10 um, or a range between any of the two values.

### Examples of Biopsy Sample Thickness, Pressing Force, and Spacer Height:

In some embodiments, the biopsy sample has an original thickness of 10 um to 2000 um, the device has a spacer height of 1 um to 50 um, the pressing force is 0.5 kg/cm² to 20 kg/cm²; after pressing, the biopsy sample has an average thickness of 1 um to 100 um.

In some embodiments, the biopsy sample has an original thickness of 50 um to 1000 um, the device has a spacer height of 5 um to 30 um, the pressing force is 1 kg/cm² to 10 kg/cm²; after pressing, the biopsy sample has an average thickness of 5 um to 50 um.

In some embodiments, the biopsy sample has an original thickness of 100 um to 1000 um, the device has a spacer height of 2 to 10 um, the pressing force is 2 kg/cm² to 5 kg/cm²; after pressing, the biopsy sample has an average thickness of 2 um to 20 um.

In some embodiments, the biopsy sample has an original thickness of 500 um to 2000 um, the device has a spacer height of 2 to 30 um, the pressing force is 1 kg/cm² to 10 kg/cm²; after pressing, the biopsy sample has an average thickness of 2 um to 50 um.

In some embodiments, the biopsy sample has an original thickness of 1000 um to 2000 um, the device has a spacer height of 1 to 100 um, the pressing force is 0.5 kg/cm² to 15 kg/cm²; after pressing, the biopsy sample has an average thickness of 1 um to 150 um.

In some embodiments, the biopsy sample has an original area of 1 mm² to 100 mm², the device has a spacer height of 5 to 50 um, the pressing force is 0.5 kg/cm² to 20 kg/cm²; after pressing, the biopsy sample has an average area of 100 mm² to 1000 mm².

In some embodiments, the biopsy sample has an original area of 0.5 mm² to 10 mm², the device has a spacer height of 2 to 30 um, the pressing force is 1 kg/cm² to 10 kg/cm²; after pressing, the biopsy sample has an average area of 100 mm² to 400 mm².

The height of spacer above the biopsy sample after pressing: In some embodiments, the average height of spacer above the biopsy sample after pressing is 0.1 um, 0.2 um, 0.5 um, 1 um, 5 um, 10 um, 30 um, 50 um, or a range between any two of the values. In some embodiments, the preferred average height of spacer above the biopsy sample after pressing is 1 um, 2 um, 3 um, 5 um, 10 um, or a range between any two of the values.

The Height of Spacer Inside the Biopsy Sample after Pressing:

In some embodiments, the average height of spacer inside the biopsy sample after pressing is 0.1 um, 0.2 um, 0.5 um, 1 um, 5 um, 10 um, 30 um, 50 um, or a range between any two of the values. In some embodiments, the preferred average height of spacer inside the biopsy sample after pressing is 1 um, 2 um, 3 um, 5 um, 10 um, or a range between any two of the values.

The volume of reagent solution before pressing: In some embodiment, no liquid reagent is added into the device. In some embodiment, the staining reagent is printed onto one of the plate of the device. In some embodiment, a liquid reagent is added onto first plate, or biopsy sample or second plate before pressing. In some embodiment, the volume of liquid reagent added into the device is 0 uL, 1 uL, 2 uL, 3 uL, 5 uL, 10 uL, 20 uL, 30 uL, 50 uL or a range between any two of the values.

The thickness of the flexible plate times the Young's modulus (hE): In some embodiment, at least one of the plates is a flexible plate, and the thickness of the flexible plate times the Young's modulus of the flexible plate is in the range of 1 GPa·μm to 1000 GPa·μm.

In some embodiment, at least one of the plates is a flexible plate, and the thickness of the flexible plate times the Young's modulus of the flexible plate is in the range of 10 GPa·μm to 500 GPa·μm.

In some embodiment, at least one of the plates is a flexible plate, and the thickness of the flexible plate times the Young's modulus of the flexible plate is preferred in the range of 20 GPa·μm to 150 GPa·μm.

In some embodiment, at least one of the plates is a flexible plate, and the thickness of the flexible plate times the Young's modulus of the flexible plate is preferred in the range of 1 GPa·μm to 20 GPa·μm.

Spacer height: In some embodiment, the spacer height is 0 um, 1 um, 2 um, 3 um, 5 um, 10 um, 20 um, 30 um, 50 um, 100 um, or a range between any two of the values.

In some embodiment, the preferred spacer height is 2 um, 3 um, 5 um, 10 um, 30 um or a range between any two of the values. In some embodiment, the preferred spacer height is 2 um to 50 um. In some embodiment, the preferred spacer height is 2 um to 30 um. In some embodiment, the preferred spacer height is 5 um to 10 um. In some embodiment, the preferred spacer height is 10 um.

The fourth power of the inter-spacer-distance (ISD) divided by the thickness of the flexible plate (h) and the Young's modulus (E):

In some embodiment, a fourth power of the inter-spacer-distance (IDS) divided by the thickness (h) and the Young's modulus (E) of the flexible plate (ISD⁴/(hE)) is 5×10⁶ um³/GPa or less.

In some embodiment, a fourth power of the inter-spacer-distance (IDS) divided by the thickness (h) and the Young's modulus (E) of the flexible plate (ISD⁴/(hE)) is 1×10⁶ um³/GPa or less.

In some embodiment, a fourth power of the inter-spacer-distance (IDS) divided by the thickness (h) and the Young's modulus (E) of the flexible plate (ISD⁴/(hE)) is 5×10⁵ um³/GPa or less.

The thickness of the flexible plate (h): In some embodiment, wherein the plate is a flexible plate, and the thickness of the flexible plate is 1 um to 500 um.

In some embodiment, wherein the plate is a flexible plate, and the preferred thickness of the flexible plate is 3 um to 175 um.

In some embodiment, wherein the plate is a flexible plate, and the preferred thickness of the flexible plate is 5 um to 50 um.

The Young's modulus (E): In some embodiment, at least one of the plates is a flexible plate, and the Young's modulus of the flexible plate is 0.01 GPa to 100 GPa.

In some embodiment, at least one of the plates is a flexible plate, and the Young's modulus of the flexible plate is 0.1 GPa to 50 GPa.

In some embodiment, at least one of the plates is a flexible plate, and the preferred Young's modulus of the flexible plate is 1 GPa to 5 GPa.

In some embodiment, at least one of the plates is a flexible plate, and the preferred Young's modulus of the flexible plate is 0.01 GPa to 1 GPa.

The imaging system: In some embodiment, the imaging system detect signal from sample includes but not limit to photoluminescence, electroluminescence, and electrochemiluminescence, light absorption, reflection, transmission, diffraction, scattering, or diffusion, surface Raman scattering, electrical impedance selected from resistance, capacitance, and inductance, magnetic relativity and a combination thereof.

In some embodiment, the imaging system is a microscope, a bright field microscope, phase contrast microscope, fluorescence microscope, inverted microscope, the compound light microscope, stereo microscope, digital microscope, acoustic microscope, phone based microscope.

### The Analyzing System:

In some embodiment, the analyzing system includes but not limit to machine learning, supervised machine learning, unsupervised machine learning, and reinforcement learning. In some embodiment, the analyzing system combines both the software analyzing and human analyzing.

### 3. Tissue Imaging Improvement Using Conformable Flexible Plate (i.e. Film) of Uniform Micro-Spacers

As shown in FIG. 1, after a sample is deposited onto a substrate, a liquid is deposited onto one or both of the sample and the plate, which can comprise spacers. Upon pressing the plate together with the substrate (e.g., with the sample sandwiched therebetween), the liquid is compressed into a layer having uniform thickness, wherein the thickness of the layer is regulated by the height of the spacers. Furthermore, as the plate is flexible (e.g., conformable), the plate conforms to any irregularities in the shape of the sample, thereby maintaining a uniformly thick layer of liquid above the sample.

We found that tissue imaging improvement using conformable flexible plate (i.e. film) of uniform micro-spacers.

### Example 4: Imaging Improvement

FIG. 5 illustrates a comparison between fluorescence images obtained with (i) one-step staining using an X-plate with the spacers contacting the tissue, and (ii) using flat palte. The image using X-plate have better image contrast and sharper image than that of the flat plate.

Human frozen skin sections were first stained with anti-CK14-AF488 using 10 um pillar height X-plate. Tissue sections were then recovered with PBS by using X-plate (10 um pillar height, top panel) or flat PMMA film (top panel). Observe tissue sections under fluorescent microscope. Red arrows indicate CK14-AF488 positive skin epithelial cells. 10 uM pillar X-plate shows better imaging result comparing with PMMA film.

Materials. Fresh human skin and lung cryo-sections are from Zyagen, tissue sections are stored at −80° C. before use. Antibodies used in this study are all commercially prelabelled with Alexa Flour 488 (AF488). Alexa Fluor family of fluorescent dyes is a series of dyes invented by Molecular Probes, now a part of Thermo Fisher Scientific, and sold under the Invitrogen brand name. Alexa Fluor dyes are frequently used as cell and tissue labels in fluorescence microscopy and cell biology. Alexa Fluor dyes can be conjugated directly to antibodies to amplify signal and sensitivity or other biomolecules. Commercial prelabelled antibodies used in this study are anti-cytokeratin 14-AF488 (from Novus Biologicals, cat no. NBP2-34675AF488), and palloidin-AF488 (for detection of F-actin, from Thermo Fisher Scientific, cat no. A12379). All antibodies are saved at 4° C. protective from light before use. A cell membrane permeable dye acridine orange (AO, from Sigma Aldrich, cat no. A9231) is also used in the study to visualize nuclear under fluorescent microscope. X plates are manufactured in Essenlix Coop. X-Plate is 175 um thick PMMA with a pillar array of 30 um×40 um pillar size, 5 um pillar height and 80 um inter space distance.

It is one aspect of the present disclosure to provide easy and rapid devices and methods for tissue staining. In some embodiments, the reduction of the thickness of the staining liquid significantly reduces the time of staining agent(s) to diffuse across the thickness of the staining liquid, hence decreasing the saturation time for whatever purposes the staining agent(s) is for. For instance, such a configuration decreases the saturation time for antigen-antibody binding, which is the speed-limiting step of immunostaining, by reducing the diffusion distance of the antibody used for the staining, greatly promoting the overall speed for immunostaining.

It is another aspect of the present disclosure to provide uniform access to staining agent for the sample using the devices and methods for tissue staining disclosed therein. In some embodiments, the uniform thickness of the staining liquid engendered by the particular configuration of the plates and spacers ensures the uniform access of the sample to the staining agent that is dissolved and diffuses in the staining liquid.

Sample

It should be noted that, the term “sample” as used herein, unless otherwise specified, refers to a liquid bio/chemical sample or a non-liquid sample.

In some embodiments, the liquid sample is originally obtained in a liquid form, such as, blood and saliva. In some embodiments, the originally obtained sample specimen is not in a liquid state, for instance, in a solid state or a gaseous state. In such cases, the non-liquid sample is converted to a liquid form when being collected and preserved using the device and method provided by the present disclosure. The method for such conversion includes, but not limited to, mixture with a liquid medium without dissolution (the end product is a suspension), dissolution in a liquid medium, melting into a liquid form from a solid form, condensation into a liquid form from a gaseous form (e.g. exhaled breath condensate).

In some embodiments, the sample can be dried thereon at the open configuration, and wherein the sample comprises bodily fluid selected from the group consisting of: amniotic fluid, aqueous humour, vitreous humour, blood (e.g., whole blood, fractionated blood, plasma or serum), breast milk, cerebrospinal fluid (CSF), cerumen (earwax), chyle, chime, endolymph, perilymph, feces, breath, gastric acid, gastric juice, lymph, mucus (including nasal drainage and phlegm), pericardial fluid, peritoneal fluid, pleural fluid, pus, rheum, saliva, exhaled breath condensates, sebum, semen, sputum, sweat, synovial fluid, tears, vomit, urine, and any combination thereof.

In some embodiments, the sample contact area of one or both of the plates is configured such that the sample can be dried thereon at the open configuration, and the sample comprises blood smear and is dried on one or both plates.

In some embodiments, the sample is a solid sample, for instance, a tissue section. In some embodiments, the sample is a solid tissue section having a thickness in the range of 1-200 μm. In some embodiments, the sample contact area of one or both of the plates is adhesive to the sample. In some embodiments, the sample is paraffin-embedded. In some embodiments, the sample is fixed (e.g., formalin, paraformaldehyde and the like).

Staining Liquid

In some embodiments, the staining liquid has a viscosity in the range of 0.1 to 3.5 mPa S.

In some embodiments, one primary function of the staining liquid is to serve a transfer medium. The reagents stored (dried/coated) on the plate(s), upon contacting the staining liquid, are dissolved and diffuse in the staining liquid. As such, the staining liquid serves as a transfer medium to provide access for the reagents stored on the plate(s) to the sample.

In some embodiments, one primary function of the staining liquid is to serve as a holding solution. When the plates are pressed to enter the closed configuration, in some embodiments, the plates are configured to “self-hold” at closed configuration after the removal of the external compressing force, due to forces like capillary force provided by the liquid sample. In the cases where the sample specimen is not in a liquid form, the liquid medium therefore provides such forces like capillary force needed for the “self-holding” of the plates.

In some embodiments, the staining liquid comprises buffer pairs to balance the pH value of the final solution. In some embodiments, the staining liquid does not comprise particular component capable of altering the properties of the sample.

In some embodiments, the staining liquid comprises reagents needed for the processing, fixation, or staining of the sample, as further discussed in details in the following sections.

In some embodiments, the staining liquid comprises fixative capable of fixing the sample.

In some embodiments, the staining liquid comprises blocking agents, wherein the blocking agents are configured to disable non-specific endogenous species in the sample to react with detection agents that are used to specifically label the target analyte.

In some embodiments, the staining liquid comprises deparaffinizing agents capable of removing paraffin in the sample.

In some embodiments, the staining liquid comprises permeabilizing agents capable of permeabilizing cells in the tissue sample that contain the target analyte.

In some embodiments, the staining liquid comprises antigen retrieval agents capable of facilitating retrieval of antigen.

In some embodiments, the staining liquid comprises detection agents that specifically label the target analyte in the sample.

Plate Storage Site

In some embodiments, the sample contact area of one or both plates comprise a storage site that contains reagents needed for the processing, fixation, or staining of the sample. These reagents, upon contacting the liquid sample or the staining liquid, are dissolved and diffuse in the liquid sample/staining liquid.

In some embodiments, the sample contact area of one or both plates comprise a storage site that contains blocking agents, wherein the blocking agents are configured to disable non-specific endogenous species in the sample to react with detection agents that are used to specifically label the target analyte.

In some embodiments, the sample contact area of one or both plates comprise a storage site that contains deparaffinizing agents capable of removing paraffin in the sample.

In some embodiments, the sample contact area of one or both plates comprise a storage site that contains permeabilizing agents capable of permeabilizing cells in the tissue sample that contain the target analyte.

In some embodiments, the sample contact area of one or both plates comprise a storage site that contains antigen retrieval agents capable of facilitating retrieval of antigen.

In some embodiments, the sample contact area of one or both plates comprise a storage site that contains detection agents that specifically label the target analyte in the sample.

In some embodiments, the sample contact area of one or both of the plates comprise a binding site that contains capture agents, wherein the capture agents are configured to bind to the target analyte on the surface of cells in the sample and immobilize the cells.

Detection Agent

In some embodiments, the detection agent comprises dyes for a stain selected from the group consisting of: Acid fuchsin, Alcian blue 8 GX, Alizarin red S, Aniline blue WS, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, C.I. Cresyl violet, Crystal violet, Darrow red, Eosin B, Eosin Y, Erythrosin, Ethyl eosin, Ethyl green, Fast green F C F, Fluorescein Isothiocyanate, Giemsa Stain, Hematoxylin, Hematoxylin & Eosin, Indigo carmine, Janus green B, Jenner stain 1899, Light green SF, Malachite green, Martius yellow, Methyl orange, Methyl violet 2B, Methylene blue, Methylene blue, Methylene violet, (Bernthsen), Neutral red, Nigrosin, Nile blue A, Nuclear fast red, Oil Red, Orange G, Orange II, Orcein, Pararosaniline, Phloxin B, Protargol S, Pyronine B, Pyronine, Resazurin, Rose Bengal, Safranine O, Sudan black B, Sudan Ill, Sudan IV, Tetrachrome stain (MacNeal), Thionine, Toluidine blue, Weigert, Wright stain, and any combination thereof.

In some embodiments, the detection agent comprises antibodies configured to specifically bind to protein analyte in the sample.

In some embodiments, the detection agent comprises oligonucleotide probes configured to specifically bind to DNA and/or RNA in the sample.

In some embodiments, the detection agent is labeled with a reporter molecule, wherein the reporter molecule is configured to provide a detectable signal to be read and analyzed.

In some embodiments, the reporter molecule comprises fluorescent molecules (fluorophores), including, but not limited to, IRDye800CW, Alexa 790, Dylight 800, fluorescein, fluorescein isothiocyanate, succinimidyl esters of carboxyfluorescein, succinimidyl esters of fluorescein, 5-isomer of fluorescein dichlorotriazine, caged carboxyfluorescein-alanine-carboxamide, Oregon Green 488, Oregon Green 514; Lucifer Yellow, acridine Orange, rhodamine, tetramethylrhodamine, Texas Red, propidium iodide, JC-1 (5,5′,6,6′-tetrachloro-1,1′,3,3′-tetraethylbenzimidazoylcarbocyanine iodide), tetrabromorhodamine 123, rhodamine 6G, TMRM (tetramethyl rhodamine methyl ester), TMRE (tetramethyl rhodamine ethyl ester), tetramethylrosamine, rhodamine B and 4-dimethylaminotetramethylrosamine, green fluorescent protein, blue-shifted green fluorescent protein, cyan-shifted green fluorescent protein, redshifted green fluorescent protein, yellow-shifted green fluorescent protein, 4-acetamido-4′-isothiocyanatostilbene-2,2′disulfonic acid; acridine and derivatives, such as acridine, acridine isothiocyanate; 5-(2′-aminoethyl)aminonaphthalene-1-sulfonic acid (EDANS); 4-amino-N-[3-vinylsulfonyl)phenyl]naphth-alimide-3,5 disulfonate; N-(4-anilino-1-naphthyl)maleimide; anthranilamide; 4,4-difluoro-5-(2-thienyl)-4-bora-3a,4a diaza-5-indacene-3-propioni-c acid BODIPY; cascade blue; Brilliant Yellow; coumarin and derivatives: coumarin, 7-amino-4-methylcoumarin (AMC, Coumarin 120),7-amino-4-trifluoromethylcoumarin (Coumarin 151); cyanine dyes; cyanosine; 4′,6-diaminidino-2-phenylindole (DAPI); 5′,5″-dibromopyrogallol sulfonaphthalein (Bromopyrogallol Red); 7-diethylamino-3-(4′-isothiocyanatophenyl)-4-methylcoumarin; diethylenetriaamine pentaacetate; 4,4′-diisothiocyanatodihydro-stilbene-2-,2′-disulfonic acid; 4,4′-diisothiocyanatostilbene-2,2′-disulfonic acid; 5-(dimethylamino]naphthalene-1-sulfonyl chloride (DNS, dansylchloride); 4-dimethylaminophenylazophenyl-4′-isothiocyanate (DABITC); eosin and derivatives: eosin, eosin isothiocyanate, erythrosin and derivatives: erythrosin B, erythrosin, isothiocyanate; ethidium; fluorescein and derivatives: 5-carboxyfluorescein (FAM),5-(4,6-dichlorotriazin-2-yl)amino-fluorescein (DTAF), 2′,7′dimethoxy-4′5′-dichloro-6-carboxyfluorescein (JOE), fluorescein, fluorescein isothiocyanate, QFITC, (XRITC); fluorescamine; IR144; IR1446; Malachite Green isothiocyanate; 4-methylumbelliferoneortho cresolphthalein; nitrotyrosine; pararosaniline; Phenol Red; B-phycoerythrin; ophthaldialdehyde; pyrene and derivatives: pyrene, pyrene butyrate, succinimidyl 1-pyrene; butyrate quantum dots; Reactive Red 4 (Cibacron™ Brilliant Red 3B-A) rhodamine and derivatives: 6-carboxy-X-rhodamine (ROX), 6-carboxyrhodamine (R6G), lissamine rhodamine B sulfonyl chloride rhodamine (Rhod), rhodamine B, rhodamine 123, rhodamine X isothiocyanate, sulforhodamine B, sulforhodamine 101, sulfonyl chloride derivative of 5 sulforhodamine (Texas Red); N,N,N′,N′-tetramethyl-6-carboxyrhodamine (TAMRA); tetramethyl rhodamine; tetramethyl hodamine isothiocyanate (TRITC); riboflavin; 5-(2′-aminoethyl) aminonaphthalene-1-sulfonic acid (EDANS), 4-(4′-dimethylaminophenylazo)benzoic acid (DABCYL), rosolic acid; CAL Fluor Orange 560; terbium chelate derivatives; Cy 3; Cy 5; Cy 5.5; Cy 7; IRD 700; IRD 800; La Jolla Blue; phthalo cyanine; and naphthalo cyanine, coumarins and related dyes, xanthene dyes such as rhodols, resorufins, bimanes, acridines, isoindoles, dansyl dyes, aminophthalic hydrazides such as luminol, and isoluminol derivatives, aminophthalimides, aminonaphthalimides, aminobenzofurans, aminoquinolines, dicyanohydroquinones, fluorescent europium and terbium complexes; combinations thereof, and the like. Suitable fluorescent proteins and chromogenic proteins include, but are not limited to, a green fluorescent protein (GFP), including, but not limited to, a GFP derived from Aequoria victoria or a derivative thereof, e.g., a “humanized” derivative such as Enhanced GFP; a GFP from another species such as Renilla reniformis, Renilla muller, or Ptilosarcus guernyi; “humanized” recombinant GFP (hrGFP); any of a variety of fluorescent and colored proteins from Anthozoan species; any combination thereof; and the like.

In some embodiments, the signal is selected from the group consisting of:
i. luminescence selected from photo-luminescence, electroluminescence, and electro-chemiluminescence;
ii. light absorption, reflection, transmission, diffraction, scattering, or diffusion;
iii. surface Raman scattering;
iv. electrical impedance selected from resistance, capacitance, and inductance;
v. magnetic relaxivity; and
vi. any combination of i-v.

### Immunohistochemistry

In some embodiments, the devices and methods of the present disclosure are useful for conducting immunohistochemistry on the sample.

In immunohistochemical (IHC) staining methods, a tissue sample is fixed (e.g., in paraformaldehyde), optionally embedding in wax, sliced into thin sections that are less then 100 μm thick (e.g., 2 μm to 6 μm thick), and then mounted onto a support such as a glass slide. Once mounted, the tissue sections may be dehydrated using alcohol washes of increasing concentrations and cleared using a detergent such as xylene. In certain cases, fixation is also an optional step, for instance, for blood smear staining.

In most IHC methods, a primary and a secondary antibody may be used. In such methods, the primary antibody binds to antigen of interest (e.g., a biomarker) and is unlabeled. The secondary antibody binds to the primary antibody and directly conjugated either to a reporter molecule or to a linker molecule (e.g., biotin) that can recruit reporter molecule that is in solution. Alternatively, the primary antibody itself may be directly conjugated either to a reporter molecule or to a linker molecule (e.g., biotin) that can recruit reporter molecule that is in solution. Reporter molecules include fluorophores (e.g., FITC, TRITC, AMCA, fluorescein and rhodamine) and enzymes such as alkaline phosphatase (AP) and horseradish peroxidase (HRP), for which there are a variety of fluorogenic, chromogenic and chemiluminescent substrates such as DAB or BCIP/NBT.

In direct methods, the tissue section is incubated with a labeled primary antibody (e.g. an FITC-conjugated antibody) in binding buffer. The primary antibody binds directly with the antigen in the tissue section and, after the tissue section has been washed to remove any unbound primary antibody, the section is to be analyzed by microscopy.

In indirect methods, the tissue section is incubated with an unlabeled primary antibody that binds to the target antigen in the tissue. After the tissue section is washed to remove unbound primary antibody, the tissue section is incubated with a labeled secondary antibody that binds to the primary antibody.

After immunohistochemical staining of the antigen, the tissue sample may be stained with another dye, e.g., hematoxylin, Hoechst stain and DAPI, to provide contrast and/or identify other features.

The present device may be used for immunohistochemical (IHC) staining a tissue sample. In these embodiments, the device may comprise a first plate and a second plate, wherein: the plates are movable relative to each other into different configurations; one or both plates are flexible; each of the plates has, on its respective surface, a sample contact area for contacting a tissue sample or a IHC staining liquid; the sample contact area in the first plate is smooth and planner; the sample contact area in the second plate comprise spacers that are fixed on the surface and have a predetermined substantially uniform height and a predetermined constant inter-spacer distance that is in the range of 7 μm to 200 μm;

wherein one of the configurations is an open configuration, in which: the two plates are completely or partially separated apart, the spacing between the plates is not regulated by the spacers; and wherein another of the configurations is a closed configuration which is configured after a deposition of the sample and the IHC staining liquid in the open configuration; and in the closed configuration: at least part of the sample is between the two plates and a layer of at least part of staining liquid is between the at least part of the sample and the second plate, wherein the thickness of the at least part of staining liquid layer is regulated by the plates, the sample, and the spacers, and has an average distance between the sample surface and the second plate surface is equal or less than 250 μm with a small variation.

As discussed above, in some embodiments, the device may comprise a dry IHC staining agent coated on the sample contact area of one or both plates. In some embodiments, the device may comprise a dry IHC staining agent coated on the sample contact area of the second plate, and the IHC staining liquid comprise a liquid that dissolve the dry IHC staining agent. In some embodiments, the thickness of the sample is 2 μm to 6 μm.

H&E, Special Stains, and Cell Viability Stains

In some embodiments, the devices and methods of the present disclosure are useful for conducting H&E stain, special stains, and cell viability stains.

Hematoxylin and eosin stain or haematoxylin and eosin stain (H&E stain or HE stain) is one of the principal stains in histology. It is the most widely used stain in medical diagnosis and is often the gold standard; for example when a pathologist looks at a biopsy of a suspected cancer, the histological section is likely to be stained with H&E and termed “H&E section”, “H+E section”, or “HE section”. A combination of hematoxylin and eosin, it produces blues, violets, and reds.

In diagnostic pathology, the “special stain” terminology is most commonly used in the clinical environment, and simply means any technique other than the H&E method that is used to impart colors to a specimen. This also includes immunohistochemical and in situ hybridization stains. On the other hand, the H&E stain is the most popular staining method in histology and medical diagnosis laboratories.

In any embodiments, the dry binding site may comprise a capture agent such as an antibody or nucleic acid. In some embodiments, the releasable dry reagent may be a labeled reagent such as a fluorescently-labeled reagent, e.g., a fluorescently-labeled antibody or a cell stain such Romanowsky's stain, Leishman stain, May-Grunwald stain, Giemsa stain, Jenner's stain, Wright's stain, or any combination of the same (e.g., Wright-Giemsa stain). Such a stain may comprise eosin Y or eosin B with methylene blue. In certain embodiments, the stain may be an alkaline stain such as haematoxylin.

In some embodiments, the special stains include, but not limited to, Acid fuchsin, Alcian blue 8 GX, Alizarin red S, Aniline blue WS, Auramine O, Azocarmine B, Azocarmine G, Azure A, Azure B, Azure C, Basic fuchsine, Bismarck brown Y, Brilliant cresyl blue, Brilliant green, Carmine, Chlorazol black E, Congo red, C.I. Cresyl violet, Crystal violet, Darrow red, Eosin B, Eosin Y, Erythrosin, Ethyl eosin, Ethyl green, Fast green F C F, Fluorescein Isothiocyanate, Giemsa Stain, Hematoxylin, Hematoxylin & Eosin, Indigo carmine, Janus green B, Jenner stain 1899, Light green SF, Malachite green, Martius yellow, Methyl orange, Methyl violet 2B, Methylene blue, Methylene blue, Methylene violet, (Bernthsen), Neutral red, Nigrosin, Nile blue A, Nuclear fast red, Oil Red, Orange G, Orange II, Orcein, Pararosaniline, Phloxin B, Protargol S, Pyronine B, Pyronine, Resazurin, Rose Bengal, Safranine O, Sudan black B, Sudan Ill, Sudan IV, Tetrachrome stain (MacNeal), Thionine, Toluidine blue, Weigert, Wright stain, and any combination thereof.

The term “cell viability stains” refers to staining technology used to differentially stain live cells and dead cells inside a tissue sample. Usually the difference in cell membrane and/or nucleus membrane permeability between live and dead cells are taken advantage for the differential staining. In other cases, markers for apoptosis or necrosis (indicating dying cells or cell corpses) are used for such staining.

In some embodiments, the device comprises, on one or both of the plates, a dye to stain the sample for cell viability. In some embodiments, the dye includes, but not limited to, Propidium Iodide (PI), 7-AAD (7-Aminoactinomycin D), Trypan blue, Calcein Violet AM, Calcein AM, Fixable Viability Dye (FVD) conjugated with different fluorophores, SYTO9 and other nucleic acid dyes, Resazurin and Formazan (MTT/XTT) and other mitochondrial dyes, and any combination thereof and the like. In some embodiments, the sample comprises bacteria, and it is desirable to determine the bacterial viability in the sample, the device further comprises, on one or both of the plates, a bacterial viability dye, for instance, PI, SYTO9, and the like, to differentially stain the live cells versus dead cells. Optionally, the device further comprises, on one or both of the plates, dyes for total bacterial staining, for instance, gram staining reagents and the like.

In Situ Hybridization

In some embodiments, the devices and methods of the present disclosure are useful for conducting in situ hybridization (ISH) on histological samples.

In situ hybridization (ISH) is a type of hybridization that uses a labeled complementary DNA, RNA or modified nucleic acids strand (i.e., probe) to localize a specific DNA or RNA sequence in a portion or section of tissue (in situ), or, if the tissue is small enough (e.g., plant seeds, Drosophila embryos), in the entire tissue (whole mount ISH), in cells, and in circulating tumor cells (CTCs).

In situ hybridization is used to reveal the location of specific nucleic acid sequences on chromosomes or in tissues, a crucial step for understanding the organization, regulation, and function of genes. The key techniques currently in use include: in situ hybridization to mRNA with oligonucleotide and RNA probes (both radio-labelled and hapten-labelled); analysis with light and electron microscopes; whole mount in situ hybridization; double detection of RNAs and RNA plus protein; and fluorescent in situ hybridization to detect chromosomal sequences. DNA ISH can be used to determine the structure of chromosomes. Fluorescent DNA ISH (FISH) can, for example, be used in medical diagnostics to assess chromosomal integrity. RNA ISH (RNA in situ hybridization) is used to measure and localize RNAs (mRNAs, lncRNAs, and miRNAs) within tissue sections, cells, whole mounts, and circulating tumor cells (CTCs).

In some embodiments, the detection agent comprises nucleic acid probes for in situ hybridization staining. The nucleic acid probes include, but not limited to, oligonucleotide probes configured to specifically bind to DNA and/or RNA in the sample.

Systems and Methods for Tissue Staining and Cell Imaging

Also provided is a system for rapidly staining and analyzing a tissue sample using a mobile phone, comprising:
(a) sample, staining liquid, and device as described above,
(b) a mobile communication device comprising:
i. one or more cameras for detecting and/or imaging the sample;
ii. electronics, signal processors, hardware and software for receiving and/or processing the detected signal and/or the image of the sample and for remote communication; and
(c) a light source from either the mobile communication device or an external source.

Also provided is a method for rapidly staining and analyzing a tissue sample using a mobile phone, comprising:
(a) depositing a tissue sample and a staining liquid on the device of the system described above, and placing the two plate into a closed configuration;
(b) obtaining a mobile phone that has hardware and software of imaging, data processing, and communication;
(c) assaying by the tissue sample deposited on the CROF device by the mobile phone to generate a result; and
(d) communicating the result from the mobile phone to a location remote from the mobile phone.

### 4. Rapid Homogenous Intracellular Assay

Most of the assays today (except pathology and cytology) detect a biomarker related to a disease or disorder in bio system by detecting the biomarker in a sample while the biomarker is outside a cell (e.g. the biomarker leaks out from a cell into a sample or a cell in a sample is lysed). Often the concentration of biomarker outside a cell may be low that makes a detection of the biomarker challenging and/or complicated.

One aspect of the present invention is to detect a biomarker while the biomarker is still inside a cell. One reason for such approach is that the concentration of a biomarker in a cell is generally much higher than that when the biomarker comes out the cell into the sample. For example, for a biomarker originally in lymphocyte, the concentration of the biomarker in lymphocyte can be ˜5,000 times higher than that if the lymphocyte is lysed and the biomarker is mixed in the blood. Such high biomarker concentration cell can greatly facilitate the biomarker detection.

In some embodiments, for a sample that contains cells and an analyte both outside of the cell (cell free) and inside the sample, the concentration of a cell free (outside a cell) analyte in sample is measured through measuring the signal of the analyte inside the cells. In some embodiments, the signal of the analyte inside cell is generated by using a probe to bind the analyte inside of the cell. The relationship between the concentration of the cell-free analyte in a sample and the signal of the analyte inside a cell is established by measuring the cell-free analyte in the sample, measuring the signal of the analyte inside cell of the sample. In some embodiments, the probe binds the analyte specifically. In some embodiments, the probe generates fluorescent light.

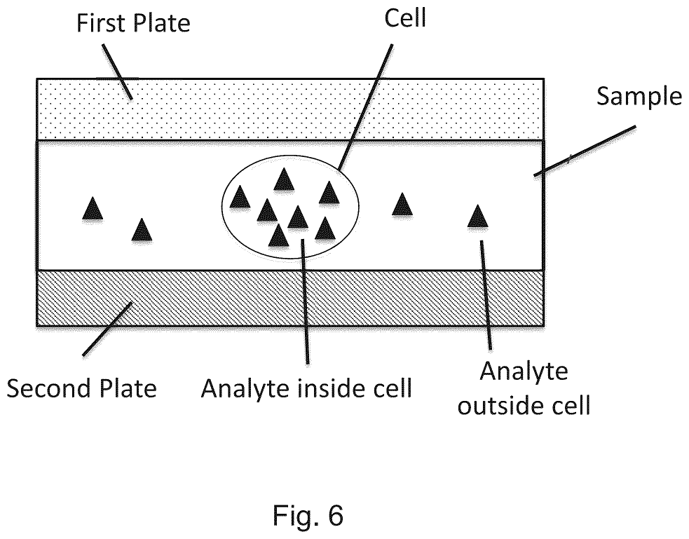

FIG. 6. Illustration of intra-cellular assay that determining the concentration of a cell free analyte in a sample using the signal of the analyte inside the cell. A sample with a cell sandwiched between two plates, wherein the cell contains analytes (i.e. biomarkers) inside of the cell, and some of analytes are outside the cell. In some embodiments, the analyte concentration inside the cell has a colleration with that outside the cell.

One aspect of the present invention is to detect a biomarker in a cell for detecting a disease or disorder in a bio system, by introducing a labeled probe into a cell, wherein the labeled probe specifically bines to the biomarker.

One aspect of the present invention, the labeled probe comprises protein, nucleic acids, aptamer, or a combination thereof.

AA. In some embodiments, a method for a rapid homogenous detection of an analyte inside a membrane of a cell in a sample, comprising:
(a) providing a first plate and a second plate, each, on its surface, having a sample contact area for contacting a sample comprising a cell that contains or is suspected of containing an analyte inside the cell,
(b) providing a detection probe that (i) specifically binds the analyte and (ii) is capable of emitting a light at a wavelength;
(c) providing a permeabilization reagent that makes a membrane of the cell permeable to the detection probe, wherein without the permeabilization reagent the detection probe cannot permeate into the cell;
(d) sandwiching the sample, the detection probe, and the permeabilization reagent between the first and second plates to form a thin layer of a thickness of 200 microns (um) or less; and
(e) after the step (d) and without washing the sample to remove unbound detection probe, imaging the thin layer to detect the cell that has the analyte bound to the detection probe;
wherein the thickness of the thin layer and the concentration of the detection probe in the thin layer are selected to make, in the thin layer, the signal from the location having the detection probe bound to the analyte inside the cell distinguishable from signals from the locations that do not have the cell during the imaging of step (e).

The method of embodiment sAA, the imaging of step (e) is performed 300 seconds or less after sandwiching of step (d).

The method of embodiments AA, wherein the thickness of the thin layer is selected to make some of the cell having no overlap or significant overlap with other cells in the thin layer.

The method of embodiments AA, further comprising a step of quantifying (i) the cell that has an analyte inside the cell and (ii) the cell that does have an analyte inside the cell.

The method of embodiments AA, further comprising a step of quantifying (i) the cell that has an analyte inside the cell and (ii) the cell that does have an analyte inside the cell, and a step of quantifying the percentage of the cell having an analyte inside the cell relative to the total number of the cell.

The method of embodiments AA, wherein the light emitted by the detection probe is fluorescence, and wherein the method further comprises (i) measuring the fluorescence intensity of the cell having the analyte bound to the detection probe, (ii) measuring the number of the cells having the analyte bound to the detection probe, and (iii) calculating a total fluorescence intensity by multiplying the total number of cells having the analyte bound to the detection probe in a unit area and the average of the fluorescence intensity of the cell having the analyte bound to the detection probe.

AB In some embodiments, a method of determining, for a sample contains cells and an analyte inside and outside the cell, the concentration of the analyte that ouride a cell in the sample using a signal of the analyte inside a cell of the sample, comprising:
(a) obtain a sample comprising a cell and an analyte both outside and inside the cell;
(b) obtaining a sample holder comprising a first plate and a second plate, which the plates can be configured to face each other and sandwich the sample into a thickness determined by the plate-spacing;
(b) depositing the sample between the plates;
(c) having reagents for staining and penetration of the cell;
(d) having an imager;
(e) imaging the sample and analyze the signal of the analyte inside the cell; and
(f) determining the concentration of the analyte outside the cell of the sample using a relationship between the signal of the analyte inside of the cell and the concentration of the analyte outside the cell of the sample.

In some embodiments, the signal of the analyte inside cell of the sample comprising: Nt: total number of pictured cells, Np (number of positively stained cells), % Np/Nt (percentage of Np over Nt), Fn (Fluorescent intensity from each pictured cell), MF (mean of positive fluorescent intensity from positively stained cells), TF (total positive fluorescent intensity by multiplying MF with % Np/Nt.), or any combination of thereof.

In some embodiments, the relationship is established before the step (e).

In some embodiments, a method for quantifying a cell-free biomaker in a whole blood, comprising:
(a) having a blood sample that contains and is suspected of containing the cell-free biomarker;
(b) detecting and quantifying the biomaker inside a cell in the whole blood by specific intra-cellular protein immune-detection;
(c) detecting and counting the cells that contain the biomarker;
(d) calculating a total signal by multiplying the detected signal of the biomarker in each cell (detected and quantified in step (b)) by the total number of the cells that contain the biomarker (detected and counted in step (c)); and
(e) related the total signal to the concentration of the biomarker free in the whole blood.

In some embodiments, a method for INSH images analysis, comprising:
a. having a whole blood sample that contains or is suspected of containing a biomaker;
b. performing specific intra-cellular RNA hybridization detection to a labeled RNA detection agent to specifically hybridize the RNA related to the biomaker, wherein the detection comprising imaging using an imager (e.g. microscope);
c. opening microscope images by an image software.
d. Obtaining average fluorescent signals of each cells from the image and background signals(noise);
e. Calculating signal (S) to noise(N) ratio by using formula: (S−N)/N for each images;
h. Relating the normalized signal with the cell-free biomarker concentration in the whole blood.

In some embodiments, a method of quantification of intracellular protein expression level using ISIM, comprising
a. having a whole blood sample that contains or is suspected of containing a biomaker;
b. performing specific intra-cellular protein immuno detection to a labeled protein detection agent to specifically bind to the biomaker, wherein the detection comprising imaging using an imager;
c. after 1 min staining of intracellular protein using ISIM;
d. Images are then analyzed and reported the parameters comprising: Nt: total number of pictured cells, Np: number of positively stained cells, % Np/Nt: percentage of Np over Nt, Fn: Fluorescent intensity from each pictured cell, MF: Mean of positive fluorescent intensity from positively stained cells, and TF: total positive fluorescent intensity by multiplying MF with % Np/Nt. 20.

In some embodiments, the intracellular stain formulation comprises an intracellular stain reagent containing a viral probe molecule [e.g., p24 protein or p24 mRNA]; a buffer; and a cell permeabilizer.

In some embodiments, the intracellular stain formulation comprises a fluorescent-labeled oligo nucleotide probe.

In some embodiments, the at least one disease diagnosis is selected from: a blood cancer, an infectious disease, an autoimmune disease, a primary immunodeficiency (PID), a genetic disease, a benign urinary tract disease or condition, a urinary tract cancer, or a malignant disease.

In some embodiments, the method further comprising reporting the at least one disease diagnosis remotely with a communication device.

In some embodiments, the intracellular stain formulation comprises an intracellular stain reagent containing a probe molecule; a buffer; and a cell permeabilizer.

In some embodiments, the sample comprises a single cell.

In some embodiments, the sample comprises whole blood.

In some embodiments, at least one cell comprises a white blood cell, a red blood cell, a granulocyte, or a combination thereof.

In some embodiments, contacting the sample with the formulation and the resulting chemical interaction with the biomarker is accomplished in a single step.

In some embodiments, wherein at least one of:
contacting the sample with the stain formulation; and
the resulting chemical interaction or incubation of the stain formulation with the biomarker for imaging
are accomplished in 60 seconds or less.

In some embodiments, contacting the sample with the formulation and the resulting chemical interaction with the biomarker is accomplished in a single step.

In some embodiments, at least one of:
contacting the sample with the stain formulation;
the resulting chemical interaction or incubation of the stain formulation with the biomarker; imaging; or
analyzing the image, is accomplished in 60 seconds or less.

In some embodiments, the sample is a fresh crude biological sample selected from a needle biopsy, whole blood, urine, sputum, saliva, a swab sample (e.g., a pap smear), sweat, breath, breast milk, bile, or results from pathological process (such as blister or cyst fluid).

In some embodiments, the presence of the targeted intracellular biomarker is indicative of the presence of at least one disease.

In some embodiments, the presence and quantity of the targeted intracellular biomarker is more indicative than not of the presence of at least one disease.

In some embodiments, the presence and quantity of the targeted intracellular biomarker is more indicative of the at least one disease and provides at least one disease diagnosis selected from the database of correlated biomarker and disease combinations.

In some embodiments, the biomarker is indicative of at least one disease selected from an infectious disease, malignant disease, autoimmune disease, a metabolic disease, an inherited genetic disorder disease; or a combination thereof.

In some embodiments, the specific intra-cellular protein is cytokine.

In some embodiments, the specific intra-cellular protein comprises. Infectious disease and antibiotic resistance biomarkers.

In some embodiments, the labeled RNA detection agent is mRNA.

In some embodiments, the specific intra-cellular protein is IL-6 and anti-human IFN-γ.

In some embodiments, the intracellular stain formulation comprises an intracellular stain reagent containing an antibody probe molecule [e.g., AF488-anti-IL-4, and AF647-anti-IL6 antibodies] and/or oligonucleotide probe molecule [e.g., IL-6 Alexa488 60-mer oligo probe, SEQ. ID #1]; a buffer; and a cell permeabilizer.

In some embodiments, the reagents, a labeled RNA detection agent, and/or specific intra-cellular protein are coated one or both of the plates.

### Example 25 Viral Detection

According to the present invention, a method of a detection of a viral infection of a subject comprising: detecting a virus specific glycoprotein inside a cell of the subject (i.e. glycoprotein inside a membrane of a cell), wherein a virus glycoprotein specific probe is introduced inside a cell (e.g. inside plasma and/or nucleus), wherein the probe has a label, wherein the probe concentration in the cell is configured so that at least one portion of the volume of the cell has a concentration of the probes bound to a specific virus glycol-protein higher than that of unbind probes in a portion of the volume of (i) other portion of the cell and/or (ii) other portion of a volume outside of the cell. In some embodiments, the staining reagent may comprise a permeabilizing agent capable of permeabilizing cells in the tissue sample that contain the target analyte.

In some embodiments, the analyte is glycol-protein of virus.

In some embodiments, the spacers are separated from the both plates, e.g. bead or nanoparticles. In some embodiments, the beads are in the staining solution. In some embodiments, the spacers are pre-fabricated on the surface of the second plate. In some embodiments, the spacers are fabricated on the surface of the second plate and are pillar shape and have a flat top.

In some embodiments, staining solution include: PH7 to PH8 buffer for antibody staining, such as PBS, Saline, Tris buffer, HEPES buffer, sodium bicarbonate.

In some embodiments, staining solution include detergent such as: 1-6% Zwittgent

In some embodiments, a permearization of cell is by:
i. Organic solvent based solutions: 50%-70% ethanol or isopropyl alcohol in buffer;
ii. Nanoparticle carriers to deliver antibody into white blood cells, such as lipid or polymetric based nanoparticles;
iii. Electroporation to deliver antibody into white blood cells;
iv. Peptide mediated antibody delivery into white blood cells; or
v. any combination of above.

Example of FAST staining and imaging of virus in white blood cells from whole blood patent material.

Sample collection:
1. Massage to warm the finger and increase blood flow by gently squeezing from hand to fingertip 5-6 times. Wipe dry with clean gauze or allow to air dry.
2. Using a sterile lancet, make a skin puncture just off the center of the finger pad.
3. Wipe away the first drop of blood. Finger smear 1-5 ul of second drop of whole blood directly onto Q-Card.

Staining and Imaging:
1. Mix blood with staining solution including PBS, Zwittgent, ethanol and fluorophore labelled antibody.
2. Close Q-Card and incubate blood sample with staining solution at room temperature for less than 1 min without any washing;
3. After 2, Image white blood cells in the closed Q-Card using PHONE or fluorescent microscope.

Exemplary embodiments of Q-Card, pillar size 0.1-2 um, 2-10 um, 10-30 um.

Exemplary embodiments of Imaging devices: PHONE or fluorescent microscope

Exemplary embodiments in Staining and Imaging:

Exemplary embodiments in Sampling: any biological samples containing virus:
a. blood specimens
b. respiratory specimens
c. cutaneous specimens
d. cervical specimens
e. stool specimens
f. urine specimens
g. Cerebrospinal fluid specimens
h. Any other fine needle specimens, such as amniotic fluid

Examples of Virus can be detected by the present invention include, not limited to:
a. CMV, hepatitis B virus, hepatitis C, EBV and HIV in blood specimen
b. HSV and VZV in cutaneous specimens
c. Rotavirus in stool specimens
d. RSV, influenza and Parainfluenza viruses, and Adenovirus in respiratory specimens
e. HPV in cervical and cutaneous specimens
f. HSV in Cerebrospinal fluid specimens
g. Zika virus in amniotic fluid specimens
h. Coronal virus in various samples (nose swap, mouth swap, saliva)

### Biomarks and Applications

Further aspects of the present disclosure include a CROF device that includes a plurality of capture agents that each binds to a plurality of analytes in a sample, i.e., a multiplexed CROF device. In such instances, the CROF device containing a plurality of capture agents can be configured to detect different types of analytes (protein, nucleic acids, antibodies, etc.). The different analytes can be distinguishable from each other on the array based on the location within the array, the emission wavelength of the detectable label that binds to the different analytes, or a combination of the above.

Other pathogens that can be detected in a diagnostic sample using the devices, systems and methods in the present invention include, but are not limited to: Varicella zoster; Staphylococcus epidermidis, Escherichia coli, methicillin-resistant Staphylococcus aureus (MSRA), Staphylococcus aureus, Staphylococcus hominis, Enterococcus faecalis, Pseudomonas aeruginosa, Staphylococcus capitis, Staphylococcus warneri, Klebsiella pneumoniae, Haemophilus influenzae, Staphylococcus simulans, Streptococcus pneumoniae and Candida albicans; gonorrhea (Neisseria gorrhoeae), syphilis (Treponena pallidum), clamydia (Clamyda tracomitis), nongonococcal urethritis (Ureaplasm urealyticum), chancroid (Haemophilus ducreyi), trichomoniasis (Trichomonas vaginalis); Pseudomonas aeruginosa, methicillin-resistant Staphylococcus aureus (MSRA), Klebsiella pneumoniae, Haemophilis influenzae, Staphylococcus aureus, Stenotrophomonas maltophilia, Haemophilis parainfluenzae, Escherichia coli, Enterococcus faecalis, Serratia marcescens, Haemophilis parahaemolyticus, Enterococcus cloacae, Candida albicans, Moraxiella catarrhalis, Streptococcus pneumoniae, Citrobacter freundii, Enterococcus faecium, Klebsella oxytoca, Pseudomonas fluorscens, Neiseria meningitidis, Streptococcus pyogenes, Pneumocystis carinii, Klebsella pneumoniae Legionella pneumophila, Mycoplasma pneumoniae, and Mycobacterium tuberculosis, etc., as well as those listed in Tables B2 and 6.

Below is a list of diseases and the Diagnostic Markers associated with them:
Alzheimer's disease: AP42, amyloid beta-protein (CSF), prion protein (CSF), proapoptotic kinase R (PKR) and its phosphorylated PKR (pPKR) (CSF)
multiple sclerosis: fetuin-A (CSF), niemann-pick type C: tau (CSF), bipolar disorder: secretogranin II (CSF), prion disease: prion protein (CSF)
HIV-associated neurocognitive disorders: Cytokines (CSF)
Parkinsonian disorders (neuordegenerative disorders): Alpha-synuclein (CSF), tau protein (CSF), Apo H (CSF), ceruloplasmin (CSF), Peroxisome proliferator-activated receptor gamma coactivator-1 alpha (PGC-1a)(CSF)
axonal degeneration: neurofilament light chain (CSF)
neuordegenerative disorders: parkin (CSF), PTEN induced putative kinase 1 (CSF), DJ-1 (CSF), leucine-rich repeat kinase 2 (CSF)
Kufor-Rakeb disease: mutated ATP13A2 (CSF)
CSF rhinorrhea (nasal surgery samples): transthyretin (CSF)
Multiple Sclerosis Progression: Vitamin D-binding Protein (CSF), CXCL13 (CSF)
intrathecal inflammation: IL-12p40, CXCL13 and IL-8 (CSF)
prostate cancer: Dkk-3 (semen)
Sepsis (Endocan, specifically secreted by activated-pulmonary vascular endothelial cells, is thought to play a key role in the control of the lung inflammatory reaction): p14 endocan fragment (blood)
neuromyelitis optica: Serum (blood)
cardiovascular disease: ACE2 (blood), alpha-amylase (saliva)
early diagnosis of esophageal squamous cell carcinoma: autoantibody to CD25 (blood)
lung cancer: hTERT (blood), CA125 (MUC 16) (blood): VEGF (blood), slL-2 (blood),
Osteopontin (blood), BRAF, CCNI, EGRF, FGF19, FRS2, GREB1, and LZTS1 (saliva)
ovarian cancer: Human epididymis protein 4 (HE4) (blood), CA 125 (saliva)
pregnancy: Alpha-Fetal Protein (blood)
diabetics: Albumin (urine)
albuminuria: albumin (urine) uria
kidney leaks: microalbuminuria
mirror fetal AFP levels: AFP (urine)
Acute kidney injury: neutrophil gelatinase-associated lipocalin (NGAL) (urine), interleukin 18 (IL-18) (urine), Kidney Injury Molecule-1 (KIM-1) (urine), Liver Fatty Acid Binding Protein (L-FABP) (urine)
Epstein-Barr virus oncoprotein (nasopharyngeal carcinomas): LMP1 (saliva), BARF1
(saliva)
oral cancer: IL-8 (saliva)
oral or salivary malignant tumors: carcinoembryonic antigen (CEA) (saliva)
Malignant tumors of the oral cavity: carcinoembryonic antigen (saliva)
spinalcellular carcinoma: IL8 (saliva), thioredoxin (saliva)
HIV: beta-2 microglobulin levels—monitor activity of the virus (saliva), tumor necrosis factor-alpha receptors—monitor activity of the virus (saliva)
breast cancer: CA15-3 (saliva)

In some instances, the present method is used to inform the subject from whom the sample is derived about a health condition thereof. Health conditions that may be diagnosed or measured by the present method, device and system include, but are not limited to: chemical balance; nutritional health; exercise; fatigue; sleep; stress; prediabetes; allergies; aging; exposure to environmental toxins, pesticides, herbicides, synthetic hormone analogs; pregnancy; menopause; and andropause. The following Table B3 provides a list of biomarker that can be detected using the present invention, and their associated health conditions.

Oral squamous cell carcinoma: p53

Head and neck squamous cell carcinoma: CASP-8, SART-1, TREX1, 3′ repair exonuclease; BRAP (BRCA1 associated): Nuclear localization protein; Trim 26 zinc finger domains; GTF21 transcription factor. Murine homolog TF11-1; NSEP1 (YB-1) transcription factor; MAZ transcription factor associated with c-myc; SON (DBP-5; KIAA1019; NREBP DNA binding protein); NACA nascent polypeptide-associated complex; NUBP2 nucleotide binding protein; EEF2 Translation elongation factor 2; GU2 Putative RNA helicase; RPLI3A ribosomal protein; SFRS21P (CASP11; SIP1; SRRP1290 splicing factor); RPS12 ribosomal protein; MGC2835 RNA helicase; TMF1, TATA modulatory factor; PRC1 regulator of cytokinesis; KRT14 keratin 14; Viniculin; H2AFY histone family member; SLK (KIAA02304) Ste related kinase; NOL3 (ARC) nuclear protein 3, apoptosis repressor; DNAJA2 member of Hsp40 family; DNAJA1 member of HSP40 family; LINE-1 retrotransposon; MOG (HSPC 165) Homolog of yeast protein; LIMS1 (PINCH): LIM and senescent antigen-like domain; COPB2 coatomer protein complex subunit protein; FLJ22548 hypothetical protein; C21orf97; FLJ21324; MGC15873; SSNA1 Sjogrens syndrome nuclear autoantigen 1; KIAA0530, zinc finger domain; rat stannin; hypothetical protein DKFZp4340032; human FLJ23089; PC326

### 1. Anionic Detergent

Alkyl Sulfates: Lithium dodecyl sulfate, Lithium dodecyl sulfate, Lithium dodecyl sulfate, Niaproof®, Sodium 2-ethylhexyl sulfate, Sodium dodecyl sulfate, Sodium octyl sulfate, Teepol™ 610 S anionic, Turkey red oil sodium salt Alkyl Sulfonates: 1-Octanesulfonic acid sodium salt, 4-Dodecylbenzenesulfonic acid, Ethanesulfonic acid sodium salt, Sodium 1-butanesulfonate, Sodium 1-decanesulfonate, Sodium 1-heptanesulfonate, Sodium 1-nonanesulfonate, Sodium 1-octanesulfonate, Sodium 1-pentanesulfonate, Sodium 1-propanesulfonate, Sodium hexanesulfonate, Sodium pentanesulfonate

Bile Salts: Chenodeoxycholic acid diacetate methyl ester, Chenodeoxycholic acid, Cholic acid, Deoxycholic acid, Glycocholic acid hydrate, Sodium chenodeoxycholate, Sodium cholate hydrate, Sodium cholate hydrate, Sodium cholate hydrate, Sodium cholesteryl sulfate, Sodium deoxycholate, Sodium glycochenodeoxycholate, Sodium glycocholate, Sodium glycodeoxycholate, Sodium taurochenodeoxycholate, Sodium taurocholate, Sodium taurodeoxycholate, Sodium taurohyodeoxycholate, Sodium taurolithocholate, Sodium tauroursodeoxycholate, Taurocholic acid sodium salt, Taurolithocholic acid 3-sulfate disodium salt, Ursodeoxycholic acid

Other Anionic Detergents: Dicyclohexyl sulfosuccinate sodium salt, Dihexadecyl phosphate, Dihexyl sulfosuccinate sodium salt, Docusate sodium, Lithium 3,5-diiodosalicylate, N-Lauroylsarcosine sodium salt, N-Lauroylsarcosine, N-Lauroylsarcosine purum, Sodium octanoate, Triton™ QS-15

### 2. Cationic Detergents

Alkyltrimethylammonium bromide, Amprolium hydrochloride, Benzalkonium chloride, Benzethonium hydroxide, Benzyldimethyldodecylammonium, Benzyldimethylhexadecylammonium, Benzyldodecyldimethylammonium, Cetylpyridinium chloride, Dimethyldioctadecylammonium, Dodecylethyldimethylammonium, Dodecyltrimethylammonium, Ethylhexadecyldimethylammonium bromide, Girard's reagent T, Hexadecyl(2-hydroxyethyl)dimethylammonium dihydrogen phosphate, Hexadecylpyridinium bromide, Hexadecylpyridinium chloride, Hexadecyltrimethylammonium, Luviquat™ FC 370, Luviquat™ FC 550, Luviquat™ HOLD, Luviquat™ Mono LS, Methylbenzethonium chloride, Myristyltrimethylammonium, Tetraheptylammonium bromide, Tetrakis(decyl)ammonium bromide, Tri-C8-10-alkylmethylammonium chloride, Tridodecylmethylammonium chloride Selectophore™

### 3. Non-Ionic Detergent

1-Oleoyl-rac-glycerol, 2-Cyclohexylethyl β-D-maltoside, 4-Nonylphenyl-polyethylene glycol non-ionic, 5-Cyclohexylpentyl β-D-maltoside, 6-Cyclohexylhexyl β-D-maltoside, n-Dodecanoylsucrose, n-Dodecyl-β-D-glucopyranoside, n-Dodecyl-β-D-maltoside, n-Nonyl-β-D-glucopyranoside, n-Octyl-β-D-thioglucopyranoside, n-Decanoylsucrose, n-Decyl-3-D-maltopyranoside, n-Octanoylsucrose, n-Octyl-β-D-glucopyranoside, APO-10, APO-12, BRIJ® 020, BRIJ® 35, Big CHAP, Deoxy, Brij® 58, Brij® L23, Brij® L4, Brij® 010, Cremophor EL®, C₁₂E8, C₁₂E9, DGEA, Decaethylene glycol mono-dodecyl ether nonionic surfactant, Decyl β-D-glucopyranoside, Decyl β-D-maltopyranoside, Decyl-β-D-1-thiomaltopyranoside, Decyl-β-D-maltoside, Diethylene glycol, Digitonin, Digitoxigenin, ELUGENT™ Detergent, Ethylene glycol, GC Stationary Phase phase Synperonic PE/F68, GC Stationary Phase phase Synperonic PE/L64, GENAPOL® X-100, Genapol® C-100, Genapol® X-080, Genapol® X-100, Glucopone 600 CS UP, HECAMEG®, Hexaethylene glycol monododecyl ether, Hexaethylene glycol monohexadecyl ether, Hexaethylene glycol monotetradecyl ether, Hexyl β-D-glucopyranoside, IGEPAL® CA-630, IGEPAL® CA-720, IPTG, Imbentin AGS/35, Isopropyl β-D-1-thiogalactopyranoside, Kolliphor® EL, Lutrol® OP 2000 non-ionic, Methoxypolyethylene glycol 350, Methyl 6-O—(N-heptylcarbamoyl)-α-D-glucopyranoside, N,N-Bis[3-(D-gluconamido)propyl]deoxycholamide, N,N-Dimethyldecylamine N-oxide, N,N-Dimethyldodecylamine N-oxide, N-Decanoyl-N-methylglucamine, N-Lauroyl-L-alanine, N-Nonanoyl-N-methylglucamine, N-Octanoyl-N-methylglucamine, NP-40 Alternative, Nonaethylene glycol monododecyl ether nonionic surfactant, Nonidet™ P 40, Nonyl β-D-glucopyranoside, Nonyl β-D-maltoside, Nonyl-β-D-1-thiomaltoside, Nonylphenyl-polyethyleneglycol acetate, Octaethylene glycol monodecyl ether, Octaethylene glycol monododecyl ether, Octaethylene glycol monohexadecyl ether, Octyl β-D-1-thioglucopyranoside, Octyl β-D-glucopyranoside, Octyl-α/β-glucoside, Octyl-β-D-glucopyranoside, PLURONIC® F-127, Pentaethylene glycol monodecyl ether, Pluronic® F-127, Poloxamer 188, Poloxamer 407, Poly(ethylene glycol) methyl ether, Polyoxyethylene (10) tridecyl ether mixture of C11 to C14 iso-alkyl ethers, Polyoxyethylene (20) sorbitan monolaurate, Polyoxyethylene (40) stearate, Polysorbate 20, Polysorbate 60, Polysorbate 80, SODOSIL™ RM 003, SODOSIL™ RM 01, diethylene glycol octadecyl ether, Saponin, Span® 20, Span® 60, Span® 65, Span® 80, Span® 85, Sucrose monodecanoate, Sucrose monolaurate, Synperonic® F 108, Synperonic® PE P105, TERGITOL™ TMN 10, TERGITOL™ TMN 6, TERGITOL™ solution Type NP-40, TERGITOL™ MIN FOAM, TERGITOL™ Type 15-S-5, TERGITOL™ Type 15-S-7, TERGITOL™ Type 15-S-9, TERGITOL™ Type NP-10, TERGITOL™ Type NP-9, TRITON® X-100, TRITON® X-114, TWEEN® 20, TWEEN® 40, TWEEN® 60, TWEEN® 65, TWEEN® 80, TWEEN®85, Tetradecyl-β-D-maltoside, Tetraethylene glycol monododecyl ether, Tetraglycol, Tetramethylammonium hydroxide pentahydrate, Thesit®, Tridecyl β-D-maltoside, Triethylene glycol monodecyl ether, Triton™ N-57, Triton™ N-60, Triton™ X-100, Triton™ X-102, Triton™ X-114, Triton™ X-165, Triton™ X-305, Triton™ X-405, Triton™ X-45, Tween® 20, Tween® 40, Tween® 60, Tween® 80, Tween® 85, Tyloxapol, Undecyl β-D-maltoside, n-Dodecyl β-D-glucopyranoside, n-Dodecyl β-D-maltoside, n-Heptyl β-D-glucopyranoside, n-Heptyl β-D-thioglucopyranoside, n-Hexadecyl β-D-maltoside, n-Octyl β-D-maltoside

4. Zwitterionic (Ampholytic)

3-(4-tert-Butyl-1-pyridinio)-1-propanesulfonate, 3-(N,N-Dimethylmyristylammonio)propanesulfonate, 3-(N,N-Dimethyloctadecylammonio)propanesulfonate, 3-(N,N-Dimethyloctylammonio)propanesulfonate, 3-(N,N-Dimethylpalmitylammonio)propanesulfonate, 3-(1-Pyridinio)-1-propanesulfonate, 3-(Benzyldimethylammonio)propanesulfonate, 3-(Decyldimethylammonio)-propane-sulfonate inner salt, 3-[N,N-Dimethyl(3-palmitoylaminopropyl)ammonio]-propanesulfonate, L-α-Lysophosphatidylcholine from Glycine max (soybean), ASB-14, zwitterionic amidosulfobetaine. ASB-16 zwitterionic amidosulfobetaine detergent, ASB-C80, ASB-C80, C7BzO, CHAPS, CHAPSO, DDMAB, Dimethylethylammoniumpropane, EMPIGEN® BB Detergent, Miltefosine, N-Dodecyl-N,N-dimethyl-3-ammonio-1-propanesulfonate, N-Dodecyl-N,N-dimethyl-3-ammonio-1-propanesulfonate, O-(Decylphosphoryl)choline, Poly(maleic anhydride-alt-1-decene), 3-(dimethylamino)-1-propylamine derivative, Poly(maleic anhydride-alt-1-tetradecene), 3-(dimethylamino)-1-propylamine derivative, Sodium 2,3-dimercaptopropanesulfonate, Surfactin from Bacillus subtilis, ZWITTERGENT® 3-08, ZWITTERGENT® 3-10, ZWITTERGENT® 3-12, ZWITTERGENT® 3-14, ZWITTERGENT® 3-16,

Below is a list of organic solvent can be used to permeabilize cell membrane: Hydrocarbons: n-Pentane, n-Hexane, n-Heptane, n-Octane, n-Nonane, n-Decane, 2,2,4-Trimethyl pentane, Cyclohexane, Benzene, Toluene, Ethyl benzene, Xylene (mixed isomers), C9 Aromatics, Tetralin

Alcohols: Methano,l Ethano,l n-Propanol, i-Propanol, n-Butanol, i-Butanol, s-Butanol, n-Amyl alcohol, i-Amyl alcohol, Cyclohexanol, n-Octanol, Ethanediol, Diethylene glycol, 1,2-Propanediol

Glycol ethers: Propylene glycol methyl ether, Ethylene glycol methyl ether, Ethylene glycol ethyl ether, Ethylene glycol monobutyl ether

Chlorinated solvents: Methylene chloride, Chloroform, Carbon tetrachloride, 1,2-Dichloroethane, 1,1,1-Trichloroethane, Trichloroethylene, Perchloroethylene, Monochlorobenzen

Ketones: Acetone, Methyl ethyl ketone, Methyl isobutyl ketone, Cyclohexanone, n-Methyl-2-pyrrolidone, Acetophenone

Ethers: Diethyl ether, Diisopropyl ether, Dibutyl ether, Methyl tert butyl ether, 1,4-Dioxane, Tetrahydrofuran

Esters: Methyl acetate, Ethyl acetate, Isopropyl acetate, n-Butyl acetate, Cellosolve acetate

Miscellaneous solvents: Dimethylformamide, Dimethylacetamide, Dimethylsulphoxide, Sulfolane, Carbon disulphide, Acetic acid, Aniline, Nitrobenzene, Morpholine, Pyridine, 2-Nitropropane, Acetonitrile, Furfuraldehyde, Phenol, Water

### Nanoparticles for Intracellular Delivery

1. Inorganic Nanomaterials: gold, silver, calcium phosphate, graphene oxide, quantum dots, and magnetic nanomaterials such as iron oxides. Graphene oxide
2. Carbon Nanotubes (CNTs): multiwalled carbon nanotubes (MWNTs), single walled carbon nanotubes (SWNTs), Polyethylenimine(PEI)-MWNTs, PEI-cholesterol-MWNTs, Succinated PEI (PEI-SA)-CNTs, chitosan-folic acid nanoparticles (CS-FA NPs), functionalized carboxylated MWNTs (fCNT),
3. Proteins and Peptide Nanomaterials
4. Polymer-Based Nanomaterials
5. Lipid-based nanomaterials, see liposomes
6. Liposomes

Various Lipids and Amphiphiles that are Used as Liposome Raw Materials

| Natural | Phosphotidylcholine, Phosphotidylserine, |
|---|---|
| phospholipids | Phosphotidylethanolamine |
| Synthetic | 1,2-Dilauroyl-sn-Glycero-3-Phosphocoline |
| phospholipids | (DLPC), 1,2-Dioleoyl-sn-Glycero-3-[Phospho-L- |
| | Serine] (Sodium Salt) (DOPS), |
| | Dipalmitoylphosphotidylcholine, |
| | Dipalmitoylphosphotidylseine, |
| | Distearoylphosphotidylcholine, |
| | Dipalmitoylphosphotidylglycerol, 1,2- |
| | Dilauroyl-sn-Glycero-3-Phosphocholine (DLPC) |
| Unsaturated | 1-Stearoyl-2-Linoleoyl-sn-Glycero-3-[Phospho- |
| | L-Serine] (Sodium Salt), |
| | Dioleaylphosphotidylcholine |
| Sphingolipids | Shingomyellin |
| Glycosphingolipids | Gangliosides |
| Steroids | Cholesterol |
| Polymeric material | Lipids conjugated to diene, methacrylate & thiol |
| | group |
| Charge-inducing | Diotadecyldimethyl ammonium bromide/chloride |
| lipids | (DODAB/C); Dioleoyl trimethylammonium |
| | propane (DOTAP) |

Types of Liposomes:
Liposomes, Archaeosomes, Niosomes, Novasomes, Cryptosomes, Emulsomes, Vesosomes
7. Polyethyleneglycol (PEG)
8. Polyethyleneimine (PEI)
9. Natural Polymer-Based Nanomaterials

E. Examples of Applications of Present Invention in Diagnosis of Diseases and Disorders

E-1. Diagnosis of Diseases Associated with Infectious Diseases, Including Bacterial, Viral, Fungal, and Parasitic Infections

The devices and the methods in the present invention can be used for diagnosing diseases, including Acinetobacter baumannii, Acinetobacter infections, Actinomyces gerencseriae, Actinomyces israelii, Actinomycosis, Aids, alphaviruses, Amebiasis, Amoebic dysentery, Anabaena, Anaemia, Anaplasma genus, Anaplasmosis, mold spores including Aspergillius Flavis, anthrax, Anthrax, Aphanizomenon, Arcanobacterium haemolyticum infection, Arenaviruses, Argentine hemorrhagic fever, Arsenicosis, ascariasis, Ascariasis, Ascaris lumbricoides, Aspergillius glaucus, Aspergillius niger, Aspergillosis, Aspergillus genus, Astroviridae family Babesiosis, Astrovirus infection, avian influenza, Bacillus anthracis, Bacillus magaterium sp. (Veg), Bacillus magaterium sp. (Spores), Bacillus paratyphusus, Bacillus subtilis, Bacterial vaginosis (BV), Bacteriophage (E. coli), Bacteroides infection, blue-green algae, Bolivian hemorrhagic fever, Botulinum Toxin, Botulism, Brazilian hemorrhagic fever, Brucella, brucellosis, bubonic pague, Bunyaviridae family, Burkholderia mallei, Burkholderia pseudomallei, Buruli ulcer Mycobacterium, Caliciviridae family, Campylobacteriosis, Candidiasis (Moniliasis; Thrush), castor beans, chagas disease, Chlamydia, Chlamydia psittaci, Chlamydophila pneumonia, Chlamydophila pneumoniae infection, Cholera, Clostridium botulinum, Clostridium difficile infection, Clostridium perfringens, Clostridium tetani (Tetanus/Lockjaw), coliform bacteria, Coxiella burnetii, Crimean-Congo haemorrhagic fever, Cryptosporidium, Cryptosporidium parvun, cyanobacterial toxins, Cylindroapermopsis raciborski, Cylindrospermopis, Dengue, diphtheria, E. coli O157:H7, E. Coli, eastern equine encephalitis, Eberthella typosa, Ebola virus, Entamoeba histolytica, Epsilon toxin, Escherichia coli O157:H7, Fluorosis, Francisella tularensis, Giardia Lamblia, Glanders, gonorrhea, Guanarito virus, H1N1, H5N1, hantavirus, Hendra Virus, Hepatitis, Hepatitis A Virus, Hepatitis B Virus, Hepatitis C Virus, Hepatitis D Virus, Hepatitis E Virus, Herpes simplex virus 1 and 2 (HSV-1 and HSV-2), HIV, hookworm disease otitis media, human monkeypox, Influenza (Flu), Japanese Encephaltis, junin virus, Lassa fever, Lassa virus, legionellosis, leishmaniasis, Lenionella, leprosy, Leptospira Canicoal-infections (Jaundice), Leptospirosis, lymphatic filariasis, Machupo virus, Malaria, Marburg haemorrhagic fever, Marburg virus, measles, Melioidosis, meningitis, meningococcal disease, Methaemoglobinemia, Methicillin-resistant Staphylococcus Aureus (MRSA) Micrococcus candidus, Micrococcus spheroids, microcystin, Mucor racemosus A, Mucor racemosus B, Mycobacterium tuberculosis (Tuberculosis), Neisseria catarrhalis, Nipah virus, Nodularia, Nostoc, Onchocerciasis, Oospora lactis, Oscillatoria, Paratyphoid enteric fevers, Penicillium digitatum, Penicillium expansum, Penicillium roqueforti, pertussis, Phtomomnas aeruginosa, plague, poliomyelitis, Poliovirus-Poliomyelitis, Propionibacterium propionicus, Pseudomonas fluorescens, Psittacosis, Q fever, respiratory infections, Rhisophus nigricans, Ricin toxin, Ricinus communis, Rickettsia prowazekii, rift valley fever, Ringworm, Rotovirus, Sabia, Salmonella enteritidis, Salmonella paratyphi (Enteic Fever), Salmonella species, Salmonella Typhi, Salmonella typhimurium, Salmonella typhosa (Typhoid Fever), salmonellosis, Sarcina lutea, Scabie, Schistomsomiasis, schistosomiasis, Serratia marcescens, Shigella, Shigella dysenteriae (Dysentery), Shigella flexneri-(Dysentery), Shigella paradysenteriae, Shigellosis, Smallpox, Spirillum rubrum, staphylococcal enterotoxin B, Staphylococcus Albus (Staph), Staphylococcus Aureus (Staph), Streptococcus hemolyticus, Streptococcus lactis, Streptococcus viridians, swine influenza, syphilis, Tinea, Tobacco mosaic, Trachoma, trichuriasis, trypanosomiasis, tuberculosis, Tularemia, Typhoid fever, typhus fever, ulcerans Calicivirus infection (Norovirus and Sapovirus), Umezaka, variola major, Venezuelan equine encephalitis, Venezuelan hemorrhagic fever, Vibrio cholera, viral encephalitis, Viral hemorrhagic fevers, yellow fever, Yersinia pestis, and Yersinia pestis.

### E-2. Diagnosis of Cancers

The devices and the methods in the present invention can be used for diagnosing cancers which include but not limit to:

Acute Lymphoblastic Leukemia (ALL), Acute Myeloid Leukemia (AML), Adolescents, Adrenocortical Carcinoma, Childhood Adrenocortical Carcinoma, AIDS-Related Cancers, Kaposi Sarcoma (Soft Tissue Sarcoma), AIDS-Related Lymphoma (Lymphoma), Primary CNS Lymphoma (Lymphoma), Anal Cancer, Gastrointestinal Carcinoid Tumors, Astrocytomas, Childhood (Brain Cancer), Atypical Teratoid/Rhabdoid Tumor, Childhood, Central Nervous System (Brain Cancer);

Basal Cell Carcinoma of the Skin—Skin Cancer, Bile Duct Cancer, Bladder Cancer, Childhood Bladder Cancer, Bone Cancer (includes Ewing Sarcoma and Osteosarcoma and Malignant Fibrous Histiocytoma), Brain Tumors, Breast Cancer, Childhood Breast Cancer, Bronchial Tumors, Childhood, Burkitt Lymphoma—Non-Hodgkin Lymphoma;

Carcinoid Tumor (Gastrointestinal), Childhood Carcinoid Tumors, Carcinoma of Unknown Primary, Childhood Carcinoma of Unknown Primary, Cardiac (Heart) Tumors, Central Nervous System tumors, Atypical Teratoid/Rhabdoid Tumor, Childhood (Brain Cancer), Embryonal Tumors, Childhood (Brain Cancer), Germ Cell Tumor, Childhood (Brain Cancer), Primary CNS Lymphoma, Cervical Cancer, Childhood Cervical Cancer, Childhood Cancers, Cancers of Childhood, Unusual, Cholangiocarcinoma—Bile Duct Cancer, Chordoma, Childhood—Unusual Cancers of Childhood, Chronic Lymphocytic Leukemia (CLL), Chronic Myelogenous Leukemia (CML), Chronic Myeloproliferative Neoplasms, Colorectal Cancer, Childhood Colorectal Cancer, Craniopharyngioma, Childhood (Brain Cancer), Cutaneous T-Cell Lymphoma—Lymphoma (Mycosis Fungoides and Sezary Syndrome);

Ductal Carcinoma In Situ (DCIS)—Breast Cancer,

Embryonal Tumors, Central Nervous System, Childhood (Brain Cancer), Endometrial Cancer (Uterine Cancer), Ependymoma, Childhood (Brain Cancer), Esophageal Cancer, Childhood Esophageal Cancer; Esthesioneuroblastoma (Head and Neck Cancer); Ewing Sarcoma (Bone Cancer); Extracranial Germ Cell Tumor, Childhood; Extragonadal Germ Cell Tumor; Eye Cancer; Childhood Intraocular Melanoma; Intraocular Melanoma; Retinoblastoma; Fallopian Tube Cancer; Fibrous Histiocytoma of Bone, Malignant, and Osteosarcoma; Gallbladder Cancer; Gastric (Stomach) Cancer; Childhood Gastric (Stomach) Cancer; Gastrointestinal Carcinoid Tumor; Gastrointestinal Stromal Tumors (GIST) (Soft Tissue Sarcoma); Childhood Gastrointestinal Stromal Tumors; Germ Cell Tumors; Childhood Central Nervous System Germ Cell Tumors (Brain Cancer); Childhood Extracranial Germ Cell Tumors; Extragonadal Germ Cell Tumors; Ovarian Germ Cell Tumors; Testicular Cancer; Gestational Trophoblastic Disease;

Hairy Cell Leukemia; Head and Neck Cancer; Heart Tumors, Childhood; Hepatocellular (Liver) Cancer; Histiocytosis, Langerhans Cell; Hodgkin Lymphoma; Hypopharyngeal Cancer (Head and Neck Cancer);

Intraocular Melanoma; Childhood Intraocular Melanoma; Islet Cell Tumors, Pancreatic Neuroendocrine Tumors; Kaposi Sarcoma (Soft Tissue Sarcoma); Kidney (Renal Cell) Cancer; Langerhans Cell Histiocytosis; Laryngeal Cancer (Head and Neck Cancer); Leukemia; Lip and Oral Cavity Cancer (Head and Neck Cancer); Liver Cancer; Lung Cancer (Non-Small Cell and Small Cell); Childhood Lung Cancer; Lymphoma;

Male Breast Cancer; Malignant Fibrous Histiocytoma of Bone and Osteosarcoma; Melanoma; Childhood Melanoma; Melanoma, Intraocular (Eye); Childhood Intraocular Melanoma; Merkel Cell Carcinoma (Skin Cancer); Mesothelioma, Malignant; Childhood Mesothelioma; Metastatic Cancer; Metastatic Squamous Neck Cancer with Occult Primary (Head and Neck Cancer); Midline Tract Carcinoma With NUT Gene Changes; Mouth Cancer (Head and Neck Cancer); Multiple Endocrine Neoplasia Syndromes; Multiple Myeloma/Plasma Cell Neoplasms; Mycosis Fungoides (Lymphoma); Myelodysplastic Syndromes, Myelodysplastic/Myeloproliferative Neoplasms; Myelogenous Leukemia, Chronic (CML); Myeloid Leukemia, Acute (AML); Myeloproliferative Neoplasms, Chronic;

Nasal Cavity and Paranasal Sinus Cancer (Head and Neck Cancer); Nasopharyngeal Cancer (Head and Neck Cancer); Neuroblastoma; Non-Hodgkin Lymphoma; Non-Small Cell Lung Cancer; Oral Cancer, Lip and Oral Cavity Cancer and Oropharyngeal Cancer (Head and Neck Cancer); Osteosarcoma and Malignant Fibrous Histiocytoma of Bone; Ovarian Cancer; Childhood Ovarian Cancer; Pancreatic Cancer; Childhood Pancreatic Cancer; Pancreatic Neuroendocrine Tumors (Islet Cell Tumors); Papillomatosis (Childhood Laryngeal); Paraganglioma; Childhood Paraganglioma; Paranasal Sinus and Nasal Cavity Cancer (Head and Neck Cancer); Parathyroid Cancer; Penile Cancer; Pharyngeal Cancer (Head and Neck Cancer); Pheochromocytoma; Childhood Pheochromocytoma; Pituitary Tumor; Plasma Cell Neoplasm/Multiple Myeloma; Pleuropulmonary Blastoma; Pregnancy and Breast Cancer; Primary Central Nervous System (CNS) Lymphoma; Primary Peritoneal Cancer; Prostate Cancer;

Rectal Cancer; Recurrent Cancer; Renal Cell (Kidney) Cancer; Retinoblastoma; Rhabdomyosarcoma, Childhood (Soft Tissue Sarcoma); Salivary Gland Cancer (Head and Neck Cancer); Sarcoma; Childhood Rhabdomyosarcoma (Soft Tissue Sarcoma); Childhood Vascular Tumors (Soft Tissue Sarcoma); Ewing Sarcoma (Bone Cancer); Kaposi Sarcoma (Soft Tissue Sarcoma); Osteosarcoma (Bone Cancer); Soft Tissue Sarcoma; Uterine Sarcoma; Sezary Syndrome (Lymphoma); Skin Cancer; Childhood Skin Cancer; Small Cell Lung Cancer; Small Intestine Cancer; Soft Tissue Sarcoma; Squamous Cell Carcinoma of the Skin; Squamous Neck Cancer with Occult Primary, Metastatic (Head and Neck Cancer); Stomach (Gastric) Cancer; Childhood Stomach (Gastric) Cancer;

T-Cell Lymphoma, Cutaneous—Lymphoma (Mycosis Fungoides and Sezary Syndrome); Testicular Cancer; Childhood Testicular Cancer; Throat Cancer (Head and Neck Cancer); Nasopharyngeal Cancer; Oropharyngeal Cancer; Hypopharyngeal Cancer; Thymoma and Thymic Carcinoma; Thyroid Cancer; Transitional Cell Cancer of the Renal Pelvis and Ureter (Kidney (Renal Cell) Cancer); Unknown Primary, Carcinoma of; Childhood Cancer of Unknown Primary; Unusual Cancers of Childhood; Ureter and Renal Pelvis, Transitional Cell Cancer (Kidney (Renal Cell) Cancer; Urethral Cancer; Uterine Cancer, Endometrial; Uterine Sarcoma; Vaginal Cancer; Childhood Vaginal Cancer; Vascular Tumors (Soft Tissue Sarcoma); Vulvar Cancer; Wilms Tumor and Other Childhood Kidney Tumors.

One aspect of the present disclosure is to provide devices and methods for easy and rapid tissue staining by utilizing a pair of plates that are movable to each other to manipulate a tissue sample and/or a small volume of staining liquid, reducing sample/staining liquid thickness, making a contact between the sample and staining reagent, etc.—all of them have beneficial effects on the tissue staining (simplify and speed up stain, wash free, and save reagent)

Another aspect of the present disclosure is to provide for easy and rapid tissue staining by coating staining reagents on one or both of the plate(s), which upon contacting the liquid sample and/or the staining liquid, are dissolved and diffuse in the sample and/or the staining liquid, easing the handling of staining reagents with no need of professional training.

Another aspect of the present disclosure is to ensure uniform access of the sample to the staining reagent by utilizing the plates and a plurality of spacers of a uniform height to force the sample and/or staining liquid to forma thin film of uniform thickness, leading to same diffusion distance for the staining reagents across a large lateral area over the sample.

Another aspect of the present disclosure is to provide systems for easy and rapid tissue staining and imaging by combining the pair of plates for staining with a mobile communication device adapted for acquiring and analyzing images of the tissue sample stained by the plates. Optionally, the mobile communication is configured to send the imaging data and/or analysis results to a remote location for storage and/or further analysis and interpretation by professional staff or software.

Another aspect of the present disclosure is to provide devices, systems and methods for immunohistochemistry.

Another aspect of the present disclosure is to provide devices, systems and methods for H&E stains, special stains, and/or cell viability stains.

Another aspect of the present disclosure is to provide devices, systems and methods for in situ hybridization.

Another aspect of the present disclosure is to provide devices, systems and methods for staining biological materials (e.g. for staining of cells or tissues, nucleic acid stains, H&E stains, special stains, and/or cell viability stains. etc.) without washing, and in some embodiments, in a single step.

### Using CROF Cards in Cytology/Cytopathology Screening and Diagnosis

Some embodiments of the present invention are related to collect and analyze a sample using cytology quickly and simply.

According to the present invention, a method of collecting and analyzing a sample using cytology comprising:
having a first plate and a second plate that are movable relative to each other;
collecting a biological sample (i.e. biopsy) from a subject (e.g. a human or animal);
depositing a part of the sample on an inner surface of a first plate;
depositing a staining solution on either (i) surface of the first plate and/or on top of the sample, (ii) inner surface of the second plate, or (iii) both,
bringing the two plate together to a closed configuration, in which, the two inner surfaces of the first and second plates are facing each other and the spacing between the plates is regulated by spacers between the plate, and at least a part of the staining solution is between the sample and the inner surface of the second plate;
having an imager; and
imaging the sample for analysis.

In some embodiments, the analysis by imaging is cyto-analysis.

In some embodiments, the spacers are fixed on one or both plates. In some embodiments, the spacers are inside of the staining solution.

In some embodiments, the sample is mixed with the staining solution before dropped on the plate.

In some embodiments, the staining solution comprises staining agent (things that stain cells/tissue) in a solution. In some embodiments, the staining solution does not comprises staining dye in a solution, but is configured to transport a staining agent coated on one of the plates into the cells/tissue. In some embodiments, the staining solution comprises staining agent (things that stain cells/tissue) in a solution, and is configured to transport a staining agent coated on one of the plates into the cells/tissue.

In some embodiments, the spacer height is configured to make the stained cells and/or tissues be visible by an imaging device without washing away the staining solution between the second plate and the sample.

In some embodiments, the spacer height is configured to make the stained cells and/or tissues be visible by an imaging device without open the plates after the plates reached a closed configuration.

In some embodiments, a sample was stained without washing away the staining solution between the second plate and the sample, and imaged by an imager, after closing the plates into a closed configuration, and without washing, imaged by an imager.

In some embodiments, a sample was stained without washing away the staining solution between the second plate and the sample, and imaged by an imager, after closing the plates into a closed configuration, in 30 seconds or less, 60 seconds or less, 120 seconds or less, 300 seconds or less, 600 seconds or less, or a range between any of the two.

In some preferred embodiments, a sample was stained without washing away the staining solution between the second plate and the sample, and imaged by an imager, after closing the plates into a closed configuration, in 30 seconds or less, 60 seconds or less, 120 seconds or less, or a range between any of the two.

In some preferred embodiments, a sample was stained without washing away the staining solution between the second plate and the sample, and imaged by an imager, after closing the plates into a closed configuration, in 30 seconds or less, 60 seconds or less, or a range between any of the two.

In some embodiments, the spacer height is 0.2 um (micron) or less, 0.5 um or less, 1 um or less, 3 um or less, 5 um or less, 10 um or less, 20 um or less, 30 um or less, 40 um or less, 50 um or less, or a range between any of the two.

In some preferred embodiments, the spacer height is 3 um or less. In some preferred embodiments, 10 um or less. In some preferred embodiments, 20 um or less. In some preferred embodiments, 30 um or less.

In some preferred embodiments, the staining solution has, after the plates are in a closed configuration, a thickness that is equal or less than sub-noise thickness.

The term “sub-noise thickness” (SNT) reference to the a thickness of a sample or a staining solution, which is thinner than a thickness where the noise in the sample or in the staining solution is below the signal from a specifically bound optical label, making the optical label visible to an imager. Making a staining solution less than the SNT will remove the need to wash away the unbind optical labels.

In some embodiments, the first plate and the second plate are connected by a hinge.

In some embodiments, the staining solution has a volume 2 uL(micro-liter) or less, 2 uL or less, 5 uL or less, 10 uL or less, 15 uL or less, 20 uL or less, 30 uL or less, 50 uL or less, 100 uL or less, or a range between any of the two.

In some preferred embodiments, the staining solution has a volume 2 uL(micro-liter) or less, 2 uL or less, 5 uL or less, 10 uL or less, 15 uL or less, 20 uL or less, 30 uL or less, or a range between any of the two.

In some preferred embodiments, the staining solution has a volume 2 uL(micro-liter) or less, 2 uL or less, 5 uL or less, 10 uL or less, 15 uL or less, or a range between any of the two.

Example of oral cancer diagnostics. According to the present invention, the sample is epithelial cells that exfoliated by a swab from the mouth of a subject. An oral cancer diagnostics can be done by measuring the size and/or area of an epithelial cell and its nucleus, and/or by measuring the ratio of the size and/or of them. For example, a cancer epithelial cell typically has a ratio of the areas (and/or size) of the nucleus to the area larger than that a norm epithelial cell.

Example of screen smoker from non-smoker. According to the present invention, the sample is epithelial cells that exfoliated by a swab from the mouth of a subject. An oral cancer diagnostics can be done by measuring the size and/or area of an epithelial cell and its nucleus, and/or by measuring the ratio of the size and/or of them. A smoker has an epithelial cell that typically has, compared with a non-smoker, a different ratio of the areas (and/or size) of the nucleus to the area larger than that a norm epithelial cell.

One application of the present invention is in cytopathology. Cytopathology is commonly used to investigate disease at cellular level using free cells or tissue fragments removed from a wide range of body sites. It has been the main tool utilized to screen and diagnose cancer and some infectious diseases or other inflammatory conditions. For example, a common application of cytopathology is the Pap smear, a screening tool used to detect precancerous cervical lesions that may lead to cervical cancer.

Some examples of the present invention in cytology/cytopathology are diagnosis based on haematoxylin and eosin (H&E) stained slides to the current regular evaluation of tumors by immunocytochemistry (ICC) and in situ hybridization (ISH) to confirm tumor histogenesis, subtype, and to provide additional information influencing prognosis and treatment in cancers.

In some embodiments, several ways are used to remove biopsy from a subject's body sites: needle, endoscopy and excisional or incisional surgery.

One aspect of the present invention is the devices and methods for performing a biopsy processing and staining in simple way. Different from standard cytopathology of preparing sample, sampling on slide, followed by staining biopsy on slide, exemplary embodiments advantage the three steps into one single step, in detail, place drop, slice, or block of biopsy material directly onto underneath card holder which can be conventional glass slide, a micrometer height chamber, or any type of holder. Place X-plate pillar side down and gently press X-plate together with staining solution onto biopsy samples. Pressing X-plate onto sample is able to spread bulk of sample into monolayer which thickness prefixed by pillar height (10 um). with two main features: 1) predefined micrometer (5 um, 10 um or higher) height pillar on X-plate for evenly processinq/spreadinq biopsy samples into single cell based monolayer without changing cell's or tissue's morphology; 2) predefined microvolume chamber significantly fast staining time to less than 1 min.

Using microvolume embodiment for cytopathology 1) sample processing and 2) staining.

For example, in certain embodiments, the QMAX device is used to process (press) biopsy material to monolayer. Biopsy material can be collected from:

Sample Taking Methods. In some embodiments, a biopsy sample is removed by using one or a combination of the following methods: needle aspiration, endoscopy and excisional or incisional surgery.
needle biopsy from skin lesion, lymph node, thyroid, mammary gland, lung and body cavity
tissue Smear from oral brush material, cervical (pap smear), body fluid: urine, sputum (phlegm), spinal fluid, pleural fluid, pericardial fluid, ascitic fluid.
endoscopy biopsy from
GI tract: esophagus, stomach, and duodenum (esophagogastroduodenoscopy), small intestine (enteroscopy), large intestine/colon (colonoscopy, sigmoidoscopy), bile duct, rectum (rectoscopy), and anus (anoscopy);
respiratory tract: nose (rhinoscopy), lower respiratory tract (fiberoptic bronchoscopy)
Ear: otoscopy
urinary tract: cystoscopy
female reproductive tract (gynoscopy): cervix (colposcopy), uterus (hysteroscopy), fallopian tubes (falloposcopy).
through a small incision: abdominal or pelvic cavity (laparoscopy), interior of a joint (arthroscopy), organs of the chest (thoracoscopy and mediastinoscopy).
Surgery biopsy from any excisionally or incisionally removed tissue or mass

In certain embodiments, the QMAX device is used to stain any molecular, organelle, cellular, outer cellular or organoid structure.
biological molecule include, but not limited to: protein, peptide, amino acids (selenocysteine, pyrrolysine, carnitine, ornithine, GABA and taurine), lipid (glycolipids, phospholipids, sterols, arachidonic acid, prostaglandins, leukotrienes), fatty acids, carbohydrates (monosaccharides, disaccharides, polysaccharides), nucleic acids (nucleotide, oligonucleotide, polynucleotides), any catabolites, any metabolites, secondary metabolites, vitamins, reactive oxygen/nitrogen species, minerals, polyphenolic macromolecule, and other small molecules, etc.
modification/reaction of biological molecules include, but not limited to: phosphorylation, methylation, acetylation, lipidation, thiol reactions, amine reaction, carboxylate reactions, hydroxyl reactions, aldehyde and ketone reactions, etc.
cellular organelle/subcellular structure include, but not limited to: nucleus, ribosome, peroxisomes, endoplasmic reticulum, golgi apparatus, mitochondria, lysosome, cell membrane, endosome, exosome, cytoskeleton.
type of cells with any physiological/pathological conditions include, but not limited to: within a tumor (can be originated from any epithelial from any organ, and vessel endothelial cells, fibroblast, lymphocyte), neuronal cells, lipocytes, stromal cells, chondrocytes, retinal cells, glial cells, smooth muscle cells, any type of stem cells, any type of embryonic cells, any type of endocrine cells, any type of exocrine cells, any type of immune cells, dendritic cells, myeloid cells, hematopoietic cells, lymphocyte, normal cells, benign cells, premalignant cells, malignant cells, transformation cells, quiescent cells, proliferation cells, apoptotic cells, senescent cells, mitotic cells, inflammatory cells, hyperplasia cells, hypertrophy cells, atrophy cells, hyperplasia cells, dysplasia cells, metaplasia cells etc.
connective tissue lextracellular structures include, but not limited to: Loose ordinary connective tissue, adipose tissue, blood and blood forming tissues, dense ordinary connective tissue, cartilage, bone, any type of extracellular vesicles, extracellular matrix, platelet, etc.

In some embodiments, the materials and methods of staining include:
a. Dye staining
   Papanicolaou staining: Harris hematoxylin; orange G6; EA50 (eosin Y, light green SF)
   May-Grunwald Giemsa staining (eosin G, methylene blue)
   Ziehl-Neelsen stain
   Modified Ziehl Neelson (for acid fast bacilli), Gram staining (Bacteria), Mucicarmine (mucins), PAS (for glycogen, fungal wall, lipofuscin, etc), Oil red O (lipids), Perl's Prussian blue (iron), modified Fouchet's test (bilirubin),
   any fluorescent/non-fluorescent dye for biological molecule, organelles, cells and biological structures, for example nuclei acid dyes: cyanine dyes (PicoGreen, OliGreen and RiboGreen, SYBR Gold, SYBR Green I and SYBR Green 1l, CyQUANT GR dye), cyanine dimer dyes (SYTOX, POPO-1, TOTO-1, YOYO-1, BOBO-1, JOJO-1, POPO-3, LOLO-1, TOTO-3, PO-PRO-1, JO-PRO-1, YO-PRO-1, PO-PRO-3, YO-PRO-3, TO-PRO-3, TO-PRO-5), amine-reactive cyanine dye (SYBR 101 dye), phenanthridines and acridines (ethidium brornide (EB) and ethidium homodimer-1, propidium iodide (PI), acridine orange (AO), hexidium iodide, dihydroethidium, ethidium homodimer-1, ethidium homodimer-2, ethidium monoazide, acridine homodimer bis-(6-chloro-2-methoxy-9-acridinyl)sperrnine, ACMA), Indoles and Imidazoles (Hoechst 33258, Hoechst 33342, Hoechst 34580, DAPI), 7-Aminoactinomycin D and Actinomycin D, Hydroxystilbamidine, LDS 751, Nissl Stains
b. IHC/IF staining
   Direct method, indirect method, PAP method (peroxidase anti-peroxidase method), Avidin-Biotin Complex (ABC) Method, Labeled StreptAvidin Biotin (LSAB) Method, Polymeric Methods (EnVision Systems based on dextran polymer technology, ImmPRESS polymerized reporter enzyme staining system), CAS system (from DAKO), CSA II—Biotin-free Tyramide Signal Amplification System
c. ISH/FISH
   Method: direct and indirect methods
   Probes: double-stranded DNA (dsDNA) probes, single-stranded DNA (ssDNA) probes, RNA probes (riboprobes), synthetic oligonucleotides labelling probes: for example, DIG (digoxigenin), biotin, fluorophore (FITC, alexa, tyramide, etc.)
d. Other Materials

Acridine orange (50 ug/ml) and hematoxylin staining solution (from Vector Laboratories) were used in this study.

Sample holders. The sample holder comprises X-plate with spacers/pillars that have a substantially uniform height and a nearly uniform cross-section separated from one another by a consistent, defined distance.

For examples X-Plate is 175 um thick PMMA with a pillar array of 30×40 um pillar size, 10 um pillar height and 80 um inter space distance, or iMOST Q-Card comprising X-plate with 175 um thick PMMA with a pillar array of 40 um diameter pillar size, 10 um pillar height and 120 um inter space distance.

### Example A

INSH (intra cellar detection inside cell using nucleic acid as probe) Sars-cov-2 specific mRNA probes staining of nasopharyngeal epithelial cells to diagnose COVID-19

The genome of human SARS-CoV-2 shares 88% similarity with human SARS-CoV-1, 29% with human MERS, and 99% with Bat coronavirus RaTG13 as shown in FIG. 1. To specific diagnose COVID-19 using iMOST, I designed 11 listed probes targeting Orf1ab, Orf3 and Spike mRNAs of SARS-COV-2.

Orf1ab 5289-5349

tagagcaggtggattaaacttcaactctatttgttggagtgttaacaatgcagtggcaag

Orf1ab 6262-6322

caactggttttgtgctccaaagacaacgtatacaccaggtatttggtttatacgtgg ctt

Orf1ab 6702-6762

agtacaaacacggtttaaacaccgtgtaactatgttagtagttgtactaacaactttgtt

Orf1ab 20344-20404

Ggtgattccttaaaacgtttagctagtccaatcagtagatgtaaaccacctaactgacta

Orf1ab 1550-1607

Ttgtcattaagaccttcggaaccttctccaacaacacctgtatggttacaacctatgtta

Orf3a 25687-25746

Ttatactctgcaagaagtagactaaagcataaagatagagaaaaggggcttcaaggccag

Orf3a 25409-25469

Tgaaggagtagcatccttgatttcaccttg cttcaaagttacagttccaattgtgaagat

Spike 21750-21756

Cctcttagtaccattggtcccagagacatgtatagcatggaaccaa

Spike 21995-22053

Ttcgcactagaataaactctgaactcactttccatccaacttttgttgtttttgtggta

Spike 22276-22336

Ccaacctgaagaagaatcaccaggagtcaaataacttctatgtaaagcaagtaaagtttg

Spike 22291-22349

cagcaccagctgtccaacctgaagaagaatcaccaggagtcaaataacttctatgtaaa

Sampling and Staining:

Mix nasopharyngeal cytology swab with 100 ul of saline in a clean eppendorf tube. Add 3-10 ul of nasopharyngeal saline mix onto the bottom plate of a Q-Card with dry print/coat Sars-cov-2 specific mRNA probes on the top plate of the Q-Card (X-plate); Close the Q-Card and incubate the specimen sample with the staining solution at room temperature for less than 1 min; and Image, record, and analyze, the nasopharyngeal epithelial cells in the closed Q-Card using an Phone having an adapter or a fluorescent microscope.

Experimental Results: Observation of Sars-cov-2 mRNA (+) epithelial cells can be diagnosed as COVID-19.

### Example B

### ISIM (Intra Cellar Detection Inside Cell Using Protein as Probe) Sars-Cov-2 Specific Antibody Staining of Nasopharyngeal Epithelial Cells to Diagnose COVID-19 Sampling and Staining:

Mix nasopharyngeal cytology swab with staining solution including PBS, Zwittgent 3-14, ethanol, and AF488-anti-Spike or AF488-anti-Nucleocapsid protein antibody;

Close the Q-Card and incubate the specimen sample with the staining solution at room temperature for less than 1 min; and

Image, record, and analyze, the nasopharyngeal epithelial cells in the closed Q-Card using an Phone having an adapter or a fluorescent microscope.

Experimental Results: Observation of AF488-anti-Spkie (+) or AF488-anti-Nucelocapsid protein (+) epithelial cells can be diagnosed as COVID-19.

### Examples of INSA Applicable Diseases:

Swab samples: coronavirus, including Sars-cov-1, Mers and Sars-cov-2 (COVID-19)

### Diagnostic Markers:

| Disease | source | biomarker |
|---|---|---|
| Coronavirus (Sars, Mers, | Nasal swab | spike, nucleocapsid, orf1a, |
| COVID-19) | | orf1ab, orf3a, orf6, orf7a, |
| | | orf7b, orf10, membrane |
| | | glycoprotein, envelop protein |
| | Saliva | spike, nucleocapsid, orf1a, |
| | | orf1ab, orf3a, orf6, orf7a, |
| | | orf7b, orf10, membrane |
| | | glycoprotein, envelop protein |
| | Blood | spike, nucleocapsid, orf1a, |
| | | orf1ab, orf3a, orf6, orf7a, |
| | | orf7b, orf10, membrane |
| | | glycoprotein, envelop protein |

In some embodiments, A method of rapidly detecting an analyte inside a sample by sandwiching the sample between two plates, binding the analyte inside a cell with a probe, and imaging, wherein the distance spacing between the two plates and the probe concentration are optimized for detection of the analyte in the cell without washing, comprising:
(a) obtaining a sample that contains at least a cell, wherein the sample contains or is suspected of containing an analyte, wherein the concentration of the analyte inside the cell is higher than that outside the cell;
(b) obtaining a probe that binds the analyte, wherein the probe has a configured probe concentration;
(c) obtaining a sample holder comprising two plates that are arranged or can be arranged to face each other with a configured plate-spacing between them;
(d) sandwiching the sample and the probe between the two plates to form a sample-probe thin layer, wherein the sample-probe thin layer comprises at least a volume A and at least a volume B, wherein at least a part of the sample-probe thin layer is regulated by the plate-spacing between two plates, and comprise a volume A and B, and wherein volume A contains at least a fraction of the cell, and volume B does not contain the cell;
(e) imaging, after step (d) and without washing away the unbound probe and the two plates are arranged at the configured spacing, a signal from at least the sample-probe thin layer to produce one or more images, wherein the one or more images comprises a signal from the probe in volumes A and B; and
(f) comparing the image of volume A to the image of volume B to determine whether the sample contains the analyte;
wherein the configured probe concentration and the configured plate-spacing are configured to make, in imaging step (e), the image of volume A distinguishable from the image of volume B in the imaging of the step (e), and
wherein the plate-spacing in imaged in step (e) is 250 um or less.

A variety of applications for the method, including several diagnostic applications, are described below.

In the present method, an intracellular analyte may be rapidly detected while the sample is sandwiched between two plates. The method may be done in the absence of a washing step (i.e., in the absence of a step in which unbound probe is washed from the cells), where both the spacing between the plates in the sandwich and concentration of the probe are optimized for detection of the analyte without washing. In other words, the probe concentration and the plate-spacing are optimized such that an image of volume A (which contains a fraction, i.e., a portion, of a cell that contains the analyte) is distinguishable from the image of volume B (which does not contain a cell).

In any embodiment, the cell should be permeabilized, e.g., using a detergent or other permeabilization agent. The degree of permeabilization should be sufficient to allow equilibration of probe binding to the biomarker, in should be insufficient to provide leakage of the contents of the cell into the surrounding milieu.

In any embodiment, the type of cell is not analyzed or determined in the method. In some embodiments, however, the outline of the cell in the image may be determined.

Diagnostic Methods

The FICA method may be employed in several diagnostic methods, e.g., to detect cells related a disease and/or disorder. In these embodiments, the method may comprise: (a) obtaining a sample that is suspected of containing or that contains a cell that is affected by a disease or disorder (e.g., a cell that has been infected by an infectious agent or a cancerous cell); (b) depositing the sample on a QMAX card and closing the QMAX into a closed configuration; (c) allowing a labeled probe get inside the cell, wherein the labeled probe specifically binds a biomarker that is specific to the disease or disorder; and (d) detecting the labeled probe inside the cell, without washing (i.e., without washing away the probe molecules that have not bound to the biomarker). The methods may employ cytology, immuno-histochemistry or in situ hybridization, or any combination thereof.

In some embodiments, the cell may be stained by a probe that is contained in a staining reagent. The staining reagent can be added to the sample in liquid, e.g., before or after the sample has been deposited onto a plate. Alternatively the staining reagent can be dried onto one or both of the plates such the probe dissolves into the sample after the sample has been deposited onto a plate. In any embodiment, a staining reagent may have a pH in the range of 7-8, and may be suitable for antibody staining. For example, the staining reagent, in liquid form, may be phosphate-buffered saline (PBS), saline, Tris buffer, HEPES buffer, or sodium bicarbonate, for example.

In any embodiment, a cell may be permeabilized in order to allow a staining reagent, e.g., a probe, into the cell. In these embodiments, the cells are permeabilized in situ, i.e., while they are in the device. Permeabilization of the cells can be done by a variety of different methods including, but not limited to: treatment with a Biomarkers for detecting disease are numerous and well known. In some embodiments, the biomarker bound by the probe may be a nucleic acid (e.g., an RNA, such as an mRNA or miRNA, or a DNA). In other embodiments, the biomarker is a protein. In any embodiment, the biomarker can be intracellular, i.e., present within a cell, e.g., in the nucleus or cytoplasm, for example. The biomarker may be, but not limited to: protein, peptide, amino acids (selenocysteine, pyrrolysine, carnitine, ornithine, GABA and taurine). lipid (glycolipids, phospholipids, sterols, arachidonic acid, prostaglandins, leukotrienes), fatty acids, carbohydrates (monosaccharides, disaccharides, polysaccharides), nucleic acids (nucleotide, oligonucleotide, polynucleotides), any catabolites, any metabolites, secondary metabolites, vitamins, reactive oxygen/nitrogen species, minerals, polyphenolic macromolecule, and other small molecules. A biomarker may be have been modified by, e.g., phosphorylation, methylation, acetylation, lipidation, thiol reactions, amine reaction, carboxylate reactions, hydroxyl reactions, aldehyde and ketone reactions. The biomarker may exist in a cellular organelle/subcellular structure, e.g. the nucleus, ribosome, peroxisomes, endoplasmic reticulum, golgi apparatus, mitochondria, lysosome, cell membrane, endosome, exosome, or cytoskeleton.

The sample analyzed using the present method contains cells. In some embodiments, the sample may be obtained from a tissue biopsy. For example, in some embodiments, the biopsy may be a fine needle aspirate, where a fine needle aspirate uses a thin, hollow needle to draw cells from a tissue (most commonly from the breast, thyroid gland or lymph nodes in the neck, groin, or armpit). In other embodiments, the biopsy analyzed by the present device may be a fluid taken from a bodily cavity, i.e., a “bodily fluid”, including, but not limited to, urine, sputum (phlegm), spinal fluid (from the space surrounding the brain and spinal cord), pleural fluid (from the space around the lungs), pericardial fluid (from the sac that surrounds the heart) or ascitic fluid (from the space in the belly). Alternatively, the biopsy may contain an exfoliate, which can be obtained using a scrape or brush. In exfoliative methods, a small spatula and/or brush may used to remove cells from a tissue for a test. For example, cells may be removed from the cervix (the lower part of the uterus or womb) for a Pap test. Other areas that can be brushed or scraped include the esophagus (swallowing tube), stomach, bronchi, mouth and skin. Cells can also be swabbed from a tissue (e.g., from the nose). In these embodiments, the sample may be deposited, e.g., smeared, directly onto the device. In other embodiments, the sample analyzed by the present device may be a section of a tissue biopsy. In these embodiments, a sample of tissue may be obtained, e.g., using a core needle (a hollow tube that allows the doctor to extract a core of tissue for testing) that is sectioned, e.g., by hand, prior to deposition onto the device. This “imprecise” sectioning can be done on sections that not been frozen or fixed In any embodiment, the cells deposited onto the device may be “fresh” in that they are not fixed or lysed at the time that they are deposited onto the device. Samples that contain free cells, e.g., blood cells or bone marrow cells, can also be analyzed.

In exemplary embodiments, the cell may be an epithelial cell (which are typically collected using a brush or scrape, or in fluids collected from a bodily cavity), although any other type of cell may be used. If the sample contains an epithelial cell, the sample may be removed from the nose, throat, mouth, the cervix, esophagus, stomach, bronchi, skin, or any other type of cell that lines a surface of an organ or tissue in a body, using a scrape, brush or swab, for example. In some embodiments, the cell may be from within a tumor (can be originated from any epithelial from any organ, and vessel endothelial cells, fibroblast, lymphocyte), neuronal cells, lipocytes, stromal cells, chondrocytes, retinal cells, glial cells, smooth muscle cells, any type of stem cells, any type of embryonic cells, any type of endocrine cells, any type of exocrine cells, any type of immune cells, dendritic cells, myeloid cells, hematopoietic cells, lymphocyte . . . : normal cells, benign cells, premalignant cells, malignant cells, transformation cells, quiescent cells, proliferation cells, apoptotic cells, senescent cells, mitotic cells, inflammatory cells, hyperplasia cells, hypertrophy cells, atrophy cells, hyperplasia cells, dysplasia cells, metaplasia cells, . . .

For example, in some embodiments, a biopsy sample is removed by using one or a combination of the following methods: needle aspiration, endoscopy and excisional or incisional surgery. A needle biopsy may from skin lesion, lymph node, thyroid, mammary gland, lung and body cavity; a tissue smear may be an oral brush material, cervical (pap smear), body fluid: urine, sputum (phlegm), spinal fluid, pleural fluid, pericardial fluid, ascitic fluid; an endoscopy biopsy may be from GI tract: esophagus, stomach, and duodenum (esophagogastroduodenoscopy), small intestine (enteroscopy), large intestine/colon (colonoscopy, sigmoidoscopy), bile duct, rectum (rectoscopy), and anus (anoscopy); respiratory tract: nose (rhinoscopy), lower respiratory tract (fiberoptic bronchoscopy); ear: otoscopy; urinary tract: cystoscopy; female reproductive tract (gynoscopy): cervix (colposcopy), uterus (hysteroscopy), fallopian tubes (falloposcopy). or through a small incision: abdominal or pelvic cavity (laparoscopy), interior of a joint (arthroscopy), organs of the chest (thoracoscopy and mediastinoscopy), for example. A surgery biopsy from any excisionally or incisionally removed tissue or mass. In certain embodiments, the QMAX device is used to stain any molecular, organelle, cellular, outer cellular or organoid structure, for example.

In any embodiment, the staining reagent (which reagent may include a labeled antibody, aptamer or nucleic acid probe, for example), and, optionally, a cell permeabilization agent, may be in dried form and coated onto one or more of the plates in the QMAX card. In these embodiments, the staining reagent and cell permeabilization agent may dissolve in the sample after they are contacted therewith, thereby permeabilizing and staining the cells, as needed. In other embodiments, the staining reagent (which reagent may include a labeled antibody or nucleic acid probe, for example), and, optionally, a cell permeabilization agent, may be added to the sample in liquid for, either before or after the sample has been deposited onto the QMAX plate.

In any embodiment, the staining can be rapid, meaning that a signal from sandwiched layer can be read within seconds (e.g., less than a minute) or minutes (e.g., less than 5 minutes). In these embodiments, the spacing between the two plates is configured to make the saturation time for the binding between the analyte and the probe is 10 seconds or less, 20 seconds or less, 30 seconds or less, 60 seconds or less, 90 seconds or less, 120 seconds or less, 240 seconds or less, 300 seconds or less, 500 seconds or less, or a range between any of the two. In a preferred embodiment, the spacing between the two plates is configured to make the saturation time for the binding between the analyte and the probe is 10 seconds or less, 20 seconds or less, 30 seconds or less, 60 seconds or less, 90 seconds or less, or 120 seconds or less. In a preferred embodiment, the spacing between the two plates is configured to make the saturation time for the binding between the analyte and the probe is 10 seconds or less, 20 seconds or less, 30 seconds or less, or 60 seconds or less. In some embodiments, the sample can be imaged after binding is saturated.

As noted above, both the spacing between the plates in the sandwich and concentration of the probe are optimized for detection of the analyte without washing. In any embodiment, wherein the spacers on the QMAX card have a height of 0.5 um, 1 um, 2 um, 3 um, 5 um, 10 um, 15 um, 20 um, 30 um, 50 um, or a range between any two of the values.

In some preferred embodiments, the spacers on the QMAX card have a height of 0.5 um, 1 um, 2 um, 3 um, 5 um, 10 um, 15 um, or a range between any two of the values. The concentration of the probe used in the method can be empirically determined given the identify of the probe (e.g., whether it is an antibody, a nucleic acid, or a stain, etc.) and spacing between the plates.

Infectious Diseases

The present method can be used to diagnose any number of infections and diseases, e.g., bacterial infections, viral infections, fungal infections, infections by another type of infectious agent, or a

Histochemical Staining

In some embodiments, the method may comprise performing a histological assessment of cell morphology, where the term “histological assessment” in cancer diagnostics seeks to identify malignant cells in the sample based on the morphologic characteristics of the cells and cellular features.

In some embodiments, in visually assessing a tissue section on a slide, the tissue may be stained to assist in visualizing morphological changes to cells and cellular components (e.g. nuclei). Common stains used for histochemical include Haematoxylin and Eosin, Papanicolaou (Pap) and Alcian Blue Periodic Acid-Schiff stains. “Special stains” can often be used to identify different cell types. For example, a special stain can be used to identify the likely source of a metastasized cell.

In any embodiment, a benign tumor may appear as a homogenous proliferation of normal cells, whereas a cancer (i.e., malignant tumor) may appear as a group of dissimilar cells with a bigger nucleus (which can identified by hematoxylin staining) and some with mitotic figures (i.e., tangled, dark-staining threads) instead of a nucleus.

Haematoxylin and Eosin Staining: Hematoxylin reacts with cells like a basic dye and has a purplish blue colour. This stein dyes acidic, or basophilic, structure including the cell nucleus (which contains DNA and nucleoprotein), and organelles that contain RNA, e.g. ribosomes and the rough endoplasmic reticulum. Eosin is an acidic dye that is typically reddish or pink. It stains basic or acidophilic structures, including the cytoplasm, cell walls, and extracellular fibres,

Alcian Blue and PAS: This stain is a combined method utilising the properties of both the PAS and Alcian blue methods to stain all of the tissue proteoglycans. The acidic mucins are first stained with Alcian blue and those remaining acidic mucins (which are also PAS positive) will be chemically blocked. Those neutral mucins (which are solely PAS positive) will subsequently be demonstrated in a contrasting manner. Where mixtures occur, the resultant colour will depend upon the dominant moiety.

Papanicolaou (Pap), ultrafast Papanicoiaou UFP and Romanowsky: This is also referred to as “pap-staining” or a “pap smear”. This technique is commonly used to examine cell samples that have been obtained from body fluids. The technique can involve the combination of chemicals that include, for example, light green SF yellowish, haematoxylin, orange G and eosin Y (although there are many variations). Papanicolaou (PAP) staining was first described by Papanicolaou in 1943 and widely used as a screening test (see, e.g., Izar et al J Cytol. 2014; 31:154-7). The original PAP stain has been modified several times (see, e.g., Gill G W. Enviro-Pap: An environmentally friendly, economical, and effective Pap stain. Lab Med Indianapolis. 2006; 37:105-8). Ultrafast Papanicolaou (UFP) stain was introduced by Yang and Alvarez in 1995 (see, e.g., Choudary et al J Cytol. 2012; 29:241-5). UFP stain is a hybrid of air dried Romanowsky preparation and wet fixed PAP preparation. This method incorporates principles of air drying of cells, followed by rehydration in normal saline and fixation in alcoholic formalin. Air drying makes the cells appear larger and thus increase the resolution for analyzing cellular details.

Gram staining: Gram staining is a common staining technique, typically used to differentiate bacteria species as either gram positive or gram negative. This is achieved through the chemical properties of bacterial cell walls, where different colors are displayed after staining.

A variety of special stains can also be used:

Masson's Trichrome (skin): This stain is intended for analysis of collagenous connective tissue fibers in tissue specimens. This stain can be used to differentiate collagen and smooth muscle in tumors and assists in the detection of diseases or changes in connective/muscle tissue.

Modified GMS Silver Stain: The Modified GMS Silver stain can be used to analyze fungi, basement membrane and some opportunistic organisms such as Pneumocystis carinii in tissue specimens. This stain can be used to detect a fungal infections (e.g., an Aspergillus infection of the lung).

Periodic Acid Schiff: PAS staining can be used to identify structures containing a high proportion of carbohydrates such as glycogen, glycoproteins, proteoglycans typically found in connective tissues, mucus and basement membranes. This stain is commonly used to investigate kidney biopsies, liver biopsies, certain glycogen storage diseases in striated muscles and suspected fungal infections.

Pers' Prussian Blue Iron: This stain can be used to detect and identify ferric (Fe³⁺) iron in tissue preparations, blood smears, or bone marrow smears, for example. Small amounts of ferric iron (found in haemosiderin) are commonly found in bone marrow and in the spleen. Abnormal amounts of iron can indicate hemochromatosis and hemosiderosis.

Ziehl Neelsen: This stain is used to identify acid fast bacilli in tissue. Bacilli are rod-shaped bacterial organisms. For example, this stain is to identify tuberculosis in lung tissue.

Alcian Blue: This stain identifies acid mucopolysaccharides and acidic mucins. Excessive amounts of non-sulfated acidic mucosubstances are seen in mesotheliomas, although lesser amounts occur normally in blood vessel walls but increase in early lesions of atherosclerosis.

Gomori Trichrome: This stain can be used to stain and identify muscle fibers, collagen and nuclei. They can be used to contrast skeletal, cardiac or smooth muscle. The Gomori Trichrome is a simplification of the more elaborate Masson trichrome stain (described above) and combines the plasma stain (chromotrope 2R) and connective tissue stain to provide a brilliant contrasting picture.

As well as providing a way to analyze the morphology of a cell, a cell stain can be used to detect infections and infectious agents. For example, certain stains can be used to detect viral infections (e.g., HSV, VZV, CMV, adenovirus, HPV, etc), Chlamydia trachomatis and rickettsiae (using Giemsa), bacterial, mycobacterial (e.g., Mycobacterium tuberculosis), fungal (e.g., Actinomyces or Aspergillus) and parasitic infections. Examples of several stains that can be used to detect infections and infectious agents are described in great detail in Woods et al (Clin. Micro. Rev. 1996 9: 382-404), which is incorporated by reference herein.

Immunohistochemical Stains for Cancer

In many cases, the first step of many analysis methods is to identify the lineage of a neoplasm, i.e., whether it is epithelial, mesenchymal, or hematopoietic. Once the initial classification is made, additional stains can be used to further identify the source neoplasm (see, e.g., Painter et al, Toxicol. Pathol. 2010 38: 131-141 and Bahrami et al Arch Pathol Lab Med. 2008 132:326-48)). As would be recognized all of the markers described below can be identified using labeled antibodies.

Lineage Markers

Cytokeratins are found in epithelial cells of all types and can be used as a marker for that lineage. These proteins can be classified into several subtypes, numbered 1 through 20, and their expression is frequently organ-specific and/or tissue-specific. In addition to the type of epithelium, the subtypes of cytokeratins that epithelial cells express depend on the stage in the sequence of terminal differentiation and the stage of development. The low-molecular-weight cytokeratins (LMW CKs; including CK8, CK18, and CK19), recognized by the antibodies CAM 5.2 or 35BH11, and a cocktail of keratins (pankeratin), recognized by the antibody AE1/AE3, can be used to identify epithelia cells. Once the initial differentiation is made, the cells may be stained for specific cytokeratins (such as CK7 and CK20), which can can be used to better characterize an epithelial tumor. Epithelial Membrane Antigen (EMA) can be used as a supplement cytokeratins to identify cells that have differentiated from the epithelium, particularly in sarcomatoid carcinoma or those undifferentiated carcinomas that are negative for cytokeratins.

Vimentin is present in most mesenchymal cells and can be used as a marker for that lineage. Vimentin is present in almost all sarcomas and melanomas but is variable in lymphomas and even some carcinomas. Vimentin may be co-expressed with cytokeratin in a wide range of carcinomas and other tumors. Neoplasms that co-express cytokeratin and vimentin include renal, endometrial, thyroid, colonic, small intestinal, prostate, transitional cell and some ovarian carcinomas, synovial cell sarcoma, mesothelioma and carcinosarcoma.

CD45 can be used to as a marker for the hematopoietic lineage and, as such, can be used to identify hematopoietic neoplasms, e.g., those of the lymphoid lineage. Once this lineage has been determined, additional markers such as CD3 (a marker for T-cells), CD79a (a B-cell marker), and/or PAX-5 (another B-cell marker) can be used to better distinguish lymphoid cell types. Subsets of neoplastic T-cells can be further characterized using CD4 or CD8 markers. Lysozyme, vimentin, CD68, ED1/ED2 or F4/80 can be used as a diagnostic for histiocytic sarcoma, a systemic neoplasm of histiocytes/macrophages. Peripheral Nerve Sheath Tumors (PNST) (e.g., Schwannoma, Neurofibroma) can be detected by staining or S100, Leu-7, and Protein Gene Product 9.5. Likewise, muscle tumors can identified using vimentin & desmin, smooth muscle actin can identify smooth muscle tumors, myogenin can be used to detect skeletal muscle tumors and neuroendocrine tumors can be detected using synaptophysin or chromogranin.

S100 protein or HMB-45 can be used to identify malignant melanoma. S100 protein is a highly sensitive way to identify primary and metastatic sites of this cancer. A positive S100 requires both nuclear and cytoplasmic staining. To confirm that a S100-positive neoplasm is melanocytic, the tumor may be also positive for a melanocyte-specific protein (e.g., HMB-45 or MART-1/Melan-A). HMB-45 is another highly sensitive marker for melanoma, labeling almost 100% of conventional primary melanomas.

The following table summarizes the markers that are associated with various “small round cell tumors”, term is used to describe neoplasms composed of relatively small, round to oval, closely packed undifferentiated cells with high nuclear-cytoplasmic ratio, scant cytoplasm, and round nuclei with evenly distributed, slightly coarse chromatin and small or inconspicuous nucleoli, small round cell tumors include pathologic entities from very different images and include (1) epithelial tumors, for example, small cell carcinoma (SmC) (poorly differentiated neuroendocrine carcinoma); (2) mesenchymal tumors encompassing malignant solid neoplasms of childhood and other small round cell sarcomas; and (3) tumors with overlapping features, such as lymphoma and melanoma. Because of similar routine light microscopic features of these tumors, immunohistochemistry is often mandated for a definitive diagnosis.

| | | | | | | | | Myogenin/ | | |
|---|---|---|---|---|---|---|---|---|---|---|
| | S100 | CD99 | CK | CD45 | S100 | CD99 | Desmin | Myo-D1 | CD56 | WT1 |
| Poorly | − | + | + | − | − | + | − | − | + | − |
| differentiated | | | | | | | | | | |
| synovial sarcoma | | | | | | | | | | |
| Mesenchymal | − | + | − | − | − | + | ~ | − | ~ | − |
| chondrosarcoma | | | | | | | | | | |
| Wilms tumor | − | − | ~ | − | − | − | + | − | + | + |
| Ewing | − | + | ~ | − | − | + | − | − | − | − |
| sarcoma/primitive | | | | | | | | | | |
| neuroectodermal | | | | | | | | | | |
| tumor | | | | | | | | | | |
| Rhabdomyosarcoma | − | − | − | − | − | − | + | + | + | − |
| Desmoplastic | − | ~ | + | − | − | ~ | + | − | ~ | + |
| small round cell | | | | | | | | | | |
| tumor | | | | | | | | | | |
| Neuroblastoma | − | − | − | − | − | − | − | − | + | − |
| Small cell | − | − | + | − | − | − | − | − | + | − |
| carcinoma | | | | | | | | | | |
| Hematopoietic | − | − | − | + | − | − | − | − | − | − |
| malignancies | | | | | | | | | | |
| Melanoma | + | ~ | − | − | + | ~ | − | − | − | − |

Tissue-Specific and Organ-Specific Markers

Once the source of a carcinoma by the analysis of lineage markers has been established, immunohistochemistry may assist further by delineation of the cell line of differentiation. Further delineation of the neaoplasm can be achieved by analysis of cytokeratin subtypes, supplemented by other complementary or tissue-specific or organ-specific markers. Some markers are expressed almost exclusively in a specific tissue (e.g., PSA and PAP for prostate and uroplakin Ill for urothelial epithelium), whereas others are not strictly organ-specific or tissue-specific. The table below lists sever markers and their staining pattern in cancerous cells.

| Marker | Tumor | Staining Pattern |
|---|---|---|
| TTF-1 | Lung, thyroid | Nuclear |
| Thyroglobulin | Thyroid | Cytoplasmic |
| HepPar-1 | Hepatocellular | Cytoplasmic |
| CDX2 | Colorectal/duodenal | Nuclear |
| Villin | Gastrointestinal (epithelia | Apical |
| | with brush border) | |
| ER/PR | Breast, ovary, endometrium | Nuclear |
| GCDFP-15 | Breast | Cytoplasmic |
| Mammaglobin | Breast | Cytoplasmic |
| RCC marker | Renal | Membranous |
| PSA | Prostate | Cytoplasmic |
| PAP | Prostate | Cytoplasmic |
| Uroplakin III | Urothelial | Membranous |
| Inhibin | Sex cord-stromal, adrenocortical | Cytoplasmic |
| Melan-A | Adrenocortical, melanoma | Cytoplasmic |
| Calretinin | Mesothelioma, sex cord-stromal, | Cytoplasmic/ |
| | adrenocortical | cytoplasmic |
| WT1 | Ovarian serous, mesothelioma, | Nuclear |
| | Wilms, desmoplastic small round cell | |
| Mesothelin | Mesothelioma | Cytoplasmic/ |
| | | membranous |
| D2-40 | Mesothelioma, lymphatic | Membranous |
| | endothelial cell marker | |

CK7 and CK20 can be used to differentiate between several epithelial neoplasms, as described in e.g., Bahrami et al. (Arch Pathol Lab Med. 2008 132:326-48 and others. The following human epithelial neoplasms (which must be S100/HMB45/CD45 negative to rule out melanoma (S100, HMB45) and hematopoietic (CD45) tumors) can be distinguished based on CK7/CK20 staining:

CK7+/CK20+: urothelial carcinoma (uroplakin+, thrombomodulin+, P63+ and CK5/6); pancreatic adenocarcinoma (CEA+, CA19-9+, MUC5-AC+, MUC-2−, CDX2 (variable)); ovarian mucinous carcinoma (MUC5−, AC+, MUC-2−, CDX2 (variable); adenocarcinoma of the bladder (thrombomodulin+, CDX2 (variable)); gastric adenocarcinomal; cholangiocarcinoma.

CK7+/CK20−: breast adenocarcinoma (ER/PR+, GCDFP+, mammaglobin+, CEA+); endometrial adenocarcinoma (vimentin+, ER/PR+, CEA−); endocervical adenocarcinoma (CEA+, vimentin−, ER/PR−); Ivarian serous carcinoma (WT1+, ER/PR+, mesothelin+CEA−); lung adenocarcinoma (TTF-1+, CEA+, CK5/6−, p63−); lholangioarcinoma (CEA+, CK19+, MOC31+, CA19-9+, CDX2 (variable), HepPar1−); lung small cell carcinoma (TTF-1+, NE markers+, P63−); mesothelioma (calretinin+, WT1+CK5/6+, thrombomodulin+, D2-40+, mesothelin+, p63−, CEA−, MOC31−, Ber−EP4−, TTF-1−); thyroid carcinoma (TTF-1+, thyroglobulin+, CEA−); squamous cell carcinoma of cervix; salivary gland tumor, urothelial carcinoma; pancreatic and gastric adenocarcinoma.

CK7−/CK20+: colorectal adenocarcinoma (CDX2+, CEA+, MUC-2+, MUC5-AC−); Merkel cell carcinoma (NE markers+); gastric adenocarcinoma.

CK7−/CK20−: prostate adenocarcinoma (PSA+, PAP+, CEA−, uroplakin, thrombomodulin−, p63−, CK5/6−); squamous cell carcinoma (P63+, CK5/6+, thrombomodulin+); renal cell carcinoma (vimentin+, RCC marker+, CD10+, CEA−); hepatocellular carcinoma (HepPar1+, pCEA+, CD10+, MOC31−, CK19−); adrenocorital carcinoma (inhibin+, calretinin+, melanA+, vimentin+, CEA−); nonseminoma germ cell tumors (PLAP+, EMA−); yolk sac tumor (AFP+, embryonal CA, OCT3/4+, CD30+); mesothelioma; lung small cell carcinoma; gastric adenocarcinoma.

Further tissue- and organ-specific markers that are useful for identifying the source of a tumor are listed below.

ER and PR (estrogen receptor and progesterone receptor) expression associated with hormone-responsive organs and their neoplasms, such as breast, ovary, and endometrium. ER can bs sed to distinguish between an endometrial (vimentin−/ER−/CEA−) and an endocervical (vimentin−/ER−/CEA−) adenocarcinoma.

Gross cystic disease fluid protein 15 (GCDFP-15) is a 15-kd secreted glycoprotein of various body fluids, including saliva, milk, and seminal fluid, is considered a marker of apocrine differentiation, with high specificity for breast carcinomas.

Thyroid Transcription Factor 1 (TTF-1) is a nuclear transcription factor that promotes embryogenic pulmonary and thyroid differentiation and is expressed by most, but not all, lung or thyroid neoplasms. Almost all small cell carcinomas adenocarcinomas from the lung express TTF-1.

Thyroglobulin is a highly specific marker for papillary carcinoma and thyroid follicular carcinoma. Thyroid carcinomas are generally positive for vimentin and cytokeratins but are negative for the CEA, TTF-1, melanocytic, vascular, myogenous, and lymphoid markers.

Hepatocyte paraffin 1 (HepPar-1) is a monoclonal antibody that stains a unknown cellular antigen in normal hepatocytes and can be used as a sensitive and relatively specific marker for hepatocellular carcinoma.

CDX2 is an intestine-specific transcription factor that can be used as a sensitive and specific marker for colorectal and duodenal adenocarcinomas in both primary and metastatic sites.

Villin is a cytoskeletal protein associated with brush border microvilli of the intestine and proximal renal tubular epithelium. This protein is a sensitive marker of gastrointestinal adenocarcinomas.

Carcinoembryonic antigen (CEA) is a glycoprotein that overexpressed by a variety of adenocarcinomas as well as gastrointestinal adenocarcinomas. The CEA status in various carconinomas is listed here: CEA positive: colonic/rectal adenocarcinoma, gastric and esophageal adenocarcinoma, pancreatic adenocarcinoma Biliary tract adenocarcinoma, endocervical adenocarcinoma, and lung adenocarcinoma; CEA negative: endometrial adenocarcinoma, renal cell carcinoma, prostate adenocarcinoma, ovarian serous carcinoma, adrenal carcinoma and mesothelioma.

MOC-31 is a cell surface glycoprotein found on the epithelial cells. This marker can be used to differentiate between adenocarcinoma (MOC-31 positive) and mesothelioma (MOC-31 negative), for example.

Neuroendocrine markers indicate neuroendocrine neoplasms, including those with epithelial lineage (neuroendocrine carcinomas) and those with neural derivation, such as paraganglioma/pheochromocytoma and neuroblastoma. The most reliable markers of neuroendocrine neoplasms, synaptophysin and chromogranin, have a comparable sensitivity and are typically used together as complementary reagents. CD56 is generally regarded as a broad-spectrum neuroendocrine marker and can also be used.

Mammaglobin is a mammary-specific member of the uteroglobin family, is overexpressed in human breast carcinoma.

Renal cell carcinoma (RCC) marker, a monoclonal antibody that binds to gp200a, a glycoprotein found in normal human proximal tubular brush border cells, is a marker for conventional and papillary renal cell carcinoma. A subset of breast and embryonal carcinoma can also be stained for RCC expression.

The prostate-specific markers Prostatic specific antigen (PSA) and prostatic acid phosphatase (PAP) are glycoproteins produced almost exclusively by the prostatic glandular epithelium. These proteins can be used to identify the prostatic origin of metastatic tumors.

Thrombomodulin is a cell surface glycoprotein that involved in the regulation of intravascular coagulation. This protein is expressed in the majority of primary or metastatic urothelial carcinomas.

Inhibin is a peptide hormone that is produced by ovarian granulosa cells and testicular Sertoli cells. This protein serves as a sensitive and specific marker for ovarian and testicular sex cord-stromal tumors. Inhibin is also a sensitive marker for adrenal cortical neoplasms and reliably differentiates cortical from medullary adrenal tumors.

Melan-A is an antigen on melanoma cells. Melan-A can be used as a marker for melanoma marker although it can also be used to identify adrenal cortical neoplasms and differentiate between cortical from medullary adrenal tumors.

Calretinin expression identifies malignant mesothelioma with a sensitivity approaching 100%. This protein additionally serves as a highly sensitive marker for sex cord-stromal tumors.

Mesothelin is a cell surface antigen that is highly expressed in normal mesothelial cells, mesotheliomas, and a number of other carcinomas.

WT1, in addition to being a marker for small round cell tumors, can also be used to identify epithelioid mesofthelioma and discriminate it from adenocarcinoma.

Some immunohistochemical stains can help recognize specific substances in cancer cells that influence a patient's prognosis and/or whether they are likely to benefit from certain drugs. For example, immunohistochemical staining is routinely used to check for estrogen receptors on breast cancer cells. Patients whose cells have these receptors are likely to benefit from hormone therapy drugs, which block the production or effects of estrogens.

Immunohistochemistry can also help determine which women with breast cancer are likely to benefit from drugs that block the growth-promoting effects of abnormally high levels of HER2 protein. As such, the HER2 status of a cell may be analyzed.

In situ hybridization

In in situ hybridization methods, a labeled nucleic acid probe (which can be DNA or RNA, for example) is hybridized with intact, permeabilized, cells or a tissue section in order to analyze (i.e., identify the presence of and/or localize) specific sequences in the DNA or RNA in the cell. In situ hybridization DNA can be used to analyze, for example, DNA (using “DNA in situ hybridization” or “DNA ISH”), e.g., nuclear DNA, or RNA (using “RNA in situ hybridization” or “RNA ISH”), e.g., mRNA or miRNA, in a cell. DNA ISH can be used to determine the structure of chromosomes, for example. RNA ISH (RNA in situ hybridization) can used to measure and localize RNAs in a cell. The probes used in ISH are typically fluorescent.

A variety of in situ hybridization methods have been described (see, e.g., Cui et al Front. Cell Dev. Biol. 2016 4: 89 and Ratan et al Cureus. 2017 9: e1325) and can be adapted herein. In some cases, ISH can be combined with immunohistochemistry to analyze both protein and nucleic in a cell (see, e.g., Kochan et al, Biotechniques. 2015 59: 209-12, and others).

In situ hybridization is well adapted for analysis of diseases caused by aneuploidy as well as for the analysis of tumor cells, because tumors are often associated with a chromosomal rearranged. In situ hybridization can also be used to detect certain infectious diseases.

Aneuploidy

A multiplex FISH panel with differentially labeled probes can be used for prenatal screening of common aneuploidies involving gains or losses of chromosomes X, Y, 13, 18, and 21. Illustrative copy number abnormalities that can be detected using the method include, but are not limited to, trisomy 21, trisomy 13, trisomy 18, trisomy 16, XXY, XYY, XXX, monosomy X, monosomy 21, monosomy 22, monosomy 16, and monosomy 15. Further copy number abnormalities that can be detected using the present method are listed in the following table:

| Chromosome | Abnormality and Disease Association |
|---|---|
| X: | XO (Turner's Syndrome) |
| Y: | XXY (Klinefelter Syndrome) |
| Y: | XYY (Double Y Syndrome) |
| Y: | XXX (Trisomy X Syndrome) |
| Y: | XXXX (Four X Syndrome) |
| Y: | Xp21 deletion (Duchenne's/Becker Syndrome, |
| | congenital adrenal hypoplasia, chronic granulomatus |
| | disease) |
| Y: | Xp22 deletion (steroid sulfatase deficiency) |
| Y: | Xq26 deletion (X-linked lymphoproliferative disease) |
| 1: | 1p somatic (neuroblastoma) |
| 1: | monosomy (neuroblastoma) |
| 1: | trisomy (neuroblastoma) |
| 2: | monosomy (growth retardation, developmental and |
| | mental delay, and minor physical abnormalities) |
| 2: | trisomy 2q (growth retardation, developmental and |
| | mental delay, and minor physical abnormalities) |
| 3: | monosomy (Non-Hodgkin's lymphoma) |
| 3: | trisomy somatic (Non-Hodgkin's lymphoma) |
| 4: | monosomy (Acute non lymphocytic leukemia (ANLL)) |
| 4: | trisomy somatic (Acute non lymphocytic leukemia |
| | (ANLL)) |
| 5: | 5p (Cri du chat; Lejeune syndrome) |
| 5: | 5q somatic (myelodysplastic syndrome) |
| 5: | monosomy (myelodysplastic syndrome) |
| 5: | trisomy (myelodysplastic syndrome) |
| 6: | monosomy (clear-cell sarcoma) |
| 6: | trisomy somatic (clear-cell sarcoma) |
| 7: | 7q11.23 deletion (William's syndrome) |
| 7: | monosomy (monosomy 7 syndrome of childhood; |
| | somatic: renal cortical adenomas; |
| | myelodysplastic syndrome) |
| 7: | trisomy (monosomy 7 syndrome of childhood; somatic: |
| | renal cortical adenomas; myelodysplastic syndrome) |
| 8: | 8q24.1 deletion (Langer-Giedon syndrome) |
| 8: | monosomy (myelodysplastic syndrome; Warkany |
| | syndrome; somatic: chronic |
| | myelogenous leukemia) |
| 8: | trisomy (myelodysplastic syndrome; Warkany |
| | syndrome; somatic: chronic |
| | myelogenous leukemia) |
| 9: | monosomy 9p (Alfi's syndrome) |
| 9: | monosomy 9p (Rethore syndrome) |
| 9: | partial trisomy (Rethore syndrome) |
| 9: | trisomy (complete trisomy 9 syndrome; mosaic trisomy |
| | 9 syndrome) |
| 10: | monosomy (ALL or ANLL) |
| 10: | trisomy somatic (ALL or ANLL) |
| 11: | 11p- (Aniridia; Wilms tumor) |
| 11: | 11q- (Jacobsen Syndrome) |
| 11: | monosomy (myeloid lineages affected (ANLL, MDS)) |
| 11: | trisomy somatic (myeloid lineages affected (ANLL, |
| | MDS)) |
| 12: | monosomy (CLL, Juvenile granulosa cell tumor |
| | (JGCT)) |
| 12: | trisomy somatic (CLL, Juvenile granulosa cell tumor |
| | (JGCT)) |
| 13: | 13q- (13q-syndrome; Orbeli syndrome) |
| 13: | 13q14 deletion (retinoblastoma) |
| 13: | monosomy (Patau's syndrome) |
| 13: | trisomy (Patau's syndrome) |
| 14: | monosomy (myeloid disorders (MDS, ANLL, atypical |
| | CML) |
| 14: | trisomy somatic (myeloid disorders (MDS, ANLL, |
| | atypical CML) |
| 15: | 15q11-q13 deletion (Prader-Willi, Angelman's |
| | syndrome) |
| 15: | monosomy (Prader-Willi, Angelman's syndrome) |
| 15: | trisomy somatic (myeloid and lymphoid lineages |
| | affected, e.g., MDS, ANLL, ALL, CLL) |
| 16: | 16q13.3 deletion (Rubenstein-Taybi) |
| 16: | monosomy (papillary renal cell carcinomas (malignant)) |
| 16: | trisomy somatic (papillary renal cell carcinomas |
| | (malignant)) |
| 17: | 17p- somatic (17p syndrome in myeloid malignancies) |
| 17: | 17q11.2 deletion (Smith-Magenis) |
| 17: | 17q13.3 (Miller-Dieker) |
| 17: | monosomy (renal cortical adenomas) |
| 17: | trisomy somatic (renal cortical adenomas) |
| 17: | 17p11.2-12 (Charcot-Marie Tooth Syndrome type 1; |
| | HNPP) |
| 17: | trisomy (Charcot-Marie Tooth Syndrome type 1; HNPP) |
| 18: | 18p- (18p partial monosomy syndrome or Grouchy |
| | Lamy Thieffry syndrome) |
| 18: | 18q- (Grouchy Lamy Salmon Landry Syndrome) |
| 18: | monosomy (Edwards syndrome) |
| 18: | trisomy (Edwards syndrome) |
| 19: | monosomy (Edwards syndrome) |
| 19: | trisomy (Edwards syndrome) |
| 20: | 20p- (trisomy 20p syndrome) |
| 20: | 20p11.2-12 deletion (Alagille) |
| 20: | 20q- (somatic: MDS, ANLL, polycythemia vera, chronic |
| | neutrophilic leukemia) |
| 20: | monosomy (papillary renal cell carcinomas (malignant)) |
| 20: | trisomy somatic (papillary renal cell carcinomas |
| | (malignant)) |
| 21: | monosomy (Down's syndrome) |
| 21: | trisomy (Down's syndrome) |
| 22: | 22q11.2 deletion (DiGeorge's syndrome, |
| | velocardiofacial syndrome, conotruncal anomaly face |
| | syndrome, autosomal dominant Opitz G/BBB |
| | syndrome, Caylor cardiofacial syndrome) |
| 22: | monosomy (complete trisomy 22 syndrome) |
| 22: | trisomy (complete trisomy 22 syndrome) |

Oncology

In situ hybridization can be used to detect a variety of tumor-specific chromosome rearrangements, e.g., translocations that produce a fusion of an oncogenic kinase gene with another gene.

For example, results from a multiplexed in situ hybridization panel can identify chromosomal abnormalities associated with a number of hematologic cancers, including those of bone marrow cells or leucocytes, as well as number of solid tumors. For example, acute promyelocytic leukemia (APL) can be detecting an PML/RARa fusions, caused by a (15; 17)(q24; q21) translocation. ABL1/BCR fusions can also be detected. Other rearrangements that are undetectable by routine chromosome analysis, such as t(12; 21)(p13; q22) with ETV6/RUNX1 gene fusions, t(4; 14)(p16.3; q32) with FGFR3/IGH gene fusions, deletions of 12p13 (ETV6), 13q14 (RB1), and 17p13 (TP53) can be detected by ISH. Further examples are discussed below.

Chronic Myeloid Leukemia (CML): The BCR/ABL1 translocation often occurs in chronic myeloid leukemia (CML). ISH is currently used as the gold standard for detecting this chromosomal translocation.

Multiple Myeloma (MM): These cancers are a heterogeneous malignancy of terminally differentiated B cells. Four chromosomal partners appear to account for the majority of these cancers: 11q13 (cyclin D1), 6p21 (cyclin D3), 4p16 (FGFR3 and MMSET), and 16q23 (c-maf). These translocations can be detected using ISH.

Pulmonary Adenocarcinoma: Anaplastic lymphoma kinase (ALK) rearrangements are associated with pulmonary adenocarcinomas. For example, ALK rearrangements are mostly found from the fusion of the echinoderm microtubule-associated protein like 4 (EML4) with ALK at chromosome 2p23 [47]. EML4-ALK gene fusion can be detected through ISH.

Prostate Cancer: In approximately half of prostate cancers are associated with a fusion of TMPRSS2 and an E26 transformation-specific (ETS) family member (ERG, RTV1, and ETV4). In many cases, the chromosome rearrangement involves the fusion of TMPRSS2 to the oncogene ETS-related gene (ERG), which leads to the abnormal activation of ERG.

Breast Carcinomas: In some human breast carcinomas, overexpression of HER2 is observed. This can occurs through duplication of HER2. The HER2 status is often important for recommending a targeted therapy. For example, Herceptin-targeted therapy is effectively against HER2 over-expressed breast cancer. For routine clinical specimen, immunohistochemistry, real-time polymerase chain reaction, and ISH can be used to assess the HER2 protein level, RNA expression, and DNA copy numbers, respectively. Among these methods, ISH can offer a cell-based evaluation for the ratio of HER2 gene copy number to the number of copies of chromosome 17 (HER2/CEP17 ratio).

Renal Mesenchymal Neoplasm is often associated with a Xp11 translocation and, as such, can be detected by ISH.

Cholangiocarcinoma (CC): Cholangiocarcinoma a malignancy of the gastrointestinal tract and is often associated with a FGFR2 rearrangement. Cholangiocarcinoma can be detected using ISH.

Melanoma: Cholangiocarcinoma is a type of cancer that develops from the pigment-containing cells known as melanocytes. This can can be accurately detected through ISH.

For the diagnosis, four probes targeting 6p25 (RREB1), 6q23 (MYB), 11q13 (CCND1), and centromere 6 (CEP6) can be used.

Probes to the following genes can be used detect a variety of hematological cancers, including myeloid leukemia (including CML, MSD and AML), lymphocytic leukemia (including CLL, B-ALL and T-ALL), lymphoma, multiple myeloma and myeloproliferative disorder): CKS1B (1q21), CDKN2C (1p32), PBX1 (1q23.3), TCF3 (19p13.3), ALK (2p23), MECOM (3q26) BCL6 (3q27), D4Z1 (4cen), D10Z1 (10cen), D17Z1 (17cen), PDGFRA (4q12), FGFR3 (4p16.3), IGH (14q32), TAS2R1 (5p15.31), EGR1 (5q31), PDGFRB (5q33), MYB (6q23), D6Z1 (6cen), RELN (7q22), TES (7q31), TCRB (7q34), FGFR1 (8p11), RUNX1T1 (8q21), RUNX1 (21q22), cMYC (8q24), cMYC (8q24), D20S108 (20q12), PAX5 (9p13.2), CDKN2A (9p21), D9Z3 (9cen), ABL (9q34), BCR (22q11), CCND1 (11q13), IGH (14q32), ATM (11q22), TP53 (17p13), KMT2A (11q23), ETV6 (12p13), RUNX1 (21q22), DLEU1 (13q14), D13S25 (13q34), DLEU1 (13q14), D13S25 (13q34), D12Z3 (12cen), TCRA/D (14q11), IGH (14q32), IGH (14q32), BCL2 (18q21), SNRPN (15q11.2), TP53 (17p13), PML (15q24), RARA (17q21), MYH11 (16p13), CBFB (16q22), MALT1 (18q21) and CRLF2 (Xp22.33).

miRNA Analysis

miRNA molecules bind to specific target sequences within mRNA to effect post-translational gene regulation of cellular processes such as development, differentiation, proliferation, metabolism, and apoptosis (see, e.g., Tan et al. Oncol Lett. 2018 15: 2735-42 and Lin et al Nat Rev Cancer. 2015 15: 321-33). miRNA levels have been demonstrated to have a strong correlation with disease progression in cancer, cardiovascular disease, neurodegeneration, drug treatment, and numerous other pathologies (see, e.g., Chaudhuri et al FASEB J. 2013: 27, 3720-3729; Gambardella et al J. Transl. Med. 2010 8; Kluiver et al J. Pathol. 2005 207: 243-249; Kong et al Mol. Cell. Biol. 2008 28: 6773-6784; Liu et al Neurobiol. Aging 2012 33: 522-534; Wang et al FASEB J. 2008 22: 4126-4135 and Graveel et al, Breast Cancer 2015 7: 59-79). In particular, dysregulated cell signaling in many tumor types results in upregulation or downregulation of various miRNAs. miRNAs therefore have a powerful diagnostic, prognostic and theranostic utility. For example, microRNAs can accurately identify cancer tissue origin. (see Rosenfeld Nat Biotech. 2008 26:462-9)

Two advantages of miRNAs is that they are relatively straightforward to detect and their detection is highly specific. Due to their small size, miRNAs are resistant to degradation and chemical modification in fixed tissues and they are thought to correlate with expression levels in frozen tissues and body fluids, better than mRNAs (see, e.g., Szafranska et al. J Mol Diagn. 2008 10:415-23). These properties make miRNAs as ideal biomarkers, particularly for detecting cancer types and subtypes.

Most methods to measure miRNAs require total RNA extraction which lacks critical spatial information and present challenges for standardization. However, in situ hybridization (ISH) in fixed cells (e.g., FFPE tissue sections), allows precise detection and localization of specific nucleic acid sequences within a section, and preserves the anatomical structure of the fixed tissue (see, e.g., Szafranska et al J Mol Diagn. 2008 10: 415-23 and Nielsen Methods Mol Biol. 2012 822: 67-84. microRNA in situ hybridization or “miRNA-ISH” has become a powerful diagnostic, particularly for cancer due to the high level of sensitivity, accuracy, and ability to differentiate between single nucleotide mismatches. For example, among other things microRNAs can be used to differentiate malignant from benign tumors, for early-stage cancer detection marker, to identify cancer of unknown primary (CUP), to differentiate between cancer subtypes. miRNA expression profiles are also capable of grading, staging of cancer subtypes, and classifying undifferentiated and poorly differentiated tumors.

The following sections describes some of the steps that may be used in the present method.

Probe Design

In many embodiments, a probe used in the present method may be directly labeled, i.e., directly attached to a label such as a fluorescent label. Alternatively, a probe may be linked to a signal enhancement moiety such as an enzyme, e.g., alkaline phosphatase (AP). AP can be used in conjunction with nitro blue tetrazolium/5-bromo-4-chloro-3-indolyl phosphate (NBT/BCIP) or tyramide signal amplification (TSA), for example.

Modified DNA and RNA are commonly used for miRNA-ISH. In many embodiments, Locked Nucleic Acid (LNA) probes may be use. LNA nucleotides, which are sometimes referred to as “locked” RNA, have an additional bridge connecting 4′C and 2′O atoms. LNA nucleotides can be incorporated into DNA probes, thereby producing hybrid LNA/DNA probes. Such probes probes have been shown to be beneficial in miRNA detection because they have a short hybridization time, high efficiency, discriminatory power and a high melting temperature of the miRNA:probe complex. The minimal length of the LNA/DNA probe is 12 nucleotides and such probes usually contain 30% LNA nucleotides. Other modifications were also proposed include 2′fluoro-modified RNA (2′F RNA), morpholino, Zip Nucleic Acids (ZNA) [57], N,N-diethyl-4-(4-nitronaphthalen-1-ylazo)-phenylamine (ZEN) and 2′O-Methyl (2′OMe) RNA modifications. In comparison to DNA probes, 2′OMe RNA probes can have faster hybridization kinetics and the ability to bind structured targets under standard conditions. The combination of 2′OMe RNA and LNA modifications can result in improved specificity and stability of the probe:RNA duplex in comparison to the LNA/DNA probe. Specificity of the system may be further improved by shortening the probe length to 19 nt. 2′F RNA nucleotides may be incorporated in the DNA probes to ensure increased binding to the target and better nuclease resistance. Morpholino modifications, often applied to inhibit translation, modify splicing patterns of the primary transcript, or block miRNAs, were also used to detect miRNAs because of their high stability. The hybridization step itself may proceeds as in a standard in situ hybridization protocol

### Fixation

In some embodiments, the cells may be fixed before hybridization. Fixation should should sufficiently preserve the number and localization of mrRNA molecules, but, on the other hand, is mild enough to preserve cellular domains crucial for detection, particularly if the ISH is going to be combined with protein labeling. Prevention of miRNA loss during fixation is essential, especially for detecting low abundant miRNAs. Highly abundant miRNAs can be successfully detected using standard fixation protocols. In some embodiments, the cells may be fixed with 1-ethyl-3-(3-dimethylaminopropyl) carbodiimide (EDC) to immobilize miRNA molecules via their 5′ end. EDC fixation needs to be used in combination with traditional formaldehyde fixation. Improvement in the signal to noise ratio by addition of an EDC-crosslinking step was confirmed in several studies of miRNAs. miRNAs are also preserved through formalin-fixation and paraffin-embedding (FFPE) as well as cryopreservation.

Permeabilization

In order to facilitate permeabilization of the cells to the probe, the cells may be permeabilized. Permeabilization can be done using organic solvents including methanol or paraformaldehyde that are used also as fixatives, detergent like Saponin or Triton X-100, or proteinase. Briefly, organic solvents dissolve lipids from cell membranes. Some detergents, i.e., Saponin, remove cholesterol from membranes in highly selective ways, but widely used Triton X-100 is not selective, which can lead to elimination of both proteins and lipids. To reduce cellular RNA diffusion, treatment with proteinases and detergents was either limited or eliminated in some embodiments. The need for additional permeabilization decreases with the use of EDC fixation due to its auxiliary permeabilization activity. Tissues can be permeabilized using Triton X-100, but in some cases Proteinase K can be used additionally. However, permeabilization conditions may vary for the specific types of tissues, e.g., embryos or brain tissues and depend also on method of tissue preservation. Unfixed cryosections may not need permeabilization in contrary to fixed paraffin embedded sections. Cell lines may be permeabilized using acetone, Triton X-100, Tween or 70% ethanol. In some cases, the fixation and permeabilization steps may be combined.

miRNA Markers

The present system and method may be used to detect and/or analyze cancer (see, e.g., Oom et al Biomed Res Int. 2014 2014 959461), metabolic diseases (see, e.g., Nishiguchi et al Biomed Res Int. 2015 682857), cardiovascular disease (see, e.g., Peng et al, Cell Signal. 2014; 26:1888-1896) and neurodegenerative disorders (see, e.g., Maciotta et al., Front Cell Neurosci. 2013 7:265), among other things.

miRNA-Based Cancer Diagnostics

Many examples of miRNAs that can be used as cancer markers are known Three examples are described below.

miR-21 is one of the most well characterized miRNAs and is overexpressed in a number of solid tumors, including colon, breast, lung, pancreatic, prostate, bile duct, and stomach cancers (see, e.g., Volinia et al. Proc. Natl. Acad. Sci. 2006; 103: 2257-61). miR-21 is expressed in cancer-associated stromal fibroblasts and that the frequency and extent of miR-21 expression increased during the transition from precancerous colorectal adenoma to advanced carcinoma (see, e.g., Yamamichi et al Clin. Cancer Res. 2009; 15: 4009-16). miR-21 predicted limited survival in patients with node-negative disease and may prove be an important biologic marker for outcome (see, e.g., Dillhoff J. Gastrointest. Surg. 2008; 12: 2171-76). miR-21-positive tumor specimens were associated with poor prognosis.

miR-155 is another oncogenic miRNA that is overexpressed in several types of human solid tumors, including breast, colon, and lung cancer (see, e.g., Volinia et al. Proc. Natl. Acad. Sci. 2006; 103: 2257-61). They found that high miR-155 expression was an independent negative prognostic factor in adenocarcinomas and an independent favorable prognosticator in squamous cell carcinomas patients with regional nodal metastasis (Donnem et al. J. Transl. Med. 2011; 9: 6). miR-155 is also overexpressed (with miR-21, and miR-221) in invasive pancreatic cancer (see Ryu et al. Pancreatolog. 2010; 10: 66-73).

miR-10b is another oncogenic miRNA. miR-10b is the most frequently and consistently overexpressed miRNA among characterized miRNAs in pancreatic ductal adenocarcinoma (PDAC) samples, and its expression is increased in cancer cells compared with CK19-positive epithelial cells in benign lesions. In patients with PDAC, lower levels of miR-10b were associated with improved response to multimodality neoadjuvant therapy, likelihood of surgical resection, delayed time to metastasis, and increased survival. These findings suggest that expression of miR-10b is predictive of response to neoadjuvant therapy and outcome in this disease (Preis et al. Clin. Cancer Res. 2011; 17: 5812-21.)

miRNA-Based Diagnostics for Cardiovascular Diseases

miRNAs play an essential role in cardiovascular biology and are often dysregulated in several heart diseases, including myocardial hypertrophy, heart failure, and arrhythmia.

Deregulation of miR-1 and miR-133 has been associated with cardiac hypertrophy and arrhythmia (see, e.g., Catalucci et al, Circ. Cardiovasc. Genet. 2009; 2: 402-8) and the levels of miR-1, miR-133a, miR-208a, and miR-499 are significantly reduced in the infarcted myocardium Malizia et al, Wiley. Interdiscip. Rev. Syst. Biol. Med. 2011; 3: 183-90). miR-133a levels were very low in the infarcted and peri-infarcted myocardium, and it has been demonstrated that miR-133 is robustly expressed in vascular smooth muscle cells (VSMCs), whereas miR-1 is almost absent. Similarly, miR-133a is mainly expressed in VSMC cytoplasm, whereas miR133b is not expressed at all in VSMCs. miR-133 expression is regulated by extracellular signal-regulated kinase 1/2 activation and is inversely correlated with VSMC growth (see, e.g., Kuwabara et al, Circ. Cardiovasc. Genet. 2011; 4: 446-54.

myotonic dystrophy type 1 has been correlated with overexpression of miR-206 in the skeletal muscle (see, e.g., Gambardella et al, J. Transl. Med. 2010; 8: 48) and the same molecule has been localized to nucleus in both normal and DM1 tissues. However, in DM1 muscles, a strong signal was detected also in correspondence to centralized nuclei and nuclear clumps, which are pathological hallmarks of dystrophic muscles.

miRNA-Based Diagnostics for Neurodegenerative Diseases

Some miRNAs are aberrantly expressed in the brains of patients with neurodegenerative disorders, such as Huntington's disease (HD), Parkinson's disease (PD), and Alzheimer's disease (AD) [Junn et al, Pharmacol. Ther. 2012 133: 142-50).

AD is the most common dementia among aged people. A subset of miRNAs seemed to be specifically altered in the AD brain. Such “AD-specific” miRNAs include miR-29, miR-15, miR-16, miR-107, miR-9, miR-181, miR-101, miR-146, and miR-106 (see, e.g., Nelson et al, Brain Pathol. 2008; 18: 130-8 and others). The ISH staining showed that miR-107, miR124, miR-125b, and miR-320 have characteristic expression patterns in the human entorhinal cortex (EC) and transentorhinal cortex (TEC). The TEC is the area of the cerebral cortex that first develops neurofibrillary tangles in AD.

PD is the second most common neurodegenerative disorder and is characterized by the presence of protein inclusions, called Lewy bodies, and a progressive loss of dopaminergic neurons in the midbrain. Decreased expression of miR-34b and miR-34c has been found in in brain areas with variable neuropathological affects at different clinical stages of PD (Minones-Moyano et al. Hum. Mol. Genet. 2011 20: 3067-78).

HD is an autosomal dominant neurodegenerative disease caused by CAG trinucleotide repeat expansion in huntingtin, which encodes Huntingtin (Htt). miR-9 and miR-9* are decreased early in HD and are processed from the same primary transcript from three genomic loci (miR-9-1, miR-9-2, and miR-9-3) (see, e.g., Tan et al Genes Cells. 2012; 17: 952-61).

Qualitative and Quantitative Assessment

Depending on the biomarker, the method may be qualitative or quantitative. In some embodiments the method may be qualitative, e.g., when the biomarker is associated with an infectious agent and the biomarker is either present or absent. In some embodiments, the sub-cellular location of the biomarker within the cell may be determined (e.g., whether it is in the nucleus or cytoplasm). In other embodiments, the method may be quantitative. In these embodiments, two analytes may be detected: a biomarker and reference marker, where the signal obtained for the biomarker is normalized relative to the signal obtained for the reference marker. In these embodiments, the method may be performed in a multiplexed way, where the biomarker may be labeled with a first label (e.g., a first fluorophore) and the reference marker may be labeled with a second label that is distinguishable from the first label. Normalizing in such a way will allow cell-to-cell or sample-to-sample comparisons.

“Q-Cards”

As noted above, in some embodiments the method may be done by sandwiching a sample and a probe between the two plates to form a thin layer, referred to herein as a “sample-probe thin layer”, in the cell is in a monolayer. This may be conveniently done using a “Q-Card”, which comprises a first plate, a second plate, and spacers, wherein:
the plates are movable relative to each other into different configurations;
one or both plates are flexible;
each of the plates has, on its respective surface, a sample contact area for contacting a sample that contains an analyte,
one or both of the plates comprise the spacers that are fixed with a respective sample contact area, wherein the spacers have a pillar shape, a substantially flat top surface, a predetermined substantially uniform height and a predetermined inter-spacer distance, wherein at least one of the spacers is inside the sample contact area, wherein the inter-spacer distance is a distance between two neighboring spacers, and wherein the Young's modulus of the spacers times the filling factor of the spacers is equal or larger than 2 MPa;
wherein one of the configurations is an open configuration, in which: the two plates are separated apart, the spacing between the plates is not regulated by the spacers, and the sample is deposited on one or both of the plates; and
wherein another of the configurations is a closed configuration which is configured after the sample deposition in the open configuration; and in the closed configuration: at least part of the sample is compressed by the two plates into a layer of highly uniform thickness, wherein the uniform thickness of the layer is confined by the sample contact surfaces of the plates and is regulated by the plates and the spacers. Q-cards are described in US20180202903, which patent application is incorporated by reference herein for all description relating to the same.

In some embodiments, the Q-card comprises spacers, which help to render at least part of the sample into a layer of high uniformity. The structure, material, function, variation and dimension of the spacers, as well as the uniformity of the spacers and the sample layer, are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application No. 62/456,287, which was filed on Feb. 8, 2017, and U.S. Provisional Application No. 62/456,504, which was filed on Feb. 8, 2017, all of which applications are incorporated herein in their entireties for all purposes.

The term “open configuration” of the two plates in a Q-Card means a configuration in which the two plates are either partially or completely separated apart and the spacing between the plates is not regulated by the spacers

The term “closed configuration” of the two plates in a Q-Card means a configuration in which the plates are facing each other, the spacers and a relevant volume of the sample are between the plates, the relevant spacing between the plates, and thus the thickness of the relevant volume of the sample, is regulated by the plates and the spacers, wherein the relevant volume is at least a portion of an entire volume of the sample.

The term “a sample thickness is regulated by the plate and the spacers” in a Q-Card means that for a give condition of the plates, the sample, the spacer, and the plate compressing method, the thickness of at least a port of the sample at the closed configuration of the plates can be predetermined from the properties of the spacers and the plate.

The term “inner surface” or “sample surface” of a plate in a Q-Card refers to the surface of the plate that touches the sample, while the other surface (that does not touch the sample) of the plate is termed “outer surface”.

The term “height” or “thickness” of an object in a Q-Card refers to, unless specifically stated, the dimension of the object that is in the direction normal to a surface of the plate.

For example, spacer height is the dimension of the spacer in the direction normal to a surface of the plate, and the spacer height and the spacer thickness means the same thing.

The term “area” of an object in a Q-Card refers to, unless specifically stated, the area of the object that is parallel to a surface of the plate. For example, spacer area is the area of the spacer that is parallel to a surface of the plate.

A Q-Card may or may not have a hinge that connect the two plates.

The term “angle self-maintain”, “angle self-maintaining”, or “rotation angle self-maintaining” refers to the property of the hinge, which substantially maintains an angle between the two plates, after an external force that moves the plates from an initial angle into the angle is removed from the plates.

In using a Q-Card, the two plates need to be open first for sample deposition.

However, in some embodiments, a Q-Card from a package has the two plates are in contact each other (e.g. a close position), and to separate them is challenges, since one or both plates are very thing. To facilitate an opening of the Q-Card, an opening notch or notches may be created at the edges or corners of the first plate or both places, and, at the close position of the plates, a part of the second plate placed over the opening notch, hence in the notch of the first plate, the second plate can be lifted open without a blocking of the first plate.

In a Q-Card, two plates can be used to manipulate the shape of a sample into a thin layer (e.g. by compressing). In certain embodiments, the plate manipulation needs to change the relative position (termed: plate configuration) of the two plates several times by human hands or other external forces. There

In using a Q-Card, one of the plate configurations is an open configuration, wherein the two plates are completely or partially separated (the spacing between the plates is not controlled by spacers) and a sample can be deposited. Another configuration is a closed configuration, wherein at least part of the sample deposited in the open configuration is compressed by the two plates into a layer of highly uniform thickness, the uniform thickness of the layer is confined by the inner surfaces of the plates and is regulated by the plates and the spacers. In some embodiments, the average spacing between the two plates is more than 300 μm.

In use of a Q-Card, an operator should first place the two plates in an open configuration ready for sample deposition, then deposit a sample on one or both of the plates, and finally close the plates into a close position. In certain embodiments, the two plates of a Q-Card are initially on top of each other and need to be separated to get into an open configuration for sample deposition. When one of the plate is a thin plastic film (e.g., 175 μm thick PMA), such separation can be difficult to perform by hand. of certain assays, such as the QMAX card assay, easy and fast.

In some embodiments, the Q-Card comprises a hinge that connect the plates together, so that the plates can open and close in a similar fashion as a book. In some embodiments, the material of the hinge is such that the hinge can self-maintain the angle between the plates after adjustment. In some embodiments, the hinge is configured to maintain the Q-Card in the closed configuration, such that the entire Q-Card can be slide in and slide out a card slot without causing accidental separation of the two plates. In some embodiments, the Q-Card comprises one or more hinges that can control the rotation of more than two plates.

In some embodiments, the hinge is made from a metallic material that is selected from a group consisting of gold, silver, copper, aluminum, iron, tin, platinum, nickel, cobalt, alloys, or any combination of thereof. In some embodiments, the hinge comprises a single layer, which is made from a polymer material, such as but not limited to plastics. The polymer material is selected from the group consisting of acrylate polymers, vinyl polymers, olefin polymers, cellulosic polymers, noncellulosic polymers, polyester polymers, Nylon, cyclic olefin copolymer (COC), poly(methyl methacrylate) (PMMB), polycarbonate (PC), cyclic olefin polymer (COP), liquid crystalline polymer (LCP), polyamide (PB), polyethylene (PE), polyimide (PI), polypropylene (PP), poly(phenylene ether) (PPE), polystyrene (PS), polyoxymethylene (POM), polyether ether ketone (PEEK), polyether sulfone (PES), poly(ethylene phthalate) (PET), polytetrafluoroethylene (PTFE), polyvinyl chloride (PVC), polyvinylidene fluoride (PVDF), polybutylene terephthalate (PBT), fluorinated ethylene propylene (FEP), perfluoroalkoxyalkane (PFB), polydimethylsiloxane (PDMS), rubbers, or any combinations of thereof. In some embodiments, the polymer material is selected from polystyrene, PMMB, PC, COC, COP, other plastic, or any combination of thereof.

In essence, the term “spacers” or “stoppers” refers to, unless stated otherwise, the mechanical objects that set, when being placed between two plates, a limit on the minimum spacing between the two plates that can be reached when compressing the two plates together. Namely, in the compressing, the spacers will stop the relative movement of the two plates to prevent the plate spacing becoming less than a preset (i.e. predetermined) value.

The term “a spacer has a predetermined height” and “spacers have a predetermined inter-spacer distance” means, respectively, that the value of the spacer height and the inter spacer distance is known prior to a use. It is not predetermined, if the value of the spacer height and the inter-spacer distance is not known prior to use. For example, in the case that beads are sprayed on a plate as spacers, where beads are landed at random locations of the plate, the inter-spacer distance is not predetermined. Another example of not predetermined inter spacer distance is that the spacers moves during use.

The term “a spacer is fixed on its respective plate” in a Q-Card means that the spacer is attached to a location of a plate and the attachment to that location is maintained during use (i.e. the location of the spacer on respective plate does not change) process. An example of “a spacer is fixed with its respective plate” is that a spacer is monolithically made of one piece of material of the plate, and the location of the spacer relative to the plate surface does not change during the use. An example of “a spacer is not fixed with its respective plate” is that a spacer is glued to a plate by an adhesive, but during a use of the plate, during use, the adhesive cannot hold the spacer at its original location on the plate surface and the spacer moves away from its original location on the plate surface.

In some embodiments, human hands can be used to press the plates into a closed configuration; In some embodiments, human hands can be used to press the sample into a thin layer. The manners in which hand pressing is employed are described and/or summarized in PCT Application (designating U.S.) Nos. PCT/US2016/046437 filed on Aug. 10, 2016 and PCT/US2016/051775 filed on Sep. 14, 2016, and in US Provisional Application Nos. 62/431,639 filed on Dec. 9, 2016, 62/456,287 filed on Feb. 8, 2017, 62/456,065 filed on Feb. 7, 2017, 62/456,504 filed on Feb. 8, 2017, and 62/460,062 filed on Feb. 16, 2017, which are all hereby incorporated by reference by their entireties.

In some embodiments, human hand can be used to manipulate or handle the plates of the Q-Card. In certain embodiments, the human hand can be used to apply an imprecise force to compress the plates from an open configuration to a closed configuration. In certain embodiments, the human hand can be used to apply an imprecise force to achieve high level of uniformity in the thickness of the sample (e.g. less than 5%, 10%, 15%, or 20% variability).

For example, in some embodiments, the spacers regulating the layer of uniform thickness (i.e., the spacers that are spacing the plates away from each other in the layer) have a “filling factor” of at least 1%, e.g., at least 2% or at least 5%, wherein the filling factor is the ratio of the spacer area that is in contact with the layer of uniform thickness to the total plate area that is in contact with the layer of uniform thickness. In some embodiments, for spacers regulating the layer of uniform thickness, the Young's modulus of the spacers times the filling factor of the spacers is equal or larger than 10 MPa, e.g., at least 15 MPa or at least 20 MPa, where the filling factor is the ratio of the spacer area that is in contact with the layer of uniform thickness to the total plate area that is in contact with the layer of uniform thickness. In some embodiments, the thickness of the flexible plate times the Young's modulus of the flexible plate is in the range of 60 to 550 GPa-μm, e.g., 100 to 300 GPa-μm. In some embodiments, for a flexible plate, the fourth power of the inter-spacer-distance (ISD) divided by the thickness of the flexible plate (h) and the Young's modulus (E) of the flexible plate, ISD⁴/(hE), is equal to 5 or less than 10⁶ μm³/GPa, e.g., less than 105 μm³/GPa, less than 104 μm³/GPa or less than 103 μm³/GPa.

In some embodiments, one or both plates comprise a location marker either on a surface of or inside the plate, that provide information of a location of the plate, e.g., a location that is going to be analyzed or a location onto which the section should be deposited. In some cases, one or both plates may comprise a scale marker, either on a surface of or inside the plate, that provides information of a lateral dimension of a structure of the section and/or the plate. In some embodiments, one or both plates comprise an imaging marker, either on surface of or inside the plate, that assists an imaging of the sample. For example, the imaging marker could help focus the imaging device or direct the imaging device to a location on the device. In some embodiments, the spacers can function as a location marker, a scale marker, an imaging marker, or any combination of thereof.

In some embodiments, the inter-spacer distance may substantially periodic. In some cases, the spacers may be in a regular pattern and the spacing between adjacent spacers may be approximately the same. In some embodiments, the spacers are pillars with a cross-sectional shape selected from round, polygonal, circular, square, rectangular, oval, elliptical, or any combination of the same and, in some embodiments, the spacers may have a substantially flat top surface, wherein, for each spacer, the ratio of the lateral dimension of the spacer to its height is at least 1. In some cases, the minimum lateral dimension of spacer is less than or substantially equal to the minimum dimension of an analyte in the sample. The minimum lateral dimension of spacer is in the range of 0.5 μm to 100 μm, e.g., in the range of 2 μm to 50 μm or 0.5 μm to 10 μm.

In some embodiments, the spacers have a pillar shape and the sidewall corners of the spacers have a round shape with a radius of curverture at least 1 μm, e.g., at least 1.2 μm, at least 1.5 μm or at least 2.0 μm. The spacers may have any convenient density, e.g., a density of at least 1,000/mm², e.g., a density of at least 1,000/mm², a density of at least 2,000/mm², a density of at least 5,000/mm² or a density of at least 10,000/mm².

In any embodiment, the spacers on the Q-card may have a height of 0.5 um, 1 um, 2 um, 3 um, 5 um, 10 um, 15 um, 20 um, 30 um, 50 um, or a range between any two of the values. In some embodiments, the spacers on the Q-card have a height of 0.5 um, 1 um, 2 um, 3 um, 5 um, 10 um, 15 um, or a range between any two of the values.

In this device, at least one of the plates may be transparent, thereby allowing the assay to be read optically. Likewise, in this device, at least one of the plates may be made of a flexible polymer, thereby allowing the sample to be efficiently spread by compressing the plates together. In some embodiments, the pressure that compresses the plates, the spacers are not compressible and/or, independently, only one of the plates is flexible. The flexible plate may have a thickness in the range of 20 μm to 200 μm, e.g., 50 μm to 150 μm. As noted above, in the closed position, the thickness of the layer of uniform thickness may have a small variation.

In some embodiments, the variation may be less than 10%, less than 5% or less than 2%, meaning that the thickness of the area does not exceed +/−10%, +/−5% 5 or +/−2% of the average thickness.

In some embodiments, the first and second plates are connected and the device can be changed from the open configuration to the closed configuration by folding the plates. In some embodiments, the first and second plates can be connected by a hinge and the device can be changed from the open configuration to the closed configuration by folding the plates such that the device bends along the hinge. The hinge may be a separate material that is attached to the plates or, in some cases, the plates may be integral with the plates.

In some embodiments, the device may be capable of analyzing the section very rapidly. In some cases, the analysis may be done in 60 seconds or less, in 30 seconds, in 20 seconds or 15 less or in 10 seconds or less.

As noted above or below, in some embodiments, one or more plates of a Q-card may comprise a dry staining agent coated on the sample contact area.

The structure, material, function, variation and dimension of Q-cards, hinges, notches, recesses, and sliders are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/431,639, which was filed on Dec. 9, 2016, U.S. Provisional Application No. 62/456,065, which was filed on Feb. 7, 2017, U.S. Provisional Application Nos. 62/456,287 and 62/456,504, which was filed on Feb. 8, 2017, and U.S. Provisional Application No. 62/539,660, which was filed on Aug. 1, 2017, all of which applications are incorporated herein in their entireties for all purposes.

In some embodiments, the Q-card comprises opening mechanisms such as but not limited to notches on plate edges or strips attached to the plates, making is easier for a user to manipulate the positioning of the plates, such as but not limited to separating the plates of by hand.

In some embodiments, the Q-card comprises trenches on one or both of the plates. In certain embodiments, the trenches limit the flow of the sample on the plate.

In use, sample is placed on the one of the plates of a Q-card, e.g., 100 μL or less, 75 μL or less, 50 μL or less, 25 μL or less, 20 μL or less, 15 μL or less, 10 μL or less, 5 μL or less, 3 μL or less, 1 μL or less, 0.5 μL or less, 0.1 μL or less or an amount that is between any two of the values, the plates are moved together to sandwich the sample, and the signal is read after a period of time (which is typically less than a few minutes, e.g., less than 5 minutes, less than 4 minutes, less than 3 minutes, less than 2 minutes, or less than 1 minute).

### Systems and Mobile Phone Embodiments

Also provided is a system for performing the method. In some embodiments, the system may comprise or may make use of a mobile phone. In these embodiments, the system may comprise a Q card device as described above and (b) a mobile communication device (e.g., a mobile phone) comprising: i. one or more cameras for detecting and/or imaging the sample; ii. electronics, signal processors, hardware and software for receiving and/or processing the detected signal and/or the image of the sample and for remote communication; and (c) a light source from either the mobile communication device or an external source. In these embodiments, the method may be done by (a) depositing this sample (and, optionally a staining reagent if it is not already on the surface of a plate) on a plate and placing the two plate into a closed configuration; (b) imaging the sample to generate a result; and (c) communicating the result from the mobile phone to a location that is remote to the mobile phone.

In some embodiments, the mobile communication device may contain hardware and software that allows it to (a) capture an image of the sample; (b) analyze the image, in accordance with the method; and (c) output a determination of whether the analyte is in the sample, or a quantity of analyte. In any embodiment, the mobile communication device may be a mobile phone.

In some embodiments, the method may involve creating a report (an electronic form of which may have been forwarded from a remote location) and forwarding the report to a doctor or other medical professional to determine whether a patient has a phenotype (e.g., cancer, etc.) or to identify a suitable therapy for the patient. The report may be used as a diagnostic to determine whether the subject has a disease or condition, e.g., a cancer. In certain embodiments, the method may be used to determine the stage or type cancer, to identify metastasized cells, or to monitor a patient's response to a treatment, for example.

In any embodiment, report can be forwarded to a “remote location”, where “remote location,” means a location other than the location at which the image is examined. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like. In certain embodiments, the report may be analyzed by an MD or other qualified medical professional, and a report based on the results of the analysis of the image may be forwarded to the patient from which the sample was obtained.

Adaptors

In some embodiments, the Q-card may be used together with an adaptor that is configured to accommodate the Q-card and connect to a mobile device so that the sample in the Q-card can be imaged, analyzed, and/or measured by the mobile device. The structure, material, function, variation, dimension and connection of the Q-card, the adaptor, and the mobile are herein disclosed, listed, described, and/or summarized in PCT Application (designating U.S.) Nos. PCT/US2016/046437 and PCT/US2016/051775, which were respectively filed on Aug. 10, 2016 and Sep. 14, 2016, U.S. Provisional Application No. 62/456,065, which was filed on Feb. 7, 2017, U.S. Provisional Application Nos. 62/456,287 and 62/456,590, which were filed on Feb. 8, 2017, U.S. Provisional Application No. 62/456,504, which was filed on Feb. 8, 2017, U.S. Provisional Application No. 62/459,544, which was filed on Feb. 15, 2017, and U.S. Provisional Application Nos. 62/460,075 and 62/459,920, which were filed on Feb. 16, 2017, all of which applications are incorporated herein in their entireties for all purposes.

In some embodiments, the adaptor may comprise a receptacle slot, which is configured to accommodate the Q-card when the device is in a closed configuration. In certain embodiments, the Q-card has a sample deposited therein and the adaptor can be connected to a mobile device (e.g. a smartphone) so that the sample can be read by the mobile device. In certain embodiments, the mobile device can detect and/or analyze a signal from the sample. In certain embodiments, the mobile device can capture images of the sample when the sample is in the Q-card and positioned in the field of view (FOV) of a camera, which in certain embodiments, is part of the mobile device.

In some embodiments, the adaptor comprises optical components, which are configured to enhance, magnify, and/or optimize the production of the signal from the sample. In some embodiments, the optical components include parts that are configured to enhance, magnify, and/or optimize illumination provided to the sample. In certain embodiments, the illumination is provided by a light source that is part of the mobile device. In some embodiments, the optical components include parts that are configured to enhance, magnify, and/or optimize a signal from the sample.

Applications

The present method can be used to analyze samples from any organism, including eukaryotic, prokaryotic, animal, plant and microbial samples. In some embodiments, the sample can be from a human.

The following are examples of diagnostic methods that can be performed:

### Example 1: PAP (Papanicolaou) Testing

The present can be used in Pap tests, sometimes called Pap smears, which are tests for finding abnormal cells on patients' cervix that could lead to cervical cancer. Pap tests find cell changes caused by HPV, but they don't detect HPV itself. The present invention can make the PAP test in a single step and in 60 seconds.

One example of performing a Pap test using the present invention, includes steps of:
7. depositing a cell sample from a cervix onto a plate of a Q-card;
8. if the staining reagent is not dried onto the surface of one of the plates of the Q-card, then depositing a staining reagent on one of the plates of device or directly on the sample, when the plates are configured in an open configuration;
9. forcing the plate and substrate into a closed configuration, in which: at least part of the sample is compressed by the plate into a thin layer, wherein the thickness of the layer is partially regulated by the spacers on one of the plates;
10. without washing, after 1 to 5 minutes, imaging the sample in device with the imaging system;
11. analyzing the image with analyzing system.

The cells may be deposited onto the Q-card directly from a scraping device (e.g., a speculum) or the cells may be deposited into a liquid prior to being deposited onto the Q-card.

In some embodiments, the Pap test may be done using the FAST Papanicolaou staining method. In these embodiments, the Papanicolaou staining solution may comprise: hematoxylin, eosin azure (eosin Y, light green SF, Bismarck brown Y), orange green 6, alcohol, and water. These reagents may be dried onto one of the plates, if desired. The staining can be observed using the camera of a smartphone, or a light microscope.

Pap stains stain cells in the following way: nuclei: blue, acidophilic cells: red, basophilic cells: blue green, erythrocytes: orange-red, keratin: orange-red; superficial cells: pink, intermediate and parabasal cells: blue green, eosinophil: orange red, candida: red, trichomonas: grey green.

HPV infected cervical epithelial cells will show typical koilocytes with enlarged nuclei, irregular nuclear membrane contour, hyperchromasia (darker than normal staining pattern in the nucleu), and perinuclear halo (a clear area around the nucleus).

There are four types of abnormal FAST Pap smear results:
1. Atypical squamous cells of undetermined significance (ASCUS): These results indicate slightly abnormal squamous cells-thin, flat cells that grow on the surface of the cervix. Changes in these cells don't clearly suggest precancerous cells are present. With the liquid-based test, reanalyze the sample to check for the presence of viruses known to promote the development of cancer, such as some types of HPV. If no high-risk viruses are present, the abnormal cells found as a result of the test aren't of great concern. If worrisome viruses are present, a further testing is needed.
2. Squamous intraepithelial lesion: This term indicates that the sample cells may be precancerous. If the changes are described as low-grade squamous intraepithelial lesions (LSILS), it means the size, shape, and other characteristics suggest that if a precancerous lesion is present, it's likely to be years away from becoming cancer. High-grade squamous intraepithelial lesions (HSILS) may develop into cancer sooner. Additional diagnostic testing is necessary.
3. Atypical glandular cells (AGC): Glandular cells produce mucus and grow in the opening of the cervix and within the uterus. Atypical glandular cells may appear to be slightly abnormal, but it's unclear whether they're cancerous. Further testing is needed to determine the source of the abnormal cells and their significance.
4. Squamous cell carcinoma or adenocarcinoma cells: This result means the cells collected for the Pap smear appear so abnormal that the pathologist is almost certain a cancer is present. Squamous cell cancer refers to cancers arising in the flat surface cells of the vagina or cervix. Adenocarcinoma refers to cancers arising in glandular cells. If such cells are found, doctor will recommend a prompt evaluation.

### Example 2: Viral Detection

Detection of a viral infection can be done by, for example, identifying a viral-specific glycoprotein inside a cell of the subject, wherein a virus glycol-protein specific probe is introduced inside a cell (e.g. inside plasma and/or nucleus), wherein the probe has a label, wherein the probe concentration in the cell is configured so that at least one portion of the volume of the cell has a concentration of the probes bound to a specific virus glycol-protein higher than that of unbind probes in a portion of the volume of (i) other portion of the cell and/or (ii) other portion of a volume outside of the cell. In some embodiments, the staining reagent may comprise a permeabilizing agent capable of permeabilizing cells in the tissue sample that contain the target analyte.

### Urine Cytology

Urine cytology is a test to look for abnormal cells in urine samples. It's used with other tests and procedures to diagnose urinary tract infectious diseases, premalignant conditions and malignant diseases, most often bladder cancer. Although technologies have advanced the urine sample analysis at molecular level, urine cytology still gold method for the diagnosis of many diseases, especially cancers.

Traditional urine cytology methods often require sample preparation (for example cytospin centrifugation) and staining (for example Feulgen-Schiff staining), whole procedure will take longer than 15 min with multiple steps. We introduce a FAST Q-MAX card device that can shorten the whole procedure to less than 1 min with one step and give direct imaging and analysis simultaneously.

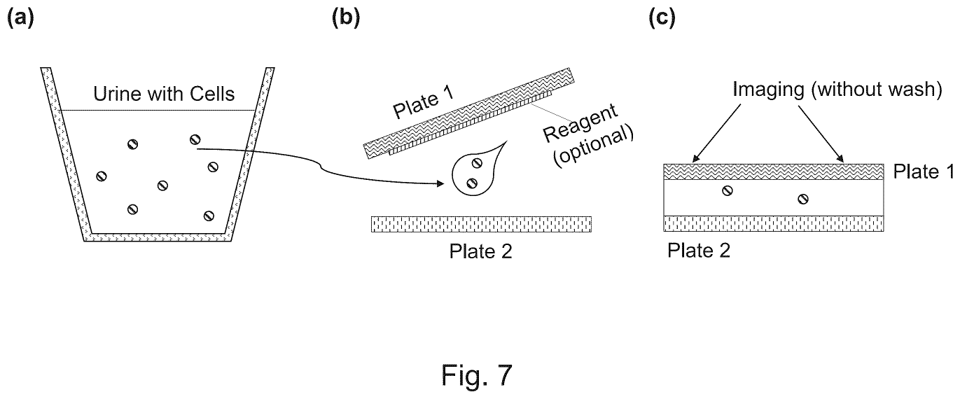

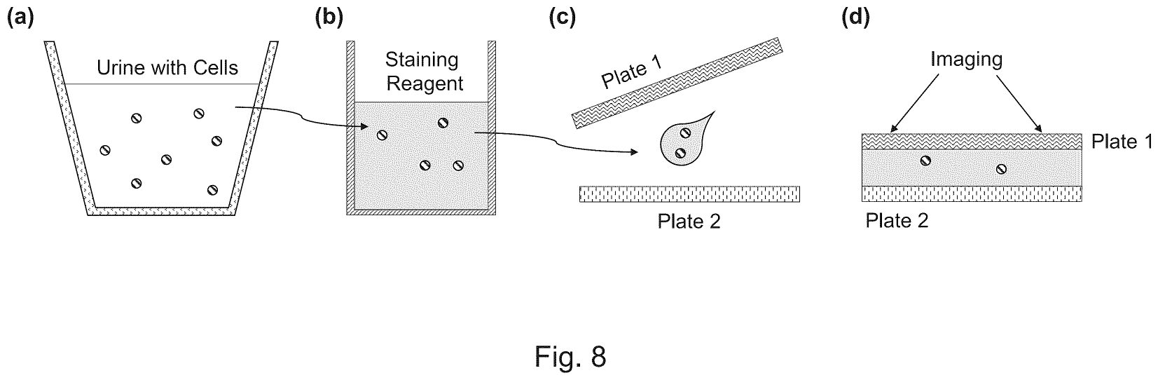

According the present invention, as illustrated in FIGS. 7 and 8, a method of fast staining and imaging the cells in the urine sample, comprising:
i) obtaining a device of above;
ii) depositing a urine sample on plate or substance; when the plates are configured in an open configuration, wherein the open configuration is a configuration in which the plate and substance are partially or completely separated apart and the spacing between is not regulated by the spacers;
iii) forcing the plate and substance into a closed configuration, in which: at least part of the sample is compressed by the plate into a layer of substantially uniform thickness, wherein the uniform thickness of the layer is confined by the sample contact surfaces of the plate and is regulated by the spacers;
iv) imaging the cells inside the device.

In some embodiments, a method of fast staining and imaging the cells in the urine sample, comprising the steps of:
i) obtaining a device of above;
ii) collecting the urine sample in a container;
iii) transferring a urine sample on plate or substance; when the plates are configured in an open configuration, wherein the open configuration is a configuration in which the plate and substance are partially or completely separated apart and the spacing between is not regulated by the spacers;
iv) forcing the plate and substance into a closed configuration, in which: at least part of the sample is compressed by the plate into a layer of substantially uniform thickness, wherein the uniform thickness of the layer is confined by the sample contact surfaces of the plate and is regulated by the spacers;
v) imaging the cells inside the device.

In some embodiments, a method of of fast staining and imaging the cells in the urine sample, comprising the steps of:
i) obtaining a device of above;
ii) collecting the urine sample in a container;
iii) staining the cells in the urine sample by mixing the urine sample with staining reagents;
iv) transferring a stained urine sample on plate or substance; when the plates are configured in an open configuration, wherein the open configuration is a configuration in which the plate and substance are partially or completely separated apart and the spacing between is not regulated by the spacers;
v) forcing the plate and substance into a closed configuration, in which: at least part of the sample is compressed by the plate into a layer of substantially uniform thickness, wherein the uniform thickness of the layer is confined by the sample contact surfaces of the plate and is regulated by the spacers;
vi) imaging the cells inside the device.

In some embodiments, a FAST Q-MAX card staining material, comprising:
1. Urine samples;
2. Q-Card device comprising bottom plate and a top X-plate;
3. Staining solution comprising dyes (fluorescent and/or coulometric), antibodies and nuclei acid probes to differentiate cells found in urine samples including urothelial cells, squamous epithelial cells, red blood cells, white blood cells (neutrophil, lymphocytes, eosinophils, histocytes, giant cells), as well as sperm, crystals, casts, various organisms, extraneous material.
4. IPHONE or light microscope.

In some embodiments, a procedure option comprising:
1. Drop 5-10 ul of urine sample directly onto bottom card of Q-Card device;
2. Close Q-Card and leave at room temperature for 1 min;
3. Observe and analyze using IPHONE or microscope.

FIG. 7 illustrates a flow chart of one exemplary embodiment of the method with the steps of (a) collecting urine samples containing cells using a container; (b) transferring a drop of urine sample onto one or both of the plates the device (i.e. Q-Card) in an open configuration; (c) closing the device and imaging, without a wash, the cell sandwiched by two plates (plate 1 and plate 2) of the Q-Card. In some embodiments, the gap (i.e. the spacing between the plates) and the concentration of reagent between the two plates are configured, so that at the closed configuration (i) cells in the sample forming a monolayer (i.e. no significant lateral overlap), and (ii) a stain cell is visible without washing. In some embodiments, staining reagents are coated on the plates (either dry reagent or a liquid reagent) and dissolved into the urine when in a closed configuration.

### Procedure Option 2:

1. Mix staining solution with urine sample in a separate tube;
2. Drop 5-10 ul of mixture solution onto bottom card of Q-Card device;
3. Close Q-Card and leave at room temperature for 1 min;
4. Observe and analyze using IPHONE or microscope.

FIG. 8 illustrates a flow chart of one exemplary embodiment of the method with the steps of (1) collecting urine samples with cells inside a container; (2) staining the cells in the urine sample by mixing staining reagents with urine sample, (3) transferring a drop of urine sample onto the device in open configuration; (4) closing the device and imaging the cell sandwiched by two plates (plate 1 and plate 2) with a gap naming “SNT”, which ensures (i) cells forming a monolayer and (ii) sub noise thick for imaging.

### Examples of Urine Cells and Potential Diseases.

| | |
|---|---|
| Urine cells | Urothelial cells: deep urothelial cells (basal and |
| | intermediate cells), superficial urothelial cells (umbrella |
| | cells), columnar cells, worrisome atypia in reactive/ |
| | degenerative cells, papillary and pseudopapillary clusters |
| | in voided urine; |
| | Other cells: renal tubular cells, squamous cells and |
| | squamous metaplasia, male cells and components, blood |
| | and inflammatory cells (red blood cell, neutrophils, |
| | lymphocytes, plasm cells, eosinophils, histocytes), |
| | melamed-wolinska bodies (hyaline inclusion bodies); |
| | Other findings: sperm, crystals, casts, various organisms, |
| | extraneous material |
| Benign | Lithiasis (stones); cystitis [(most common infection caused |
| urinary tract | by E. Coli., special infections (human polyomavirus, |
| diseases and | cytomegalovirus (CMV), herpesvirus, human |
| conditions | papillomavirus (HPV), tuberculosis (TB), fungus, parasites |
| | (schistosomiasis) miscellaneous organisms), other types of |
| | cystitis (eosinophilic cystitis, malakoplakia)]; |
| | intravenous and retrogradepyelogram effect (radiologic |
| | contrast material); heavy metal poisoning; renal transplant; |
| | therapeutic effects (chemotherapy: cyclophosphamide |
| | (cytoxan), busulfan, thiotepa, mitomycin, cyclosporine, |
| | nephrogenic adenoma |
| Urinary tract | Urothelial neoplasia (papilloma), urothelial carcinoma |
| cancer | (bladder cancer, papillary, flat/nodular, low grade and high |
| | grade, carcinoma in situ), leukoplakia, bladder condylomas, |
| | squamous cell carcinoma, adenocarcinoma, small cell |
| | carcinoma, sarcoma, mixed urothelial carcinoma, |
| | lymphoma/leukemia, mesenchymal tumors, secondary |
| | tumors (renal cell carcinoma, prostatic adenocarcinoma, |
| | melanoma, other metastasis from lung and breast) |

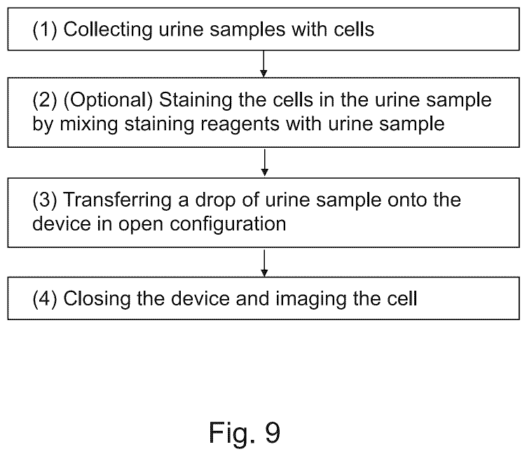

FIG. 9 illustrates a flow chart of one exemplary embodiment of the method with the steps of (1) collecting urine samples with cells inside a container; (2) staining the cells in the urine sample by mixing staining reagents with urine sample, (3) transferring a drop of urine sample onto the device in open configuration; (4) closing the device and imaging the cell sandwiched by two plates (plate 1 and plate 2).

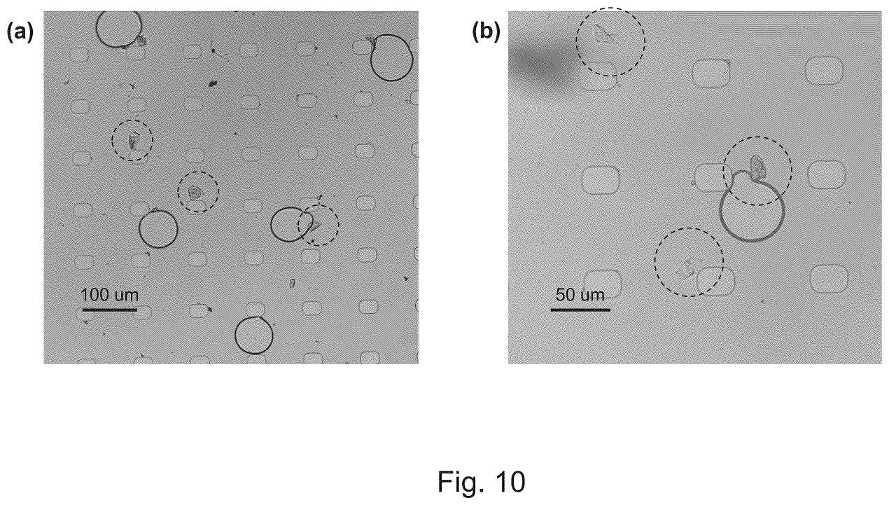

FIG. 10 illustrates example bright field microscopy images of Haematoxylin stained urine cells inside the device, wherein the device has a gap of 10 um, a pillar array of 30 by 40 um pillar size and 80 um inter spacing distance. The labeled cell inside the images are mainly stained epithelial cells.

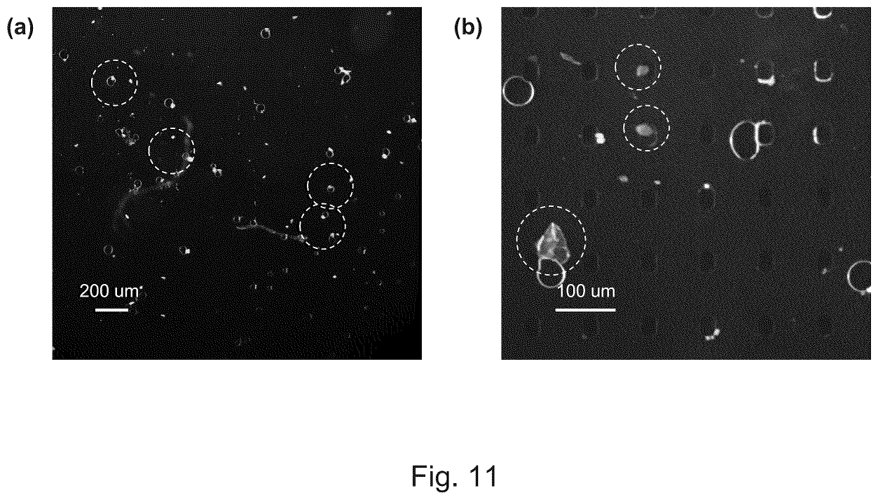

FIG. 11 illustrates example fluorescence images taken by smartphone of Acridine orange stained urine cells inside the device, wherein the device has a gap of 30 um, a pillar array of 30 by 40 um pillar size and 80 um inter spacing distance. The labeled cell inside the images are mainly stained epithelial cells and white blood cells.

We claim:

1. A method of rapidly detecting an analyte inside a sample by sandwiching the sample between two plates, binding the analyte inside a cell with a probe, and imaging, wherein the distance spacing between the two plates and the probe concentration are optimized for detection of the analyte in the cell without washing, comprising:
(a) obtaining a sample that contains at least a cell, wherein the sample contains or is suspected of containing an analyte, wherein the concentration of the analyte inside the cell is higher than that outside the cell;
(b) obtaining a probe that binds the analyte, wherein the probe has a configured probe concentration;
(c) obtaining a sample holder comprising a first plate and a second plate that are arranged or can be arranged to face each other with a configured plate-spacing between them;
(d) sandwiching the sample and the probe between the two plates to form a sample-probe thin layer, wherein the sample-probe thin layer comprises a volume A and a volume B, wherein the thickness of the volume A and the volume B are regulated by the plate-spacing between two plates, and wherein volume A contains at least a fraction of the cell, and volume B does not contain the cell;
(e) imaging, after step (d) and without washing away the unbound probe and the two plates are arranged at the configured spacing, the sample-probe thin layer to produce one or more images, wherein the one or more images comprises the probe in volumes A and B; and
(f) comparing the signal of the probe inside the cell in the image of volume A to the signal of the probe in the image of volume B to determine whether the sample contains the analyte;
wherein the configured probe concentration and the configured plate-spacing are configured to make, in imaging step (e), the signal of the probe inside the cell in the image of volume A distinguishable from the signal of the probe in the image of volume B, and
wherein the plate-spacing in imaged in step (e) is 250 um or less.

2. The method of claim 1 further comprises: (i) obtaining, after step (b), a permeabilization agent, and (ii) sandwiching, in the step (d), the permeabilization agent between the two plates and together with the sample and the probe.

3. The method of determining a disease and/or disorder of a subject, comprising:
(i) using the method of claim 1, wherein the sample is from the subject and the analyte is a biomarker of the disease and/or disorder; and
(ii) determining a disease and/or disorder of a subject using the results of step (f).

4. The method of any prior claim, wherein the imaging step (e) is performed after step (d), without separating the two plates.

5. The method of claim 3, wherein the disease and/or disorder is a viral infection, a bacterial infection, cancer or an inflammatory disease.

6. The method of claim 1, wherein the configured plate-spacing is regulated by spacers between the plates.

7. The method of any prior claim, where the spacing between the two plates is configured to make the saturation time for the binding between the analyte and the probe is less than 300 seconds.

8. The method of any prior claim, wherein each of volumes A and B has, during the imaging step (e), one surface in contact with the one of the two plates and another surface of in contact with other plate.

9. The method of any prior claim, wherein the probe comprises a binding agent that binds specifically to the analyte.

10. The method of any prior claim, wherein, before the imaging step (e), the method comprises permeabilizing the cell.

11. The method of any prior claim, wherein one or more of the plates comprises a dry permeabilizing agent and, in step (d), the permeabilizing agent dissolves in the sample and permeabilizes the cell.

12. The method of any prior claim, wherein the probe is comprised by a staining solution, wherein the staining solution and the sample are sandwiched between the two plates in step (e).

13. The method of any prior claim, wherein in the sample region being imaged in the step (e), the spacing between the two plates is configured to make the saturation time for the binding between the analyte and the probe 60 seconds or less.

14. The method of any prior claim, wherein the analyte is a protein, nucleic acid, or other macromolecule.

15. The method of any prior claim, wherein the probe is a fluorescent or luminescent probe.

16. The method of any prior claim, wherein the probe is a protein, nucleic acid, or aptamer.

17. The method of any prior claim, wherein the cell is a free cell or a tissue section.

18. The method of any prior claim, wherein the imaging of step (e) is done using a mobile phone.

19. The method of any prior claim, wherein the results of step (f) are transmitted to a remote location for analysis.

20. The method of any prior claim, wherein the cell is a human cell.

21. The method of any prior claim, wherein the cell is permeabilized either before or after the sample and the probe are sandwiched between the two plates.

22. The method of any prior claim, wherein the configured plate-spacing is configured to make the cells in the sample forming a monolayer of cells between the two plates.

23. The method of any prior claim, wherein one or both of the plates has a pillar that is used a reference for imaging the signal from the probe.

24. The method of any prior claim, wherein the method is multiplexed and comprises binding of multiple probes to the cell in step (d) and wherein the images of step (e) comprise signals from the multiple probes.

25. The method of any prior claim, wherein the sample is whole blood, without any liquid dilution.

26. The method of any prior claim, wherein the sample is whole blood, without any liquid dilution.

27. The method of any prior claim, wherein the probe is probe is coated on one or both plates before the sample is sandwiched between the two plates.

28. The method of any prior claim, wherein the configured plate-spacing have a 0.5 um, 1 um, 2 um, 3 um, 5 um, 10 um, 15 um, 20 um, 30 um, 50 um, or a range between any two of the values.

29. The method of claim 6, wherein the configured plate-spacing is 0.5 um, 1 um, 2 um, 3 um, 5 um, 10 um, 15 um, or a range between any two of the values.

30. The method of any prior claim, wherein the configured plate-spacing is 10 um.

31. The method of claim 1, wherein the imaging in step (e) is performed within 300 seconds after the step (d) of sandwiching the sample.

32. The method of any prior claim, wherein the first and second plates are movable relative to each other into different configurations, including an open configuration and a closed configuration, wherein the plate or the plates have spacers of uniform height on its surface, wherein in an open configuration, the two plates are completely or partially separated apart, the spacing between the plates is not regulated by the spacers, and wherein in a closed configuration, the thickness of the thin layer is regulated by the plates and the spacers.

33. The method of claim 6, wherein the spacers have a height of 0.5 um, 1 um, 2 um, 3 um, 5 um, 10 um, 15 um, 20 um, 30 um, 50 um, or a range between any two of the values.

34. The method of any prior claim further comprising determining the concentration of analyte outside the cell in the sample by measuring the signal of the analyte inside the cells.

35. The method of any prior claim further comprising determining the concentration of analyte outside the cell in the sample by measuring the signal of the analyte inside the cells and by obtaining a relationship between the concentration of the cell-free analyte in a sample and the signal of the analyte inside a cell.

36. The method of any prior claim, wherein the analyte is glycol-protein of virus.

37. The method of any prior claim, wherein the analyte is RNA of virus.

38. The method of any prior claim, wherein the analyte is RNA of COVID-19.

39. The method of any prior claim, wherein the sample comprises bodily fluid selected from the group consisting of amniotic fluid, aqueous humour, vitreous humour, blood, breast milk, cerebrospinal fluid (CSF), cerumen (earwax), chyle, chime, endolymph, perilymph, feces, breath, gastric acid, gastric juice, lymph, mucus, pericardial fluid, peritoneal fluid, pleural fluid, pus, rheum, saliva, exhaled breath condensates, sebum, semen, sputum, sweat, synovial fluid, tears, vomit, urine, and any combination thereof.
